SEQUENCE LISTING (436 sequences)

SEQ ID NO: 1 - protein, 99 aa, Artificial Sequence
  anti-HCV immunogenic peptide
  MISC_FEATURE (1)..(1): G, D, E, V or S
  MISC_FEATURE (5)..(5): A or V
  MISC_FEATURE (6)..(6): R, H or Q
  MISC_FEATURE (7)..(7): L, R, P, S or G
  MISC_FEATURE (8)..(8): G or E
  MISC_FEATURE (14)..(14): R, L or H
  MISC_FEATURE (16)..(16): L or P
  MISC_FEATURE (17)..(17): V, E or A
  MISC_FEATURE (18)..(18): E, V, D or G
  MISC_FEATURE (19)..(19): G or D
  MISC_FEATURE (20)..(20): D or V
  MISC_FEATURE (21)..(21): N or S
  MISC_FEATURE (23)..(23): S or F
  MISC_FEATURE (24)..(24): P or Q
  MISC_FEATURE (26)..(26): L, H, R, F, P or C
  MISC_FEATURE (27)..(27): A, V or I
  MISC_FEATURE (28)..(28): G, S, D, N, I or V
  MISC_FEATURE (31)..(31): A, V or E
  MISC_FEATURE (33)..(33): P, S or T
  MISC_FEATURE (35)..(35): L, P, H or R
  MISC_FEATURE (37)..(37): P or L
  MISC_FEATURE (39)..(39): T or I
  MISC_FEATURE (40)..(40): L, P or H
  MISC_FEATURE (42)..(42): P or L
  MISC_FEATURE (44)..(44): M or T
  MISC_FEATURE (45)..(45): A, V or P
  MISC_FEATURE (46)..(46): M, I or T
  MISC_FEATURE (48)..(48): A or V
  MISC_FEATURE (49)..(49): W, A, L or V
  MISC_FEATURE (50)..(50): G or D
  MISC_FEATURE (52)..(52): Q, L or R
  MISC_FEATURE (57)..(57): H, L, P or R
  MISC_FEATURE (59)..(59): V, A, E, K or T
  MISC_FEATURE (60)..(60): A or V
  MISC_FEATURE (61)..(61): L, R, H or P
  MISC_FEATURE (64)..(64): V, A, I or G
  MISC_FEATURE (67)..(67): P or L
  MISC_FEATURE (68)..(68): R, Q, L, M, T, E or P
  MISC_FEATURE (71)..(71): G or D
  MISC_FEATURE (72)..(72): V, A or G
  MISC_FEATURE (74)..(74): R or H
  MISC_FEATURE (75)..(75): V or A
  MISC_FEATURE (76)..(76): I or T
  MISC_FEATURE (79)..(79): R, G or K
  MISC_FEATURE (82)..(82): I or T
  MISC_FEATURE (83)..(83): P or L
  MISC_FEATURE (84)..(84): S or L
  MISC_FEATURE (85)..(85): H or R
  MISC_FEATURE (86)..(86): A or V
  MISC_FEATURE (87)..(87): A, V or G
  MISC_FEATURE (88)..(88): S or L
  MISC_FEATURE (90)..(90): T or I
  MISC_FEATURE (94)..(94): T or I
  MISC_FEATURE (95)..(95): F, Y or S
  MISC_FEATURE (97)..(97): S or L
  MISC_FEATURE (99)..(99): A, V, G or H
XWVCXXXXRLPSGXNXXXXXXLXXRXXXPRXGXGXSXGXXGXSXXXRXXXGXDGSCXPXXXGLXGAXXTPXXGXXXWVXSSXXXXXXXPXSWGXXRXSX

SEQ ID NO: 2 - protein, 99 aa, Artificial Sequence
  anti-HCV immunogenic peptide
GWVCARLGRLPSGRNLVEGDNLSPRLAGPRAGPGLSPGTLGPSMAMRAWGGQDGSCHPVALGLVGAPRTPGVGRVIWVRSSIPSHAASPTSWGTFRSSA

SEQ ID NO: 3 - protein, 99 aa, Artificial Sequence
  anti-HCV immunogenic peptide
GWVCARLGRLPSGRNLVEGDNLSPRLAGPRVGPGLSPGTLGPSMAMRAWGGQDGSCHPAVLGLVGAPQTPGVGRVIWVKSSIPSHAASPTSWGTSRSSA

SEQ ID NO: 4 - protein, 99 aa, Artificial Sequence
  anti-HCV immunogenic peptide
GWVCARLGRLPSGRNLVEGDNLSPRLAGPRVGPGLSPGTLGPSMAMRAWGGQDGSCHPAALGLVGAPQTPGVGRVIWVRSSIPSHAASPTSWGTSRSSA

SEQ ID NO: 5 - protein, 99 aa, Artificial Sequence
  anti-HCV immunogenic peptide
GWVCARLGRLPSGRNLVEGDNLSPRLAGPRVGPGLSPGTLGPSLAMRAWGGQDGSCHPAALGLVGAPQTPGVGRVTWVKSSIPSHAASPTSWGTFRSSA

SEQ ID NO: 6 - protein, 99 aa, Artificial Sequence
  anti-HCV immunogenic peptide
GWVCVRLGRLPSGRNLVEGDNLSPRLAGPRAGPGLSLGILGPSMAMRAWGGQDGSCHPAALGLVGALMTPGVGRVIWVRSSIPLHAASPTSWGTSRLSA

SEQ ID NO: 7 - protein, 99 aa, Artificial Sequence
  anti-HCV immunogenic peptide
GWVCVRLGRLPSGRNLVEGDNLSPRLAGPRAGPGLSLGILGPSMAMRAWGGQDGSCHPAALGLVGAPMTPGVGRVIWVRSSIPLHAASPTSWGTFRSSA

SEQ ID NO: 8 - protein, 99 aa, Artificial Sequence
  anti-HCV immunogenic peptide
GWVCARLGRLPSGRNLVEGDNLSPRLAGPRAGPGLSPGILGPSMAMRAWGGQDGSCHPAALGLAGALPTPGVGRVTWVRSSIPLHAASPTSWGTFRSSA

SEQ ID NO: 9 - protein, 99 aa, Artificial Sequence
  anti-HCV immunogenic peptide
GWVCARLGRLPSGRNLVEGDNLSPRLAGPRAGPGLSLGTLGPSMAMRAWGGQDGSCHPAALGLVGAPQTPGVGRVIWVRSSIPLHAASPTSWGTFRSSA

SEQ ID NO: 10 - protein, 99 aa, Artificial Sequence
  anti-HCV immunogenic peptide
GWVCARLGRLPSGRNLVEGDNLSPRLAGPRAGPGLSPGTLGPSMAMRAWGGQDGSCHPAALGLVGAPLTPGVGRVIWVRSSIPSHAASPTSWGTFRSSA

SEQ ID NO: 11 - protein, 99 aa, Artificial Sequence
  anti-HCV immunogenic peptide
GWVCAHLGRLPSGRNLVEGDNLFPRLVGPRAGPGLSPGTLGPSMAMRAWGGQDGSCHPEALGLVGAPLTPGVGRVIWVRSSIPSHAASPTSWGTFRSSA

SEQ ID NO: 12 - protein, 99 aa, Artificial Sequence
  anti-HCV immunogenic peptide
GWVCARLGRLPSGRNLVEGDNLSPRLAGPRAGPGLSPGTLGPSMAMRAWGGQDGSCHPAALGLVGAPTTPGVGRVIWVRSSIPSHAASPTSWGTFRSSA

SEQ ID NO: 13 - protein, 99 aa, Artificial Sequence
  anti-HCV immunogenic peptide
GWVCARLGRLPSGRNLVEGDNLSPRLAGPRAGPGLSPGTLGPSMAMRAWGGQDGSCHPAALGLVGAPMTPGVGRVIWVRSSIPSHAASPTSWGTFRSSA

SEQ ID NO: 14 - protein, 99 aa, Artificial Sequence
  anti-HCV immunogenic peptide
GWVCARLGRLPSGRNLAEGDNLSPRLAGPRAGPGLSPGTLGPSMAMRAWGGQDGSCHPAALGLVGAPRTPGVGRAIWVRSSIPSHAASPTSWGTFRSSA

SEQ ID NO: 15 - protein, 99 aa, Artificial Sequence
  anti-HCV immunogenic peptide
GWVCARLGRLPSGRNLVEGDNLSPRLANPRAGPGLSPGTLGPSMAMRAWGGQDGSCHPAALGLVGAPRTPGVGRAIWVRSSIPSHAASPTSWGTFRSSA

SEQ ID NO: 16 - protein, 99 aa, Artificial Sequence
  anti-HCV immunogenic peptide
GWVCARLGRLPSGHNLAEGDNLSPRLAIPRAGPGLSLGTLGPSMAMRAWGGQDGSCHPAALGLVGAPRTPGVGRVIWVRSSIPSHAALPTSWGTFRSSA

SEQ ID NO: 17 - protein, 99 aa, Artificial Sequence
  anti-HCV immunogenic peptide
GWVCARLGRLPSGHNLAEGDNLSPRLAIPRAGPGLSLGTLGPSMAMRAWGGQDGSCRPAALGLVGAPRTPGVGRVIWVRSSIPSHAALPTSWGTFRSSA

SEQ ID NO: 18 - protein, 99 aa, Artificial Sequence
  anti-HCV immunogenic peptide
VWVCARLGRLPSGHNLVEGDNLFPRLAGPRAGPGLSLGTLGPSMAMRAWGGQDGSCHPAALGLVGAPLTPGVGRAIWVRSSIPSHAASPISWGTFRSSA

SEQ ID NO: 19 - protein, 99 aa, Artificial Sequence
  anti-HCV immunogenic peptide
GWVCARLGRLPSGHNLVEGDNLSPRLAGPRAGPGLSPGTLGPSTAMRAWGGQDGSCHPVALGLVGAPRTPGVGRVIWVRSSIPSHAASPTSWGTFRSSA

SEQ ID NO: 20 - protein, 99 aa, Artificial Sequence
  anti-HCV immunogenic peptide
GWVCARLGRLPSGRNLVEGDNLSPRFAGPRAGPGLSPGTLGPSTAMRAWGGQDGSCHPAALGLVGAPRTPGVGRVTWVRSSIPSHAASPISWGTFRSSA

SEQ ID NO: 21 - protein, 99 aa, Artificial Sequence
  anti-HCV immunogenic peptide
GWVCARLGRLPSGRNLVEGDNLSPRLAGPRAGPGLSPGTLGPSTAMRAWGGQDGSCHPAALGLVGAPRTPGVGRVIWVRSSIPSHAASPTSWGTFRSSA

SEQ ID NO: 22 - protein, 99 aa, Artificial Sequence
  anti-HCV immunogenic peptide
GWVCARLGRLPSGRNLVEGDNLSPRFADPRAGPGLSPGTLGPSMPTRVWGGQDGSCHPVALGLVGAPRTPGVGRAIWVRSSIPSRAASPTSWGTFRSSA

SEQ ID NO: 23 - protein, 99 aa, Artificial Sequence
  anti-HCV immunogenic peptide
GWVCARLGRLPSGRNLVEGDNLSPRLADPRAGPGLSPGTLGPSMPTRVWGGQDGSCHPEAPGLVGAPRTPGVGRAIWVRSSIPLHAASPTSWGTFRSSA

SEQ ID NO: 24 - protein, 99 aa, Artificial Sequence
  anti-HCV immunogenic peptide
GWVCARLGRLPSGRNLVEGDNLSPRLAGPRAGPGLSPGTLGPSMVTRVWGGQDGSCHPEALGLAGAPRTPGVGRVIWVRSSIPLRAASPTSWGTFRSSA

SEQ ID NO: 25 - protein, 99 aa, Artificial sequence
  anti-HCV immunogenic peptide
GWVCARLGRLPSGRNLVEGDNLSPRLADPRAGPGLSPGTLGPSMAMRVWGGQDGSCHPEALGLVGAPRTPGVGRAIWVRSSIPLHAASPTSWGTFRSSA

SEQ ID NO: 26 - protein, 99 aa, Artificial Sequence
  anti-HCV immunogenic peptide
GWVCARLGRLPSGRNLVEGDNLSPRLAGPRAGPGLSPGTLGPSMAMRVWGGQDGSCHPGALGLVGAPRTPGVGRVIWVRSSIPLHAASPTSWGTFRLSA

SEQ ID NO: 27 - protein, 99 aa, Artificial Sequence
  anti-HCV immunogenic peptide
GWVCARLGRLPSGRNLVEGDNLSPRLAGPRAGPGLSPGTLGPSTAMRVWGGQDGSCHPEALGLVGAPRTPGVGRVIWVRSSIPLHAASPTSWGTFRLSA

SEQ ID NO: 28 - protein, 99 aa, Artificial Sequence
  anti-HCV immunogenic peptide
SWVCARLGRLPSGRNLVEGDNLSPRLAGPRAGPGPSPGTPGPSMAMRVWGGQDGSCHPEALGLVGAPRTPGVGRVIWVRSSIPLHAASPTSWGTFRSSA

SEQ ID NO: 29 - protein, 99 aa, Artificial Sequence
  anti-HCV immunogenic peptide
GWVCARLGRLPSGRNLVEGDNLSPRFAGPRAGPGLSPGTLGPSMATRVWGGQDGSCHPVALGLIGAPRTPGVGRVIWVRSSIPLHAGSPTSWGTFRSSA

SEQ ID NO: 30 - protein, 99 aa, Artificial Sequence
  anti-HCV immunogenic peptide
GWVCARLGRLPSGRNLVEGDNLSPRFAGPRAGPGLSPGTLGPSMAMRVWGGQDGSCHPGALGLVGAPRTPGVGRVIWVRSSIPLHAGSPTSWGTFRSSA

SEQ ID NO: 31 - protein, 99 aa, Artificial Sequence
  anti-HCV immunogenic peptide
GWVCARLGRLPSGRNLVEGDNLSPRLAGPRVGPGLSPGTLGPSMATRVWGGQDGSCHPVALGLVGAPQTPGVGRVIWVRSSIPLHAASPTSWGTFRLSA

SEQ ID NO: 32 - protein, 99 aa, Artificial Sequence
  anti-HCV immunogenic peptide
GWVCARLGRLPSGRNIVEGDNLSPRLAGPRVGPGLSPGTLGPSMATRVWGGQDGSCHPVALGLVGAPQTPGVGRVIWVRSSIPLHAASPTSWGTFRLSA

SEQ ID NO: 33 - protein, 99 aa, Artificial Sequence
  anti-HCV immunogenic peptide
GWVCARLGRLPSGRNLVEGHNLSPRLAGPRVGPGLSPGTLGPSMATRVWGGQDGSCHPVARGLVGAPRTPGVGRAIWVRSSIPLHAASPTSWGTFRLSA

SEQ ID NO: 34 - protein, 99 aa, Artificial Sequence
  anti-HCV immunogenic peptide
GWVCARGGRLPSGRNLVEADNLSPRLAVPREGPGLSPGTLGPSMATRVWGGLDGSCHPEAPGLVGAPRTPGVGRVIWVRSSIPSHAASPTSWGTFRSSA

SEQ ID NO: 35 - protein, 99 aa, Artificial Sequence
  anti-HCV immunogenic peptide
GWVCARLGRLPSGRNLVEGDNLSPRLAGPRAGPGLSPGTLGPSMATRVWGGQDGSCHPEAPGLVGAPRTPGVGRVIWVRSSIPSHAASPTSWGTFRSSA

SEQ ID NO: 36 - protein, 99 aa, Artificial Sequence
  anti-HCV immunogenic peptide
GWVCARLGRLPSGRNLVEGDNLSPRLAGPRAGPGLSPGTLGPSMAMRVWGGQDGSCHPEALGLVGAPRTPGVGRVIWVRSSIPSQVASPTSWGTFRSSA

SEQ ID NO: 37 - protein, 99 aa, Artificial Sequence
  anti-HCV immunogenic peptide
GWVCARLGRLPSGRNLVEGDNLSPRLAGPRAGPGLSPGTPGPSMAMRVWGGQDGSCHPAALGLVGAPQTPGAGRVIWVRSSIPSHAASPTSWGTFRSSA

SEQ ID NO: 38 - protein, 99 aa, Artificial Sequence
  anti-HCV immunogenic peptide
GWVCARLGRLPSGRNLVEGDNLSPRLAGPRAGPGLSPGTPGPSMAMRVWGGQDGSCHPAALGLVGAPQTPGAGRVIWVGSSIPSHAASPTSWGTFRSSA

SEQ ID NO: 39 - protein, 99 aa, Artificial Sequence
  anti-HCV immunogenic peptide
GWVCARLGRLPSGRNLVEGDNLSPRLAGPRAGPGLSPGTPGPSMAMRVWGGQDGSCHPVALGLVGAPRTPGVGRVIWVRSSIPSHAASPTSWGTFRSSA

SEQ ID NO: 40 - protein, 99 aa, Artificial Sequence
  anti-HCV immunogenic peptide
GWVCARLGRLPSGRNLVEGDNLSPRLAGPRAGPGLSPGTLGLSMAMRAWGGQDGSCHPVALGLVGAPRTPGVGRVTWVRSSIPLHAASPTSWGTFRSSA

SEQ ID NO: 41 - protein, 99 aa, Artificial Sequence
  anti-HCV immunogenic peptide
GWVCARLGRLPSGRNLVEGDNLSPRLAGPRAGPGLSPGTLGLSMAMRVWGGQDGSCRPVALGLVGAPRTPGVGRVIWVRSSIPSHAASPTSWGTFRSSA

SEQ ID NO: 42 - protein, 99 aa, Artificial Sequence
  anti-HCV immunogenic peptide
GWVCARLGRLPSGRNLVEGDNLSPRLAGPRAGPGLSPGTLGLSMAMRVWGGQDGSCRPVAPGLVGAPRTPGVGRVIWVRSSIPSHAASPTSWGTFRSSA

SEQ ID NO: 43 - protein, 99 aa, Artificial Sequence
  anti-HCV immunogenic peptide
GWVCARLGRLPSGRNLVEGDNLSPRLAGPRAGPGLSPGTLGPSMAMRAWGGQDGSCHPVALGLVGAPQTPGVGRVIWVRSSIPSHAASPTSWGTFRLSA

SEQ ID NO: 44 - protein, 99 aa, Artificial Sequence
  anti-HCV immunogenic peptide
GWVCARLGRLPSGRNLVEGDNLSPRLAGPRAGPGLSPGTLGPSMAMRAWGGQDGSCHPVALGLVGAPQTPGVGRVIWVRSSIPSHAASPTSWGTFRSSA

SEQ ID NO: 45 - protein, 99 aa, Artificial Sequence
  anti-HCV immunogenic peptide
GWVCARLGRLPSGRNLVEGDNLSPRLADPRAGPGLSPGILGPSMAMRALGGQDGSCHPAAPGLVGAPRTPGVGRVIWVRSSIPSHAASPTSWGTFRSSA

SEQ ID NO: 46 - protein, 99 aa, Artificial Sequence
  anti-HCV immunogenic peptide
GWVCARLGRLPSGRNLVEGDNLSPRLADPRAGPGLSPGTLGPSMAMRAWGGQDGSCHPAAPGLVGAPRTPGVGRVIWVRSSIPSHAASPTSWGIFRSSA

SEQ ID NO: 47 - protein, 99 aa, Artificial Sequence
  anti-HCV immunogenic peptide
GWVCARLGRLPSGRNLVDGDNLSPRLADPRAGPGLSPGTLGPSMAMRAWGGQDGSCHPAAPGLVGAPRTPGVGRVTWVRSSIPLHAASPISWGIFRSSA

SEQ ID NO: 48 - protein, 99 aa, Artificial Sequence
  anti-HCV immunogenic peptide
GWVCARLGRLPSGRNLVEGDNLSQRLANPRAGPGLSPGTLGPSMAMRAWGGQDGSCHPAAPGLVGAPRTPGVGRVTWVRSSIPLHAASPISWGTFRSSA

SEQ ID NO: 49 - protein, 99 aa, Artificial Sequence
  anti-HCV immunogenic peptide
GWVCVRLGRLPSGRNLVEGDNLSQRLANPRAGPGLSPGTLGPSMAMRAWGGQDGSCHPAAPGLVGAPRTPGVGRVTWVRSSIPLHAASPISWGTFRSSA

SEQ ID NO: 50 - protein, 99 aa, Artificial Sequence
  anti-HCV immunogenic peptide
GWVCARLGRLPSGRNLVEGDNLSPRLANPRAGPGLSPGTLGPSMAMRAWGGQDGSCHPVAPGLVGAPRTPGVGRVIWVRSSIPLHAASPISWGTFRSSA

SEQ ID NO: 51 - protein, 99 aa, Artificial Sequence
  anti-HCV immunogenic peptide
GWVCARLGRLPSGRNLVEGDNLSPRLVDPRAGPGLSPGTLGPSMAMRAWGGQDGSCHPAAPGLVGAPRTPGVGRVIWVRSSIPLHAASPTSWGTFRSSA

SEQ ID NO: 52 - protein, 99 aa, Artificial Sequence
  anti-HCV immunogenic peptide
GWVCARLGRLPSGRNLVEGDNLSPRLANPRVGPGLSPGTLGPSMAMRAWGGQDGSCHPAAPGLVGAPRTPGVGRVIWVRSSIPSHAASPISWGTFRSSA

SEQ ID NO: 53 - protein, 99 aa, Artificial Sequence
  anti-HCV immunogenic peptide
GWVCARLGRLPSGRNLVEGDNLSPRLANPRVGPGLSPGTLGPSMAMRAWGGQDGSCHPAAPGLVGAPRTPGVGRVIWVRSSIPSHAASPTSWGTFRSSA

SEQ ID NO: 54 - protein, 99 aa, Artificial Sequence
  anti-HCV immunogenic peptide
GWVCARPGRLPSGRNLVEGDNLSPRLAGPRAGPGLSPGTLGLSMAMRAWGGQDGSCHPAAPGLVGAPRTPGVGRVIWVRSSIPSHAASPISWGTFRSSA

SEQ ID NO: 55 - protein, 99 aa, Artificial Sequence
  anti-HCV immunogenic peptide
GWVCARLGRLPSGRNLVEGDNLSPRLAGPRAGPGLSPGTLGPSMAMRAWGGQDGSCHPAAPGLVGAPRTPGVGRVTWVRSSIPSHAASPTSWGTFRSSA

SEQ ID NO: 56 - protein, 99 aa, Artificial Sequence
  anti-HCV immunogenic peptide
GWVCARLGRLPSGRNLVEGDSLSPRLAGPRVGPGLSPGTLGPSMAMRVWGGQDGSCHPVAPGLVGAPPTPGVGRVIWVRSSIPSHAASPTSWGTFRSSA

SEQ ID NO: 57 - protein, 99 aa, Artificial Sequence
  anti-HCV immunogenic peptide
GWVCARLGRLPSGRNLVEGDSLSPRLAGPRAGPGLSPGTLGPSMAMRVWGGQDGSCHPKALGLVGAPETPGVGRVTWVRSSIPSHAASPTSWGTFRSSA

SEQ ID NO: 58 - protein, 99 aa, Artificial Sequence
  anti-HCV immunogenic peptide
GWVCARLGRLPSGRNLVEGDSLSPRLASPRAGPGLSPGTLGPSMAMRAWGGQDGSCHPVAPGLVGAPQTPGVGRVIWVRSSIPSHAASPTSWGTFRSSA

SEQ ID NO: 59 - protein, 99 aa, Artificial Sequence
  anti-HCV immunogenic peptide
GWVCARLGRLPSGRNLVEGDSLSPRLAGPRAGPGHSPGTLGPSMAMRAWGGQDGSCHPVAPGLVGAPRTPGVGRVIWVRSSIPSHAASPTSWGTFRSSA

SEQ ID NO: 60 - protein, 99 aa, Artificial Sequence
  anti-HCV immunogenic peptide
GWVCARLGRLPSGRNLVEGDSLSPRLAGPRAGPGLSPGTLGPSMAMRAWGGQDGSCHPAAPGLVGAPRTPGVGRVIWVRSSTPSHAASPTSWGTFRSSA

SEQ ID NO: 61 - protein, 99 aa, Artificial Sequence
  anti-HCV immunogenic peptide
GWVCARSGRLPSGRNPVEGDSLSPRLAGPRAGPGLSPGILGPSMAMRAWGGQDGSCHPAAPGLVGAPRTPGVGRVIWVRSSIPSHAASPTSWGTFRSSA

SEQ ID NO: 62 - protein, 99 aa, Artificial Sequence
  anti-HCV immunogenic peptide
GWVCARLGRLPSGRNLVEGDSLSPRLAGPRAGPGLSPGTLGPSMAMRAWGGQDGSCHPAALGLVGAPRTPGVGRVIWVRSSIPSHAASPTSWGTFRSSA

SEQ ID NO: 63 - protein, 99 aa, Artificial Sequence
  anti-HCV immunogenic peptide
GWVCARLGRLPSGRNLVEGDNLSPRLAGPRAGPGLSPGILGPSMAMRAWGGQDGSCRPVAPGLVGAPRTPGVGRVIWVRSSIPSHAASPTSWGTFRSSA

SEQ ID NO: 64 - protein, 99 aa, Artificial Sequence
  anti-HCV immunogenic peptide
GWVCARLGRLPSGRNLVEGDNLSPRLAGPRAGPGLSPGILGPSTATRAWGGQDGSCHPVVPGLVGAPRTPGVGRVIWVRSSIPSHAASPTSWGTFRSSA

SEQ ID NO: 65 - protein, 99 aa, Artificial Sequence
  anti-HCV immunogenic peptide
GWVCARLGRLPSGRNLVEGDNLSPRLAGPRAGPGRSPGTLGPSMAMRAWGGQDGSCHPVAPGLVGAPRTPGVGRVIWVRSSIPSHAASPTSWGIFRSSA

SEQ ID NO: 66 - protein, 99 aa, Artificial Sequence
  anti-HCV immunogenic peptide
GWVCARLGRLPSGRNLVEGDSLSPRLANPRAGPGLSPGTLGPSMATRAWGGQDGSCLPVAPGLVGAPQTPGVGRVIWVRSSTPSHAASPTSWGTFRLSA

SEQ ID NO: 67 - protein, 99 aa, Artificial Sequence
  anti-HCV immunogenic peptide
GWVCARLGRLPSGRNLVEGDSLSPRLANPRAGPGLSPGTLGPSMATRVWGGQDGSCLPVAPGLVGAPQTPGVGRVIWVRSSTPSHAASPTSWGTFRSSA

SEQ ID NO: 68 - protein, 99 aa, Artificial Sequence
  anti-HCV immunogenic peptide
GWVCARLGRLPSGRNLVEGDNLSPRLAGPRAGPGLSPGTLGPSMATRVWGGQDGSCHPVAPGLVGAPQTPGVGRVIWVRSSIPSHAASPTSWGTSRSSA

SEQ ID NO: 69 - protein, 99 aa, Artificial Sequence
  anti-HCV immunogenic peptide
GWVCARLGRLPSGRNLVEGDNLSPRFASPRAGPGLSPGTLGPSMATRAWGGQDGSCHPVAPGLVGAPQTPGVGRVIWVRSSIPSHAASPTSWGTFRSSA

SEQ ID NO: 70 - protein, 99 aa, Artificial Sequence
  anti-HCV immunogenic peptide
GWVCARLGRLPSGRNLVEGDNLSPRLAGPRVGPGLSPGTLGPSMATRAWGGQDGSCHPAAPGLVGAPQTPGVGRVIWVRSSIPSHAASPTSWGTFRSSA

SEQ ID NO: 71 - protein, 99 aa, Artificial Sequence
  anti-HCV immunogenic peptide
GWVCARLGRLPSGRNLVEGDNLSPRLAGPRVGPGLSPGTLGPSMATRAWGGQDGSCHPAAPGLIGAPQTPGVGRVIWVRSSIPSHAASPTSWGTFRSSA

SEQ ID NO: 72 - protein, 99 aa, Artificial Sequence
  anti-HCV immunogenic peptide
GWVCARLGRLPSGRNLVEGDNLSPRLAGPRVGPGLSPGTLGPSMATRAWGGQDGSCHPAAPGLVGAPPTPGVGRVIWVRSSIPSHAASPTSWGTSRSSA

SEQ ID NO: 73 - protein, 99 aa, Artificial Sequence
  anti-HCV immunogenic peptide
GWVCARLGRLPSGRNLVEGDNLSPRLAGPRVGPGLSPGTLGPSMATRAWGGQDGSCHPVAPGLVGAPQTPGVGRVIWVRSSIPSHAASPTSWGTFRSSA

SEQ ID NO: 74 - protein, 99 aa, Artificial Sequence
  anti-HCV immunogenic peptide
GWVCARLGRLPSGRNLVEGDNLSPRLAGPRAGPGLSPGTLGPSMATRAWGGQDGSCHPVAPGLVGAPRTPGVGRVIWVRSSIPSHAASPTSWGTFRSSA

SEQ ID NO: 75 - protein, 99 aa, Artificial Sequence
  anti-HCV immunogenic peptide
GWVCARLGRLPSGRNLVEGDNLSPRLAGPRAGPGLSPGTLGPSMATRAWGGQDGSCHPVAPGLVGAPVTPGVGRVIWVRSSIPSHAASPTSWGTFRSSA

SEQ ID NO: 76 - protein, 99 aa, Artificial Sequence
  anti-HCV immunogenic peptide
GWVCARLGRLPSGRNLVEGDNLSPRLAGPRAGPGLSPGTLGPSMATRAWGGQDGSCHPAAPGLVGAPTTPGVGRVIWVRSSIPSHAASPTSWGTFRLSA

SEQ ID NO: 77 - protein, 99 aa, Artificial Sequence
  anti-HCV immunogenic peptide
GWVCARLGRLPSGRNLVEGDNLSPRLAGPRAGPGLSPGTLGPSMATRAWGGQDGSCHPAAPGLVGAPTTPGVGRVIWVRSSIPLHAASPTSWGTFRLSA

SEQ ID NO: 78 - protein, 99 aa, Artificial Sequence
  anti-HCV immunogenic peptide
GWVCARLGRLPSGRNLVEGDNLSPRLASPRAGPGLSPGTLGPSMATRAWGGQDGSCHPAAPGLVGAPTTPGVGRVIWVRSSIPSHAASPTSWGTFRLSA

SEQ ID NO: 79 - protein, 99 aa, Artificial Sequence
  anti-HCV immunogenic peptide
GWVCARLGRLPSGRNLVEGDNLSPRLASPRAGPGLSPGTLGPSMATRAWGGQDGSCHPAAPGLVGAPMTPGVGRVIWVRSSIPSHAASPTSWGTFRLSA

SEQ ID NO: 80 - protein, 99 aa, Artificial Sequence
  anti-HCV immunogenic peptide
GWVCARLGRLPSGHNLVEGDNLSPRLAGPRAGPGLSPGTLGPSMATRAWGGQDGSCHPAAPGLVGAPRTPGVGRVIWVRSSIPSHAASPTSWGTFRLSA

SEQ ID NO: 81 - protein, 99 aa, Artificial Sequence
  anti-HCV immunogenic peptide
GWVCARLGRLPSGHNLVEGDNLSPRLAGPRVGPGLSPGTLGPSMATRAWGGQDGSCHPAAPGLVGAPRTPGVGRVIWVRSSIPSHAASPTSWGTFRSSA

SEQ ID NO: 82 - protein, 99 aa, Artificial Sequence
  anti-HCV immunogenic peptide
GWVCARLGRLPSGHNLVEGDNLSPRLASPRAGPGLSPGTLGPSMATRAWGGQDGSCHPAAPGLVGAPRTPGVGRVIWVRSSIPSHAASPTSWGTFRSSA

SEQ ID NO: 83 - protein, 99 aa, Artificial Sequence
  anti-HCV immunogenic peptide
GWVCARLGRLPSGRNLVEGDNLSPRLAGPRAGPGLSPGTLGPSMVTRAWGGQDGSCHPAAPGLVGAPRTPGVGRVIWVRSSIPSHAASPTSWGTFRSSA

SEQ ID NO: 84 - protein, 99 aa, Artificial Sequence
  anti-HCV immunogenic peptide
GWVCARLGRLPSGRNLVEGDNLSPRLAGPRAGPGLSPGTLGPSMATRAWGGQDGSCHPAAPGLVGAPRTPGVGRVIWVRSSIPSHAASPTSWGTFRSSA

SEQ ID NO: 85 - protein, 99 aa, Artificial Sequence
  anti-HCV immunogenic peptide
GWVCARLGRLPSGRNLVEGDNLSPRLAGPRAGPGLSPGTLGPSMATRAWGGQDGSCPPAAPGLVGAPRTPGVGRVIWVRSSIPSHAASPTSWGTFRSSA

SEQ ID NO: 86 - protein, 99 aa, Artificial Sequence
  anti-HCV immunogenic peptide
GWVCARLGRLPSGRNLVEGDNLSPRLASPRAGPGLSPGTLGPSMATRAWGGQDGSCHPVALGLVGAPRTPGVGRVIWVRSSIPSHAASPTSWGTFRLSA

SEQ ID NO: 87 - protein, 99 aa, Artificial sequence
  anti-HCV immunogenic peptide
GWVCARLGRLPSGRNLVEGDNLSPRLASPRAGPGLSPGTLGPSMATRAWGGQDGSCHPVAPGLVGAPRTPGVGRVIWVRSSIPSHAASPTSWGTFRSSA

SEQ ID NO: 88 - protein, 99 aa, Artificial Sequence
  anti-HCV immunogenic peptide
GWVCAQLGRLPSGRNLVEGDNLSPRLAGPRAGPGPSPGTLGPSMVTRAWDGQDGSCHPVALGLVGAPLTPGVGRVIWVRSSIPSHAASPTSWGTFRSSA

SEQ ID NO: 89 - protein, 99 aa, Artificial Sequence
  anti-HCV immunogenic peptide
GWVCARLGRLPSGRNLVEGDSLSPRLAGPRAGPGLSPGTLGPSMVTRAWGGQDGSCHPAALGLIGALQTPGVGRVIWVRSSIPSHAASPTSWGTFRSSA

SEQ ID NO: 90 - protein, 99 aa, Artificial Sequence
  anti-HCV immunogenic peptide
GWVCARLGRLPSGRNLVVGDNLSPRLADPRAGPGLSPGTLGPSMVIRAWGGQDGSCPPVALGLVGAPMTPGVGRVIWVRSSIPSHAVSPTSWGTFRSSA

SEQ ID NO: 91 - protein, 99 aa, Artificial Sequence
  anti-HCV immunogenic peptide
GWVCARLGRLPSGRNLVEGDNLSPRLASPRAGPGLSPGILGPSMAMRAWGGQDGSCHPVALGLVGAPRTPGVGRVIWVRSSIPSHAASPTSWGTFRSSA

SEQ ID NO: 92 - protein, 99 aa, Artificial Sequence
  anti-HCV immunogenic peptide
GWVCARLGRLPSGRNLVEGDNLSPRLASPRAGPGLSPGIPGPSMAMRAWGGQDGSCHPVALGLVGAPRTPGVGRVIWVRSSIPSHAASPTSWGTFRSSA

SEQ ID NO: 93 - protein, 99 aa, Artificial Sequence
  anti-HCV immunogenic peptide
GWVCARLGRLPSGRNLAEGDNLSPRLASPRAGPGLSPGTHGPSMAMRAWGGQDGSCHPVALGLVGAPRTPGVGRVIWVRSSIPSHAASPTSWGTFRSSA

SEQ ID NO: 94 - protein, 99 aa, Artificial Sequence
  anti-HCV immunogenic peptide
GWVCARLGRLPSGRNLVEGDNLSPRLASPRAGPGLSPGTLGPSMAMRAWGGQDGSCHPVALGLVGAPRTPGVGRVIWVRSSIPSHAASPTSWGTFRSSA

SEQ ID NO: 95 - protein, 99 aa, Artificial Sequence
  anti-HCV immunogenic peptide
GWVCARRGRLPSGRNLLEGDNLSPRLASPRAGPGLSPGTLGPSMATRAWGGQDGSCHPEALGLIGAPRTPGVGRVIWVKSSTPSHAASPTSWGTFRSSA

SEQ ID NO: 96 - protein, 99 aa, Artificial Sequence
  anti-HCV immunogenic peptide
GWVCARLGRLPSGRNLVEGDNLSPRLASPRAGPGLSPGTLGPSMAMRAWGGQDGSCHPAALGLIGAPRTPGVGRVIWVKSSTPSHAASPTSWGTSRSSA

SEQ ID NO: 97 - protein, 99 aa, Artificial Sequence
  anti-HCV immunogenic peptide
GWVCARLGRLPSGRNLVEGDNLSPRLASPRAGPGLSPGTLGPSMAMRAWGGQDGSCHPVALGLVGAPRTPGVGRVIWVKSSIPSHAASPTSWGTFRSSA

SEQ ID NO: 98 - protein, 99 aa, Artificial Sequence
  anti-HCV immunogenic peptide
GWVCARRGRLPSGRNLVEGDNLSPRLASPRAGPGPSPGTLGPSMAMRAWGGQDGSCHPVALGLVGAPRTPGVGRVIWVRSSIPSHAASPTSWGTFRSSA

SEQ ID NO: 99 - protein, 99 aa, Artificial Sequence
  anti-HCV immunogenic peptide
GWVCARLGRLPSGRNLVEGDNLSPRLASPRAGPGLSPGTLGPSMAMRAWGGQDGSCHPAALGLVGAPRTPGVGRVIWVRSSIPSHAGSPTSWGTFRSSA

SEQ ID NO: 100 - protein, 99 aa, Artificial Sequence
  anti-HCV immunogenic peptide
GWVCARLGRLPSGRNLVEGDNLSPRLASPRAGPGLSPGTLGPSMAMRAWGGQDGSCHPAALGLVGAPQTPGVGRVIWVRSSIPSHAGSPTSWGTFRSSG

SEQ ID NO: 101 - protein, 99 aa, Artificial Sequence
  anti-HCV immunogenic peptide
GWVCARLGRLPSGRNLVEGDNLSPRFASPRAGPGLSPGTLGPSMAMRAWGGQDGSCLPAALGLVGAPMTPGVGRVIWVRSSIPSHAASPTSWGTFRSSA

SEQ ID NO: 102 - protein, 99 aa, Artificial Sequence
  anti-HCV immunogenic peptide
GWVCARLGRLPSGRNLVEGDNLSPRLASPRAGPGLSPGTLGPSMAMRAWGGQDGSCLPAAHGLVGAPMTPGVGRVIWVRSSIPSHAASPTSWGTFRSSA

SEQ ID NO: 103 - protein, 99 aa, Artificial Sequence
  anti-HCV immunogenic peptide
GWVCARLGRLPSGRNLVEGDNLSPRLASPRAGPGLSPGTLGPSMAMRAVGGQDGSCHPAALGLVGAPRTPGVGRVIWVRSSIPSHAASPTSWGTFRSSA

SEQ ID NO: 104 - protein, 99 aa, Artificial Sequence
  anti-HCV immunogenic peptide
GWVCARLGRLPSGRNLVEGDNLSPRLASPRVGPGLSPGTLGPSMAMRAWGGQDGSCHPAALGLVGAPRTPGVGRVIWVRSSIPSHAASPTSWGTFRSSA

SEQ ID NO: 105 - protein, 99 aa, Artificial Sequence
  anti-HCV immunogenic peptide
GWVCARLGRLPSGRNLVEGDNLSPRLASPRAGPGLSPGTHGPSMATRAWGGQDGSCPPVALGLIGAPRTPGVGRVIWVRSSIPSHAASPTSWGTFRSSA

SEQ ID NO: 106 - protein, 99 aa, Artificial Sequence
  anti-HCV immunogenic peptide
GWVCARLGRLPSGLNLVEGDNLSPRLASPRVGPGLSPGTHGPSTATRAWGGQDGSCPPVAPGLAGAPRTPGVGRVIWVRSSIPSHAASPTSWGTFRSSA

SEQ ID NO: 107 - protein, 99 aa, Artificial Sequence
  anti-HCV immunogenic peptide
GWVCARLGRLPSGRNLVEGDNLSPRLASPRVGPGLSPGTLGPSTAMRAWDGRDGSCPPVVLGLVGAPRTPGVGRVIWVRSSIPSHAASPTSWGTFRSSA

SEQ ID NO: 108 - protein, 99 aa, Artificial Sequence
  anti-HCV immunogenic peptide
GWVCARLGRLPSGRNLVEGDNLSPRLASPRAGPGLSLGTLGPSMAMRVWDGQDGSCHPVALGLVGAPRTPGVGRVTWVRSSIPSHAASPTSWGTFRSSA

SEQ ID NO: 109 - protein, 99 aa, Artificial Sequence
  anti-HCV immunogenic peptide
GWVCARLGRLPSGRNLVEGDNLSPRLASPRAGPGLSLGTPGPSMAMRAWDGQDGSCPPAALGLVGAPLTPGVGRVIWVRSSIPSHAASPTSWGTFRSSA

SEQ ID NO: 110 - protein, 99 aa, Artificial Sequence
  anti-HCV immunogenic peptide
GWVCARLGRLPSGRNLVEGDNLSPRLASPRAGPGLSPGTLGPSTAMRVWGGQDGSCHPVALGLVGALRTPGVGRVIWVRSSIPSHAASPTSWGTSRSSA

SEQ ID NO: 111 - protein, 99 aa, Artificial Sequence
  anti-HCV immunogenic peptide
GWVCARLGRLPSGRNPVEGDNLSPRLASPRAGPGLSPGTLGPSTAMRVWGGQDGSCHPVALGLVGALRTPGVGRVIWVRSSIPSHAASPTSWGTSRSSA

SEQ ID NO: 112 - protein, 99 aa, Artificial Sequence
  anti-HCV immunogenic peptide
GWVCARLGRLPSGRNLVEGDNLSPRLAIPRVGPGLSPGTLGPSTAMRAWGGQDGSCHPVAPGLVGAPQTPGVGRVIWVRSSIPSHAASPTSWGTSRSSA

SEQ ID NO: 113 - protein, 99 aa, Artificial Sequence
  anti-HCV immunogenic peptide
GWVCARLGRLPSGRNLVEGDNLSPRLASPRVGPGLSPGTLGPSTPMRAWGGQDGSCHPVAPGLVGAPPTPGVGRVIWVRSSTPPHAASPTSWGTFRSSA

SEQ ID NO: 114 - protein, 99 aa, Artificial Sequence
  anti-HCV immunogenic peptide
GWVCAQLGRLPSGRNLVEGDNLSPRLAIPRAGPGLSPGTLGPSTAMRAWGGQDGSCHPVALGLVGAPMTPGVGRVIWVRSSIPSHAALPTSWGTFRSSA

SEQ ID NO: 115 - protein, 99 aa, Artificial Sequence
  anti-HCV immunogenic peptide
GWVCARLGRLPSGRNLVEGDNLSPRLASPRAGTGLSPGILGPSMAMRAWGGQDGSCHPVAPGLVGAPQTPGVGRVIWVRSSIPSHAASPTSWGTFRSSA

SEQ ID NO: 116 - protein, 99 aa, Artificial Sequence
  anti-HCV immunogenic peptide
GWVCARLGRLPSGRNLVEGDNLSPRLADPRAGTGLSPGILGPSMAMRAWGGQDGSCHPAAPGLVGAPQTPGVGRVIWVRSSIPSRAASPTSWGTFRSSA

SEQ ID NO: 117 - protein, 99 aa, Artificial Sequence
  anti-HCV immunogenic peptide
GWVCARLGRLPSGRNLVEGDNLSPRLASPRAGTGLSPGILGPSMAMRAWGGQDGSCHPAAPGLVGAPQTPGVGRVFWVRSSIPSHAASPTSWGTFRSSA

SEQ ID NO: 118 - protein, 99 aa, Artificial Sequence
  anti-HCV immunogenic peptide
GWVCARLGRLPSGRNLVEGDNLSPRLASPRAGPGLSPGTPGPSMAMRAWGGQDGSCHPAAPGLVGAPKTPGVGRVIWVRSSIPSHAASPTSWGTFRSSA

SEQ ID NO: 119 - protein, 99 aa, Artificial Sequence
  anti-HCV immunogenic peptide
GWVCARLGRLPSGRNLVEGDNLSPRLANPRAGSGLSPGTPGPSMAMRAWGGQDGSCHPAAPGLVGAPQTPGVGRVIWVRSSIPSHAASPTSWGTFRSSA

SEQ ID NO: 120 - protein, 99 aa, Artificial Sequence
  anti-HCV immunogenic peptide
GWVCARLGRLPSGRNLVEGDNLSPRLASPRVGPGLSPGTLGPSMAMRAWGGQDGSCPPAAPGLVGAPQTPGVGRVIWVRSSIPSHAASPTSWGTFRSSA

SEQ ID NO: 121 - protein, 99 aa, Artificial Sequence
  anti-HCV immunogenic peptide
GWVCARLGRLPSGRNLVEGDNLSPRLASPRAGPGLSPGTLGPSMAMRAWGGQDGSCHPAAPGLIGAPQTPGVGRVIWVRSSIPSHAASPTSWGTFRSSA

SEQ ID NO: 122 - protein, 99 aa, Artificial Sequence
  anti-HCV immunogenic peptide
GWVCARLGRLPSGRNLVEGDNLFPRLASPRAGPGLSPGTLGPSMAMRAWGGQDGSCHPVAPGLVGAPQTPGVGRVIWVRSSIPSHAASPTSWGTFRSSA

SEQ ID NO: 123 - protein, 99 aa, Artificial Sequence
  anti-HCV immunogenic peptide
GWVCARLGRLPSGRNLVEGDNLSPRLASPRAGPGLSPGTLGPSMAMRVWGGQDGSCHPVAPGLVGAPRTPGVGRVIWVRSSIPSHAASPTSWGTFRSSA

SEQ ID NO: 124 - protein, 99 aa, Artificial Sequence
  anti-HCV immunogenic peptide
GWVCARLGRLPSGRNLVEGDNLSPRLASPRAGPGLSPGTLGPSMATRVWGGQDGSCHPVAPGLVGAPRTPGVGRVIWVRSSIPSHAASPTSWGTFRSSA

SEQ ID NO: 125 - protein, 99 aa, Artificial Sequence
  anti-HCV immunogenic peptide
GWVCARPGRLPSGRNLVEGDNLSPRLASPRAGPGLSPGTLGPSMAMRVWGGQDGSCHPTAPGLGGAPRTPGVGRVIWVRSSIPSHAASPTSWGTFRSSG

SEQ ID NO: 126 - protein, 99 aa, Artificial Sequence
  anti-HCV immunogenic peptide
GWVCARLGRLPSGRNLVEGDNLSPRLASPRAGPGLSPGTLGPSMAMRVWGGQDGSCHPTAPGLGGAPRTPGVGRVIWVRSSIPSHAASPTSWGTFRSSA

SEQ ID NO: 127 - protein, 99 aa, Artificial Sequence
  anti-HCV immunogenic peptide
GWVCARLGRLPSGRNLVEGDNLSPRLASPRVGPGLSPGTLGPSMAMRVWGGQDGSCHPTAPGLGGAPRTPGVGRVIWVRSSIPSHAASPTSWGTFRSSA

SEQ ID NO: 128 - protein, 99 aa, Artificial Sequence
  anti-HCV immunogenic peptide
GWVCARLGRLPSGRNLVEGDNLSPRLASPRAGPGLSPGTLGPSMAMRAWGGQDGSCHPAALGLVGAPRTPGVGRVIWVRSSIPLHAASPTSWGTSRSSA

SEQ ID NO: 129 - protein, 99 aa, Artificial Sequence
  anti-HCV immunogenic peptide
GWVCARLGRLPSGHNLVAGDNLSPRLASPRAGPGLSPGTLGPSMATRAWGGQDGSCHPVALGLVGAPQTPGVGRVIWVRSSIPLHAASPTSWGTSRSSA

SEQ ID NO: 130 - protein, 99 aa, Artificial Sequence
  anti-HCV immunogenic peptide
GWVCARLGRLPSGRNLVEGDNLSPRLAGPRAGPGLSPGTLGPSMAMRAWGGQDGSCHPVAPGLVGAPRTPGVGRAIWVRSSIPLHAASPTSWGTSRSSV

SEQ ID NO: 131 - protein, 99 aa, Artificial Sequence
  anti-HCV immunogenic peptide
GWVCARPGRLPSGRNLVEGDNLSPRLANPRAGPGLSPGTLGPSMAMRVWGGQDGSCHPVVLGLVGAPRTPGVGRVIWVRSSIPLHAASPTSWGTFRSSA

SEQ ID NO: 132 - protein, 99 aa, Artificial Sequence
  anti-HCV immunogenic peptide
GWVCARSGRLPSGRNLVEGDNLSPRLADPRAGPGLSPGTLGPSMAMRAWGGQDGSCHPVVLGLVGAPQTPGVGRVIWVRSSIPLHAASPTSWGTFRSSA

SEQ ID NO: 133 - protein, 99 aa, Artificial Sequence
  anti-HCV immunogenic peptide
GWVCARLGRLPSGHNLVEGDNLSPRLAVPRAGPGLSPGTLGPSMAMRAWGGQDGSCHPEAPGLIGAPRTPGVGRVIWVRSSIPSHAASPTSWGTFRSSA

SEQ ID NO: 134 - protein, 99 aa, Artificial Sequence
  anti-HCV immunogenic peptide
GWVCARLGRLPSGRNLVEGDNLSPRLANPRAGPGLSPGTLGPSMPMRAWGGQDGSCHPEAPGLIGAPRTPGVGRVIWVRSSIPLHAASPTSWGTFRSSA

SEQ ID NO: 135 - protein, 99 aa, Artificial Sequence
  anti-HCV immunogenic peptide
GWVCARLGRLPSGRNLVEGDNLSPRLANPRAGPGLSPGTLGPSMAMRAWGGQDGSCHPVALGLIGAPRTPGVGRVIWVRSSIPSHAASPTSWGTFRSSA

SEQ ID NO: 136 - protein, 99 aa, Artificial Sequence
  anti-HCV immunogenic peptide
GWVCARLGRLPSGRNLVEGDNLSPRLAIPRAGPGLSLGTLGPSMAMRAWGGQDGSCHPVALGLVGAPRTPGVGRAIWVRSSIPSRAASPTSWGTFRSSA

SEQ ID NO: 137 - protein, 99 aa, Artificial Sequence
  anti-HCV immunogenic peptide
GWVCARLGRLPSGRNLVEGDNLSPRLAVPRAGPGLSPGTLGPSMAMRAWGGQDGSCHPVALGLVGAPRTPGVGRAIWVRSSIPSRAASPTSWGTFRLSA

SEQ ID NO: 138 - protein, 99 aa, Artificial Sequence
  anti-HCV immunogenic peptide
GWVCARLGRLPSGRNLVEGDNLSPRLAGPRAGPGLSPGTLGPSMAMRAWGGQDGSCHPVVLGLVGAPRTPGVGRAIWVRSSIPSRAASPTSWGTFRSSA

SEQ ID NO: 139 - protein, 99 aa, Artificial Sequence
  anti-HCV immunogenic peptide
GWVCARLGRLPSGHNLVDGDNLSPRLAGPRAGPGPSPGILGPSMAMRAWGGQDGSCHPVAPGLVGAPRTPGVGRAIWVRSSIPSRAASPTSWGTFRSSA

SEQ ID NO: 140 - protein, 99 aa, Artificial Sequence
  anti-HCV immunogenic peptide
GWVCARLGRLPSGRNLVEGDNLSPRLASPRAGPGLSRGILGPSMATRAWGGQDGSCHPVALGLVGAPRTPGVGRVIWVRSSIPSRAASPTSWGTFRSSV

SEQ ID NO: 141 - protein, 99 aa, Artificial Sequence
  anti-HCV immunogenic peptide
GWVCARLGRLPSGRNLVEGDNLSPRLASPRAGPGPSPGILGPSMATRAWGGQDGSCHPVALGLAGAPRTPGVGRVIWVRSSIPSRAASPTSWGTFRSSA

SEQ ID NO: 142 - protein, 99 aa, Artificial Sequence
  anti-HCV immunogenic peptide
GWVCARPGRLPSGRNLVEGDNLSPRLADPRAGPGLSPGILGPSMAMRAWGGQDGSCHPMALGLVGAPRTPGVGRVIWVRSSIPSRAASPTSWGTSRSSA

SEQ ID NO: 143 - protein, 99 aa, Artificial Sequence
  anti-HCV immunogenic peptide
GWVCARPGRLPSGRNLVEGDNLSPRLANPRAGPGLSPGILGPSMATRAWGGQDGSCHPAALGLIGAPRTPGVGRAIWVRSSIPSRAASPTSWGTSRSSV

SEQ ID NO: 144 - protein, 99 aa, Artificial Sequence
  anti-HCV immunogenic peptide
GWVCARLGRLPSGRNLVEGDNLSPRLASPRVGPGLSPGTPGPSMAMRAWGGQDGSCHPVALGLVGAPRTPGVGRAIWVRSSIPSRAASPISWGTFRSSA

SEQ ID NO: 145 - protein, 99 aa, Artificial Sequence
  anti-HCV immunogenic peptide
GWVCARLGRLPSGRNLVEGDNLSPRLASPRVGPGLSPGTHGPSMAMRVWGGQDGSCHPVDPGLVGAPRTPGVGRAIWVRSSIPSHAASPTSWGTFRSSA

SEQ ID NO: 146 - protein, 99 aa, Artificial Sequence
  anti-HCV immunogenic peptide
GWVCARLGRLPSGRNLVAGDNLSPRLAGPRVGPGLSPGTLGPSMPMRAWGGRDGSCHPAAPGLVGAPRTPGVGRAIWVRSSIPSHAASPTSWGTFRSSA

SEQ ID NO: 147 - protein, 99 aa, Artificial Sequence
  anti-HCV immunogenic peptide
GWVCARLGRLPSGRNLVEGDNLSPRLASPRAGPGLSPGTLGPSMAMRAWGGQDGSCHPAAPGLVGAPPTPGVGRAIWVRSSIPSRVASPTSWGTFRSSA

SEQ ID NO: 148 - protein, 99 aa, Artificial Sequence
  anti-HCV immunogenic peptide
RWVCARLGRLPSGRNLVEGDNLSPRLADPRVGPGLSLGTLGPSMAMRAWGGQDGSCHPAAPGLVGAPRTPGAGRAIWVRSSIPSRAASPTSWGTFRSSA

SEQ ID NO: 149 - protein, 99 aa, Artificial Sequence
  anti-HCV immunogenic peptide
RWVCARLGRLPSGRNLVEGDNLSPRLADPRVGPGLSLGTLGPSMAMRAWGGQDGSCHPAAPGLVGAPRTPGGGRAIWVRSSIPSRAASPTSWGTFRSSA

SEQ ID NO: 150 - protein, 99 aa, Artificial Sequence
  anti-HCV immunogenic peptide
SWVCARLGRLPSGRNLVGGDNLSPRLANPRVGPGLSPGTLGPSMATRAWGGQDGSCHPAAPGLVGAPQTPGVGHAIWVRSSIPSRVASPTSWGTFRSSA

SEQ ID NO: 151 - protein, 99 aa, Artificial Sequence
  anti-HCV immunogenic peptide
  MISC_FEATURE (1)..(1)
  MISC_FEATURE (8)..(8): V, A or T
  MISC_FEATURE (9)..(9): K, R or N
  MISC_FEATURE (11)..(11): L, P or Q
  MISC_FEATURE (12)..(12): S or N
  MISC_FEATURE (13)..(13): G or D
  MISC_FEATURE (14)..(14): H, R or Q
  MISC_FEATURE (15)..(15): S or N
  MISC_FEATURE (16)..(16): L or P
  MISC_FEATURE (18)..(18): D, V, N, R or T
  MISC_FEATURE (19)..(19): G, D or S
  MISC_FEATURE (20)..(20): D, V, A, G or E
  MISC_FEATURE (21)..(21): S, N or T
  MISC_FEATURE (22)..(22): L or P
  MISC_FEATURE (23)..(23): S, P or F
  MISC_FEATURE (25)..(25): R or G
  MISC_FEATURE (26)..(26): R, H or L
  MISC_FEATURE (27)..(27): V or A
  MISC_FEATURE (28)..(28): G, R, E, H or V
  MISC_FEATURE (29)..(29): A, P or L
  MISC_FEATURE (30)..(30): R, K, E or T
  MISC_FEATURE (31)..(31): A or D
  MISC_FEATURE (32)..(32): G or D
  MISC_FEATURE (35)..(35): L, P or R
  MISC_FEATURE (37)..(37): P or L
  MISC_FEATURE (40)..(40): L or P
  MISC_FEATURE (42)..(42): P or L
  MISC_FEATURE (43)..(43): S, Y or F
  MISC_FEATURE (44)..(44): M or T
  MISC_FEATURE (45)..(45): V, G, A or E
  MISC_FEATURE (46)..(46): M, T or I
  MISC_FEATURE (49)..(49): A or V
  MISC_FEATURE (50)..(50): G, V or D
  MISC_FEATURE (51)..(51): G or W
  MISC_FEATURE (52)..(52): Q or R
  MISC_FEATURE (53)..(53): G or D
  MISC_FEATURE (55)..(55): S or F
  MISC_FEATURE (57)..(57): P or L
  MISC_FEATURE (58)..(58): P, H, R or L
  MISC_FEATURE (59)..(59): A or V
  MISC_FEATURE (60)..(60): A or V
  MISC_FEATURE (61)..(61): P or L
  MISC_FEATURE (62)..(62): V, A, D or G
  MISC_FEATURE (63)..(63): H, L, P, Q or R
  MISC_FEATURE (64)..(64): L, A, V, R, I or P
  MISC_FEATURE (66)..(66): A or P
  MISC_FEATURE (67)..(67): Q, K or P
  MISC_FEATURE (68)..(68): M or T
  MISC_FEATURE (72)..(72): V, G, D, E or A
  MISC_FEATURE (73)..(73): G or D
  MISC_FEATURE (74)..(74): P, H or L
  MISC_FEATURE (76)..(76): I or T
  MISC_FEATURE (77)..(77): W or L
  MISC_FEATURE (78)..(78): V or A
  MISC_FEATURE (79)..(79): R or K
  MISC_FEATURE (82)..(82): I or T
  MISC_FEATURE (83)..(83): P or L
  MISC_FEATURE (84)..(84): S or L
  MISC_FEATURE (85)..(85): R or H
  MISC_FEATURE (86)..(86): A or V
  MISC_FEATURE (87)..(87): D, G, A or V
  MISC_FEATURE (88)..(88): S or L
  MISC_FEATURE (89)..(89): P or L
  MISC_FEATURE (90)..(90): T, I or A
  MISC_FEATURE (94)..(94): T or I
  MISC_FEATURE (95)..(95): F or S
  MISC_FEATURE (98)..(98): S, L or Q
  MISC_FEATURE (99)..(99): A or V
XWVCXXXXXLXXXXXXAXXXXXXPXXXXXXXXPGXSXGTXGXXXXXRAXXXXXGXCXXXXXXXXGXXXTPGXXXAXXXXSSXXXXXXXXXSWGXXRSXX

SEQ ID NO: 152 - protein, 99 aa, Artificial Sequence
  anti-HCV immunogenic peptide
DWVCARRVKLLSGHSLADGDSLSPRRVGARAGPGLSPGTLGPSMVMRAAGGQGGSCPPAAPVHLGAQMTPGVGPAIWVRSSIPLRADSPTSWGTFRSSA

SEQ ID NO: 153 - protein, 99 aa, Artificial Sequence
  anti-HCV immunogenic peptide
DWVCARRVKLLNGHSLADDDSLSPRRVGAKAGPGLSPGTLGPSMVTRAAGGQGGSCPHAAPVHPGAQMTPGGGPAIWVKSSIPLRADSPTSWGTSRSSA

SEQ ID NO: 154 - protein, 99 aa, Artificial Sequence
  anti-HCV immunogenic peptide
DWVCARPVKLLNGHSLADDDSLSPRRVGATAGPGLSPGTLGPSMVTRAAGGQGGSCPHAALVHLGAQMTPGGGPAIWVKSSIPLRADSPTSWGTSRSSA

SEQ ID NO: 155 - protein, 99 aa, Artificial Sequence
  anti-HCV immunogenic peptide
DWVCARRVKLLNGHSPADDDSLSPRRVRAEAGPGLSPGTLGPSMVMRAAGGQGGSCPRAAPVHLGAQMTPGGGPAIWVKSSIPLRVDSPTSWGTSRSSA

SEQ ID NO: 156 - protein, 99 aa, Artificial Sequence
  anti-HCV immunogenic peptide
DWVCARRVKLLNGRSLADDDSLSPRRVGAKAGPGLSPGTLGPSMVTRAAGGQDGSCPHAAPVHLGAQTTPGDGPAIWVKSSIPLRADSPTSWGTSRSSA

SEQ ID NO: 157 - protein, 99 aa, Artificial Sequence
  anti-HCV immunogenic peptide
DWVCARRVKLLNGHSLADDGSLSPRRVGAKAGPGLSPGTLGPSMVTRAAGGQDGSCPRAAPVHLGAQMTPGDGPAIWVKSSIPLRADSPTSWGTSRSSA

SEQ ID NO: 158 - protein, 99 aa, Artificial Sequence
  anti-HCV immunogenic peptide
DWVCARRVKLLNGHSLADDDSLSPRHVGAKAGPGLSLGTLGPSMVTRAAGGQGGSCPHAAPVHLGAQTTPGDGPAIWVKSSIPLRADSPTSWGTSRSSA

SEQ ID NO: 159 - protein, 99 aa, Artificial Sequence
  anti-HCV immunogenic peptide
DWVCARRVKLLNGHSLADDDSLSPRRVGAKAGPGLSPGTLGPSMVTRAAGGQGGSCPHAAPVQLGAQTTPGGGPAIWVKSSIPLRAVSPTSWGTSRSSA

SEQ ID NO: 160 - protein, 99 aa, Artificial Sequence
  anti-HCV immunogenic peptide
DWVCARRVKLLNGHSLADDDSLSPRRVGAKAGPGLSPGTLGPSMVMRAAGGQGGSCPHAAPVHLGAQTTPGGGPAIWVKSSIPLRADSPTSWGTSRSSA

SEQ ID NO: 161 - protein, 99 aa, Artificial Sequence
  anti-HCV immunogenic peptide
DWVCARRVKLLNGHSLADGDSLSPRRVGAKADPGLSPGTLGPSMVTRAAGGQGGSCPHAAPVHLGAQMTPGGGPAIWVKSSIPLRAASPTSWGTSRSSA

SEQ ID NO: 162 - protein, 99 aa, Artificial Sequence
  anti-HCV immunogenic peptide
DWVCARRVKLLNGHSLADGDSLSPRRVGAKAGPGLSPGTLGPSMVTRAAGGQGGSCPRAALVHLGAKMTPGGGPATWARSSIPLRADSPTSWGTSRSSA

SEQ ID NO: 163 - protein, 99 aa, Artificial Sequence
  anti-HCV immunogenic peptide
DWVCARRVKLLNGHSLADGDSLSPRRVGAKAGPGLSLGTLGPSMVTRAAGGQGGSCPHAAPVHLGAQTTPGGGPAIWVRSSIPSRADSPTSWGTSRSSA

SEQ ID NO: 164 - protein, 99 aa, Artificial Sequence
  anti-HCV immunogenic peptide
DWVCARRVKLLNGRSLADGESLSPRRVGAKAGPGPSLGTLGPSMVMRAAGGQGGSCPHAAPVQIGPQMTPGDGPAIWVKSSIPSRADSPISWGTSRSSA

SEQ ID NO: 165 - protein, 99 aa, Artificial Sequence
  anti-HCV immunogenic peptide
DWVCAQLVRLQSGHNPADGDSLFPRHARAEDGPGRSLGTPGPSMVTRAAGGQGGFCLLAAPDRVGAPTTPGEGPAIWVRSSTLSHAGSPISWGIFRSSA

SEQ ID NO: 166 - protein, 99 aa, Artificial Sequence
  anti-HCV immunogenic peptide
DWVCAQFVKLPSGQNPATGGSLSPRHVGARAGPGLSLGTLGLFMAMRAVGGQDGSCPPAALGHLGAPMTPGEGLATWVKSSIPLRADSPISWGTFRSSA

SEQ ID NO: 167 - protein, 99 aa, Artificial Sequence
  anti-HCV immunogenic peptide
YWVCALHVRLPSGHSLARGGSLSPRRVVARDGPGLSPGTLGPSTVMRAAGGRDGSCPPAVLGRAGAQMTPGEDPAIWVRSSIPLHAGSPTSWGTFRSSA

SEQ ID NO: 168 - protein, 99 aa, Artificial Sequence
  anti-HCV immunogenic peptide
DWVCAQYVRLPSGRNLADGVSLSPRHARARAGPGLSLGTLGPSTEMRAVGGQDGFCPPAVLVLIGAQMTPGVDPATWVRSSTPLHADSPTSWGTFRSSA

SEQ ID NO: 169 - protein, 99 aa, Artificial Sequence
  anti-HCV immunogenic peptide
DWVCAQYVRLPSGRSLANSVTLSPRLARARAGPGLSPGTLGPSTGIRAVGWQDGSCPPVVLALVGAQMTPGVDPATLVRSSIPLRVDSPTSWGTFRSSA

SEQ ID NO: 170 - protein, 99 aa, Artificial Sequence
  anti-HCV immunogenic peptide
NWVCAKYVRLPSDHNLADGVSLPPRHARARAGPGPSPGTLGPSTGMRAVVGQDGSCPPAVLAPAGAKMTPGVDPATWVRSSIPLRADLPTSWGTFRSSA

SEQ ID NO: 171 - protein, 99 aa, Artificial Sequence
  anti-HCV immunogenic peptide
DWVCAQYVKLPSGRNLADGVSLSPRHAHARAGPGLSLGTLGPSMGMRAAVGQDGFCPPVVLALVGAQMTPGVDPATWVKSSIPLRADSPTSWGTFRSSA

SEQ ID NO: 172 - protein, 99 aa, Artificial Sequence
  anti-HCV immunogenic peptide
DWVCAQCVNLPSGRNLADGGSLSPRHAEARAGPGPSLGTLGPSMGMRAVGGQDGSCLPVAPALVGAQMTPGVDHATWVRSSIPSRVDSPISWGTFRSLV

SEQ ID NO: 173 - protein, 99 aa, Artificial Sequence
  anti-HCV immunogenic peptide
DWVCAQYVRLPSGHNPADGASLSPRRAHARAGPGLSLGTPGPFMGTRAAVGQDGSCPPAVPDPAGAQMTPGADPATWVRSSIPLRADSLTSWGIFRSSA

SEQ ID NO: 174 - protein, 99 aa, Artificial Sequence
  anti-HCV immunogenic peptide
SWVCVQCARLPSGRNLAVGANPSPGRAEPRAGPGLSPGTLGPYMGMRAAGGQGGSCPRAALAHRGAQMTPGVDHAIWVRSSIPSRADSPTSWGIFRSSA

SEQ ID NO: 175 - protein, 99 aa, Artificial Sequence
  anti-HCV immunogenic peptide
GWVCVQCTRLPSGRNLAVGANPSPGRAELRAGPGLSPGTLGPYMGMRAAGGQDGSCPRAALAHRGAQMTPGVDPAIWVRSSIPSHADSPASWGTFRSSA

SEQ ID NO: 176 - protein, 99 aa, Artificial Sequence
  anti-HCV immunogenic peptide
SWVCVRCARLPSGHNLAVGASPSPGRAEPRAGPGLSPGTLGPYMGMRAADGQGGSCPPAALAHRGAQMTPGVDPAIWVRSSTPSHADSPTSWGTFRSQA

SEQ ID NO: 177 - protein, 9 aa, Artificial Sequence
  anti-HCV immunogenic peptide
APGWVCARL

SEQ ID NO: 178 - protein, 9 aa, Artificial Sequence
  anti-HCV immunogenic peptide
ALDWVCARR

SEQ ID NO: 179 - protein, 9 aa, Artificial Sequence
  anti-HCV immunogenic peptide
APGWVCARQ

SEQ ID NO: 180 - protein, 9 aa, Artificial Sequence
  anti-HCV immunogenic peptide
AHVWVCARR

SEQ ID NO: 181 - protein, 9 aa, Artificial Sequence
  anti-HCV immunogenic peptide
AQLWVCVQC

SEQ ID NO: 182 - protein, 9 aa, Artificial Sequence
  anti-HCV immunogenic peptide
AQLWVCVRR

SEQ ID NO: 183 - protein, 9 aa, Artificial Sequence
  anti-HCV immunogenic peptide
APGWVCARP

SEQ ID NO: 184 - protein, 9 aa, Artificial Sequence
  anti-HCV immunogenic peptide
APSWVCARL

SEQ ID NO: 185 - protein, 9 aa, Artificial Sequence
  anti-HCV immunogenic peptide
APGWVCAHL

SEQ ID NO: 186 - protein, 9 aa, Artificial Sequence
  anti-HCV immunogenic peptide
APGWVCAQL

SEQ ID NO: 187 - protein, 9 aa, Artificial Sequence
  anti-HCV immunogenic peptide
ALGWVCARL

SEQ ID NO: 188 - protein, 9 aa, Artificial sequence
  anti-HCV immunogenic peptide
APGWVCVRL

SEQ ID NO: 189 - protein, 9 aa, Artificial Sequence
  anti-HCV immunogenic peptide
APVWVCARL

SEQ ID NO: 190 - protein, 9 aa, Artificial Sequence
  anti-HCV immunogenic peptide
AQGWVCARR

SEQ ID NO: 191 - protein, 9 aa, Artificial Sequence
  anti-HCV immunogenic peptide
APGWVCARS

SEQ ID NO: 192 - protein, 9 aa, Artificial Sequence
  anti-HCV immunogenic peptide
APRWVCARL

SEQ ID NO: 193 - protein, 9 aa, Artificial Sequence
  anti-HCV immunogenic peptide
APEWVCARR

SEQ ID NO: 194 - protein, 9 aa, Artificial Sequence
  anti-HCV immunogenic peptide
AHDWVCAQC

SEQ ID NO: 195 - protein, 9 aa, Artificial Sequence
  anti-HCV immunogenic peptide
AHDWVCAQL

SEQ ID NO: 196 - protein, 9 aa, Artificial Sequence
  anti-HCV immunogenic peptide
GHDWVCARR

SEQ ID NO: 197 - protein, 9 aa, Artificial Sequence
  anti-HCV immunogenic peptide
AHDWVCARR

SEQ ID NO: 198 - protein, 9 aa, Artificial Sequence
  anti-HCV immunogenic peptide
ARDWVCARR

SEQ ID NO: 199 - protein, 9 aa, Artificial Sequence
  anti-HCV immunogenic peptide
AQDWVCARR

SEQ ID NO: 200 - protein, 9 aa, Artificial Sequence
  anti-HCV immunogenic peptide
AHDWVCARP

SEQ ID NO: 201 - protein, 9 aa, Artificial Sequence
  anti-HCV immunogenic peptide
APGWVCARR

SEQ ID NO: 202 - protein, 9 aa, Artificial Sequence
  anti-HCV immunogenic peptide
VPEWVCARR

SEQ ID NO: 203 - protein, 9 aa, Artificial Sequence
  anti-HCV immunogenic peptide
ALYWVCALH

SEQ ID NO: 204 - protein, 9 aa, Artificial Sequence
  anti-HCV immunogenic peptide
ALGWVCARR

SEQ ID NO: 205 - protein, 9 aa, Artificial Sequence
  anti-HCV immunogenic peptide
APGWVCAQR

SEQ ID NO: 206 - protein, 9 aa, Artificial Sequence
  anti-HCV immunogenic peptide
APSWVCVQC

SEQ ID NO: 207 - protein, 9 aa, Artificial Sequence
  anti-HCV immunogenic peptide
ALDWVCARL

SEQ ID NO: 208 - protein, 9 aa, Artificial Sequence
  anti-HCV immunogenic peptide
APSWVCVQY

SEQ ID NO: 209 - protein, 9 aa, Artificial Sequence
  anti-HCV immunogenic peptide
ARVWVCARR

SEQ ID NO: 210 - protein, 9 aa, Artificial Sequence
  anti-HCV immunogenic peptide
APGWVCVQC

SEQ ID NO: 211 - protein, 9 aa, Artificial Sequence
  anti-HCV immunogenic peptide
ARAWVCARR

SEQ ID NO: 212 - protein, 9 aa, Artificial Sequence
  anti-HCV immunogenic peptide
ALVWVCARR

SEQ ID NO: 213 - protein, 9 aa, Artificial Sequence
  anti-HCV immunogenic peptide
ARVWVCARQ

SEQ ID NO: 214 - protein, 9 aa, Artificial Sequence
  anti-HCV immunogenic peptide
ALGWVCARL

SEQ ID NO: 215 - protein, 9 aa, Artificial Sequence
  anti-HCV immunogenic peptide
APDWVCAQL

SEQ ID NO: 216 - protein, 9 aa, Artificial Sequence
  anti-HCV immunogenic peptide
APDWVCARL

SEQ ID NO: 217 - protein, 9 aa, Artificial Sequence
  anti-HCV immunogenic peptide
ALGWVCAQL

SEQ ID NO: 218 - protein, 9 aa, Artificial Sequence
  anti-HCV immunogenic peptide
AHGWVCARR

SEQ ID NO: 219 - protein, 9 aa, Artificial Sequence
  anti-HCV immunogenic peptide
AHDWVCAPQ

SEQ ID NO: 220 - protein, 9 aa, Artificial Sequence
  anti-HCV immunogenic peptide
APGWVCVRQ

SEQ ID NO: 221 - protein, 9 aa, Artificial Sequence
  anti-HCV immunogenic peptide
AHNWVCAKY

SEQ ID NO: 222 - protein, 9 aa, Artificial Sequence
  anti-HCV immunogenic peptide
ALDWVCARR

SEQ ID NO: 223 - protein, 9 aa, Artificial Sequence
  anti-HCV immunogenic peptide
ALDWVCAQR

SEQ ID NO: 224 - protein, 9 aa, Artificial Sequence
  anti-HCV immunogenic peptide
APGWVCAQQ

SEQ ID NO: 225 - protein, 9 aa, Artificial Sequence
  anti-HCV immunogenic peptide
APSWVCVCR

SEQ ID NO: 226 - protein, 9 aa, Artificial Sequence
  anti-HCV immunogenic peptide
AQGWVCARL

SEQ ID NO: 227 - protein, 9 aa, Artificial Sequence
  anti-HCV immunogenic peptide
DLGWVCARP

SEQ ID NO: 228 - protein, 9 aa, Artificial Sequence
  anti-HCV immunogenic peptide
ALVWVCARL

SEQ ID NO: 229 - protein, 9 aa, Artificial Sequence
  anti-HCV immunogenic peptide
AHDWVCAQY

SEQ ID NO: 230 - protein, 9 aa, Artificial Sequence
  anti-HCV immunogenic peptide
ALNWVCARL

SEQ ID NO: 231 - protein, 9 aa, Artificial Sequence
  anti-HCV immunogenic peptide
ARDWVCAQR

SEQ ID NO: 232 - protein, 9 aa, Artificial Sequence
  anti-HCV immunogenic peptide
APDWVCARQ

SEQ ID NO: 233 - protein, 9 aa, Artificial Sequence
  anti-HCV immunogenic peptide
ARDWVCAQY

SEQ ID NO: 234 - protein, 9 aa, Artificial Sequence
  anti-HCV immunogenic peptide
AQDWVCAQF

SEQ ID NO: 235 - protein, 9 aa, Artificial Sequence
  anti-HCV immunogenic peptide
APGWVCARG

SEQ ID NO: 236 - protein, 9 aa, Artificial Sequence
  anti-HCV immunogenic peptide
WVCARLGRL

SEQ ID NO: 237 - protein, 9 aa, Artificial Sequence
  anti-HCV immunogenic peptide
WVCARQGRL

SEQ ID NO: 238 - protein, 9 aa, Artificial Sequence
  anti-HCV immunogenic peptide
WVCARQGSL

SEQ ID NO: 239 - protein, 9 aa, Artificial Sequence
  anti-HCV immunogenic peptide
WVCARRGKP

SEQ ID NO: 240 - protein, 9 aa, Artificial Sequence
  anti-HCV immunogenic peptide
WVCARRGKL

SEQ ID NO: 241 - protein, 9 aa, Artificial Sequence
  anti-HCV immunogenic peptide
WVCARGGRL

SEQ ID NO: 242 - protein, 9 aa, Artificial Sequence
  anti-HCV immunogenic peptide
WVCAQRGKL

SEQ ID NO: 243 - protein, 9 aa, Artificial Sequence
  anti-HCV immunogenic peptide
WVCAQRGRL

SEQ ID NO: 244 - protein, 9 aa, Artificial Sequence
  anti-HCV immunogenic peptide
WVCARQGIL

SEQ ID NO: 245 - protein, 9 aa, Artificial Sequence
  anti-HCV immunogenic peptide
WVCARQGKL

SEQ ID NO: 246 - protein, 9 aa, Artificial Sequence
  anti-HCV immunogenic peptide
WVCVRQGRL

SEQ ID NO: 247 - protein, 9 aa, Artificial Sequence
  anti-HCV immunogenic peptide
WVCARRERL

SEQ ID NO: 248 - protein, 9 aa, Artificial Sequence
  anti-HCV immunogenic peptide
WVCARPGRL

SEQ ID NO: 249 - protein, 9 aa, Artificial Sequence
  anti-HCV immunogenic peptide
WVCARRGRL

SEQ ID NO: 250 - protein, 9 aa, Artificial Sequence
  anti-HCV immunogenic peptide
WVCVRLGRL

SEQ ID NO: 251 - protein, 9 aa, Artificial Sequence
  anti-HCV immunogenic peptide
WVCARLGRP

SEQ ID NO: 252 - protein, 9 aa, Artificial Sequence
  anti-HCV immunogenic peptide
WVCARLGKL

SEQ ID NO: 253 - protein, 9 aa, Artificial Sequence
  anti-HCV immunogenic peptide
WVSARGGRF

SEQ ID NO: 254 - protein, 9 aa, Artificial Sequence
  anti-HCV immunogenic peptide
WVVARLGRL

SEQ ID NO: 255 - protein, 9 aa, Artificial Sequence
  anti-HCV immunogenic peptide
WVCARSGRL

SEQ ID NO: 256 - protein, 9 aa, Artificial Sequence
  anti-HCV immunogenic peptide
WVCAHLGRL

SEQ ID NO: 257 - protein, 9 aa, Artificial Sequence
  anti-HCV immunogenic peptide
WLCARLGRL

SEQ ID NO: 258 - protein, 9 aa, Artificial Sequence
  anti-HCV immunogenic peptide
WACARLGRL

SEQ ID NO: 259 - protein, 9 aa, Artificial Sequence
  anti-HCV immunogenic peptide
WVCAQLGRL

SEQ ID NO: 260 - protein, 9 aa, Artificial Sequence
  anti-HCV immunogenic peptide
WVCARLERL

SEQ ID NO: 261 - protein, 9 aa, Artificial Sequence
  anti-HCV immunogenic peptide
WVCVQCVRL

SEQ ID NO: 262 - protein, 9 aa, Artificial Sequence
  anti-HCV immunogenic peptide
WVCVRRVRL

SEQ ID NO: 263 - protein, 9 aa, Artificial Sequence
  anti-HCV immunogenic peptide
WVCAQYVKL

SEQ ID NO: 264 - protein, 9 aa, Artificial Sequence
  anti-HCV immunogenic peptide
WVCAQYVRL

SEQ ID NO: 265 - protein, 9 aa, Artificial Sequence
  anti-HCV immunogenic peptide
WVCAKYVRL

SEQ ID NO: 266 - protein, 9 aa, Artificial Sequence
  anti-HCV immunogenic peptide
WVCAQCVNL

SEQ ID NO: 267 - protein, 9 aa, Artificial Sequence
  anti-HCV immunogenic peptide
WVCARRVKL

SEQ ID NO: 268 - protein, 9 aa, Artificial Sequence
  anti-HCV immunogenic peptide
WVCAQFVKL

SEQ ID NO: 269 - protein, 9 aa, Artificial Sequence
  anti-HCV immunogenic peptide
WVCAQLVRL

SEQ ID NO: 270 - protein, 9 aa, Artificial Sequence
  anti-HCV immunogenic peptide
WVCALHVRL

SEQ ID NO: 271 - protein, 9 aa, Artificial Sequence
  anti-HCV immunogenic peptide
WVCAVQCAL

SEQ ID NO: 272 - protein, 9 aa, Artificial Sequence
  anti-HCV immunogenic peptide
WVCAVQYAL

SEQ ID NO: 273 - protein, 9 aa, Artificial Sequence
  anti-HCV immunogenic peptide
WVCAVQCTL

SEQ ID NO: 274 - protein, 9 aa, Artificial Sequence
  anti-HCV immunogenic peptide
WVCAVLCAL

SEQ ID NO: 275 - protein, 9 aa, Artificial Sequence
  anti-HCV immunogenic peptide
WVCAQQGRL

SEQ ID NO: 276 - protein, 9 aa, Artificial Sequence
  anti-HCV immunogenic peptide
WVCARREKL

SEQ ID NO: 277 - protein, 9 aa, Artificial Sequence
  anti-HCV immunogenic peptide
WVCARQGRP

SEQ ID NO: 278 - protein, 9 aa, Artificial Sequence
  anti-HCV immunogenic peptide
WVCARRERP

SEQ ID NO: 279 - protein, 9 aa, Artificial Sequence
  anti-HCV immunogenic peptide
WVCARRGRP

SEQ ID NO: 280 - protein, 9 aa, Artificial Sequence
  anti-HCV immunogenic peptide
WVCAQRGRP

SEQ ID NO: 281 - protein, 9 aa, Artificial Sequence
  anti-HCV immunogenic peptide
WVCAQRERL

SEQ ID NO: 282 - protein, 9 aa, Artificial Sequence
  anti-HCV immunogenic peptide
WVCARQERL

SEQ ID NO: 283 - protein, 9 aa, Artificial Sequence
  anti-HCV immunogenic peptide
WVCAPQERH

SEQ ID NO: 284 - protein, 9 aa, Artificial Sequence
  anti-HCV immunogenic peptide
RLPSGRNLV

SEQ ID NO: 285 - protein, 9 aa, Artificial Sequence
  anti-HCV immunogenic peptide
RLPSGRNLE

SEQ ID NO: 286 - protein, 9 aa, Artificial Sequence
  anti-HCV immunogenic peptide
RLPSGRSLE

SEQ ID NO: 287 - protein, 9 aa, Artificial Sequence
  anti-HCV immunogenic peptide
RLPSGCNLE

SEQ ID NO: 288 - protein, 9 aa, Artificial Sequence
  anti-HCV immunogenic peptide
RLPSGHNLE

SEQ ID NO: 289 - protein, 9 aa, Artificial Sequence
  anti-HCV immunogenic peptide
RLPSARNHE

SEQ ID NO: 290 - protein, 9 aa, Artificial Sequence
  anti-HCV immunogenic peptide
RLQSGRNLV

SEQ ID NO: 291 - protein, 9 aa, Artificial Sequence
  anti-HCV immunogenic peptide
RLPSGRNLL

SEQ ID NO: 292 - protein, 9 aa, Artificial Sequence
  anti-HCV immunogenic peptide
RLGSGRNLV

SEQ ID NO: 293 - protein, 9 aa, Artificial Sequence
  anti-HCV immunogenic peptide
RLPSGHNLV

SEQ ID NO: 294 - protein, 9 aa, Artificial Sequence
  anti-HCV immunogenic peptide
RLPSGHNLA

SEQ ID NO: 295 - protein, 9 aa, Artificial Sequence
  anti-HCV immunogenic peptide
RLPSGRNLA

SEQ ID NO: 296 - protein, 9 aa, Artificial Sequence
  anti-HCV immunogenic peptide
RPPSGRNLV

SEQ ID NO: 297 - protein, 9 aa, Artificial Sequence
  anti-HCV immunogenic peptide
KLPSGRNLV

SEQ ID NO: 298 - protein, 9 aa, Artificial Sequence
  anti-HCV immunogenic peptide
RFPSGRKLV

SEQ ID NO: 299 - protein, 9 aa, Artificial Sequence
  anti-HCV immunogenic peptide
RLPSGRNPV

SEQ ID NO: 300 - protein, 9 aa, Artificial Sequence
  anti-HCV immunogenic peptide
RLPSGLNLV

SEQ ID NO: 301 - protein, 9 aa, Artificial Sequence
  anti-HCV immunogenic peptide
RLPSGRNIV

SEQ ID NO: 302 - protein, 9 aa, Artificial Sequence
  anti-HCV immunogenic peptide
RLPSGRSLV

SEQ ID NO: 303 - protein, 9 aa, Artificial Sequence
  anti-HCV immunogenic peptide
RLPSDRNLV

SEQ ID NO: 304 - protein, 9 aa, Artificial Sequence
  anti-HCV immunogenic peptide
RLPNGRNLV

SEQ ID NO: 305 - protein, 9 aa, Artificial Sequence
  anti-HCV immunogenic peptide
RLRSGRNLA

SEQ ID NO: 306 - protein, 9 aa, Artificial Sequence
  anti-HCV immunogenic peptide
RLRSGHNLV

SEQ ID NO: 307 - protein, 9 aa, Artificial Sequence
  anti-HCV immunogenic peptide
RLRSGRNLV

SEQ ID NO: 308 - protein, 9 aa, Artificial Sequence
  anti-HCV immunogenic peptide
RLQNGRNPV

SEQ ID NO: 309 - protein, 9 aa, Artificial Sequence
  anti-HCV immunogenic peptide
RLRNGRNPV

SEQ ID NO: 310 - protein, 9 aa, Artificial Sequence
  anti-HCV immunogenic peptide
RLRNGRNPA

SEQ ID NO: 311 - protein, 9 aa, Artificial Sequence
  anti-HCV immunogenic peptide
RIRNGRNPV

SEQ ID NO: 312 - protein, 9 aa, Artificial Sequence
  anti-HCV immunogenic peptide
RLQNGRNPA

SEQ ID NO: 313 - protein, 9 aa, Artificial Sequence
  anti-HCV immunogenic peptide
RLPSDHNLA

SEQ ID NO: 314 - protein, 9 aa, Artificial Sequence
  anti-HCV immunogenic peptide
RLPSGRSLA

SEQ ID NO: 315 - protein, 9 aa, Artificial Sequence
  anti-HCV immunogenic peptide
RLPSGHNPA

SEQ ID NO: 316 - protein, 9 aa, Artificial Sequence
  anti-HCV immunogenic peptide
KLPSGRNLA

SEQ ID NO: 317 - protein, 9 aa, Artificial Sequence
  anti-HCV immunogenic peptide
NLPSGRNLA

SEQ ID NO: 318 - protein, 9 aa, Artificial Sequence
  anti-HCV immunogenic peptide
KLLNGRSLA

SEQ ID NO: 319 - protein, 9 aa, Artificial Sequence
  anti-HCV immunogenic peptide
KLLNGHSLA

SEQ ID NO: 320 - protein, 9 aa, Artificial Sequence
  anti-HCV immunogenic peptide
KLLNGHSPA

SEQ ID NO: 321 - protein, 9 aa, Artificial Sequence
  anti-HCV immunogenic peptide
KLPSGQNPA

SEQ ID NO: 322 - protein, 9 aa, Artificial Sequence
  anti-HCV immunogenic peptide
RLQSGHNPA

SEQ ID NO: 323 - protein, 9 aa, Artificial Sequence
  anti-HCV immunogenic peptide
RLPSGHSLA

SEQ ID NO: 324 - protein, 9 aa, Artificial Sequence
  anti-HCV immunogenic peptide
RLLNGPSPE

SEQ ID NO: 325 - protein, 9 aa, Artificial Sequence
  anti-HCV immunogenic peptide
RLRNGPSPE

SEQ ID NO: 326 - protein, 9 aa, Artificial Sequence
  anti-HCV immunogenic peptide
RLQNDPSPE

SEQ ID NO: 327 - protein, 9 aa, Artificial Sequence
  anti-HCV immunogenic peptide
KLLNGPSPG

SEQ ID NO: 328 - protein, 9 aa, Artificial Sequence
  anti-HCV immunogenic peptide
KLLNGPSLG

SEQ ID NO: 329 - protein, 9 aa, Artificial Sequence
  anti-HCV immunogenic peptide
KLLNGPSPE

SEQ ID NO: 330 - protein, 9 aa, Artificial Sequence
  anti-HCV immunogenic peptide
RLPNGPSLE

SEQ ID NO: 331 - protein, 9 aa, Artificial Sequence
  anti-HCV immunogenic peptide
RPPNGPSLE

SEQ ID NO: 332 - protein, 9 aa, Artificial Sequence
  anti-HCV immunogenic peptide
RPQNGPSPE

SEQ ID NO: 333 - protein, 9 aa, Artificial Sequence
  anti-HCV immunogenic peptide
RLPNGPSPE

SEQ ID NO: 334 - protein, 9 aa, Artificial Sequence
  anti-HCV immunogenic peptide
RPPNGPNPE

SEQ ID NO: 335 - protein, 9 aa, Artificial Sequence
  anti-HCV immunogenic peptide
RPPSGPSPE

SEQ ID NO: 336 - protein, 9 aa, Artificial Sequence
  anti-HCV immunogenic peptide
RPLNDPSLE

SEQ ID NO: 337 - protein, 9 aa, Artificial Sequence
  anti-HCV immunogenic peptide
RLLNDPSPE

SEQ ID NO: 338 - protein, 9 aa, Artificial Sequence
  anti-HCV immunogenic peptide
RLPSDPSPE

SEQ ID NO: 339 - protein, 9 aa, Artificial Sequence
  anti-HCV immunogenic peptide
RLLSDPSPE

SEQ ID NO: 340 - protein, 9 aa, Artificial Sequence
  anti-HCV immunogenic peptide
RLPSDPSPG

SEQ ID NO: 341 - protein, 9 aa, Artificial Sequence
  anti-HCV immunogenic peptide
RLPSDPSLE

SEQ ID NO: 342 - protein, 9 aa, Artificial Sequence
  anti-HCV immunogenic peptide
RHPSDPSPE

SEQ ID NO: 343 - protein, 9 aa, Artificial Sequence
  anti-HCV immunogenic peptide
RLPSDPSRV

SEQ ID NO: 344 - protein, 9 aa, Artificial Sequence
  anti-HCV immunogenic peptide
SLPSDPSRV

SEQ ID NO: 345 - protein, 9 aa, Artificial Sequence
  anti-HCV immunogenic peptide
RLLSDPSRV

SEQ ID NO: 346 - protein, 9 aa, Artificial Sequence
  anti-HCV immunogenic peptide
RLLSDPSLV

SEQ ID NO: 347 - protein, 9 aa, Artificial Sequence
  anti-HCV immunogenic peptide
KPPNGPSHV

SEQ ID NO: 348 - protein, 9 aa, Artificial Sequence
  anti-HCV immunogenic peptide
KLPNGPSHV

SEQ ID NO: 349 - protein, 9 aa, Artificial Sequence
  anti-HCV immunogenic peptide
RLPNGPSHV

SEQ ID NO: 350 - protein, 9 aa, Artificial Sequence
  anti-HCV immunogenic peptide
KLPNDPSHV

SEQ ID NO: 351 - protein, 9 aa, Artificial Sequence
  anti-HCV immunogenic peptide
RLPNGPSHA

SEQ ID NO: 352 - protein, 9 aa, Artificial Sequence
  anti-HCV immunogenic peptide
KLPSGPSHV

SEQ ID NO: 353 - protein, 9 aa, Artificial Sequence
  anti-HCV immunogenic peptide
RLRSGPSHV

SEQ ID NO: 354 - protein, 9 aa, Artificial Sequence
  anti-HCV immunogenic peptide
ILRSGPSHV

SEQ ID NO: 355 - protein, 9 aa, Artificial Sequence
  anti-HCV immunogenic peptide
KLRSGPSHV

SEQ ID NO: 356 - protein, 9 aa, Artificial Sequence
  anti-HCV immunogenic peptide
RLRSDPSHV

SEQ ID NO: 357 - protein, 9 aa, Artificial Sequence
  anti-HCV immunogenic peptide
RLRSGPSLV

SEQ ID NO: 358 - protein, 9 aa, Artificial Sequence
  anti-HCV immunogenic peptide
RLPNGPSRV

SEQ ID NO: 359 - protein, 9 aa, Artificial Sequence
  anti-HCV immunogenic peptide
GPGLSPGTL

SEQ ID NO: 360 - protein, 9 aa, Artificial Sequence
  anti-HCV immunogenic peptide
SPGESQDIL

SEQ ID NO: 361 - protein, 9 aa, Artificial Sequence
  anti-HCV immunogenic peptide
SPGENRDIL

SEQ ID NO: 362 - protein, 9 aa, Artificial Sequence
  anti-HCV immunogenic peptide
SPGENQDIL

SEQ ID NO: 363 - protein, 9 aa, Artificial Sequence
  anti-HCV immunogenic peptide
SPGEGQDTL

SEQ ID NO: 364 - protein, 9 aa, Artificial Sequence
  anti-HCV immunogenic peptide
SPGEGQDIL

SEQ ID NO: 365 - protein, 9 aa, Artificial Sequence
  anti-HCV immunogenic peptide
SPGGDQDIL

SEQ ID NO: 366 - protein, 9 aa, Artificial Sequence
  anti-HCV immunogenic peptide
SPGEDQDIL

SEQ ID NO: 367 - protein, 9 aa, Artificial Sequence
  anti-HCV immunogenic peptide
SPGESQDTL

SEQ ID NO: 368 - protein, 9 aa, Artificial Sequence
  anti-HCV immunogenic peptide
SPGVGQDTL

SEQ ID NO: 369 - protein, 9 aa, Artificial Sequence
  anti-HCV immunogenic peptide
SPGEDQDTL

SEQ ID NO: 370 - protein, 9 aa, Artificial Sequence
  anti-HCV immunogenic peptide
SPGDVQDTP

SEQ ID NO: 371 - protein, 9 aa, Artificial Sequence
  anti-HCV immunogenic peptide
SPGDVQDTL

SEQ ID NO: 372 - protein, 9 aa, Artificial Sequence
  anti-HCV immunogenic peptide
SPGENQDTL

SEQ ID NO: 373 - protein, 9 aa, Artificial Sequence
  anti-HCV immunogenic peptide
SPGENQDTP

SEQ ID NO: 374 - protein, 9 aa, Artificial Sequence
  anti-HCV immunogenic peptide
SPGEHQDTL

SEQ ID NO: 375 - protein, 9 aa, Artificial Sequence
  anti-HCV immunogenic peptide
SPGESQDIP

SEQ ID NO: 376 - protein, 9 aa, Artificial Sequence
  anti-HCV immunogenic peptide
SPGGSQDTP

SEQ ID NO: 377 - protein, 9 aa, Artificial Sequence
  anti-HCV immunogenic peptide
RPGENQVAP

SEQ ID NO: 378 - protein, 9 aa, Artificial Sequence
  anti-HCV immunogenic peptide
SPGENQDIP

SEQ ID NO: 379 - protein, 9 aa, Artificial Sequence
  anti-HCV immunogenic peptide
NPGENQDTP

SEQ ID NO: 380 - protein, 9 aa, Artificial Sequence
  anti-HCV immunogenic peptide
SPGVNQDTP

SEQ ID NO: 381 - protein, 9 aa, Artificial Sequence
  anti-HCV immunogenic peptide
SPGDVQDIP

SEQ ID NO: 382 - protein, 9 aa, Artificial Sequence
  anti-HCV immunogenic peptide
SPGVAQDIP

SEQ ID NO: 383 - protein, 9 aa, Artificial Sequence
  anti-HCV immunogenic peptide
GTGLSPGIL

SEQ ID NO: 384 - protein, 9 aa, Artificial Sequence
  anti-HCV immunogenic peptide
GPGPSPGTL

SEQ ID NO: 385 - protein, 9 aa, Artificial Sequence
  anti-HCV immunogenic peptide
GPGLSPGIL

SEQ ID NO: 386 - protein, 9 aa, Artificial Sequence
  anti-HCV immunogenic peptide
GPGLSPGTP

SEQ ID NO: 387 - protein, 9 aa, Artificial Sequence
  anti-HCV immunogenic peptide
GPGLNPVTP

SEQ ID NO: 388 - protein, 9 aa, Artificial Sequence
  anti-HCV immunogenic peptide
GPGLSPGIP

SEQ ID NO: 389 - protein, 9 aa, Artificial Sequence
  anti-HCV immunogenic peptide
GPGLSRGIL

SEQ ID NO: 390 - protein, 9 aa, Artificial Sequence
  anti-HCV immunogenic peptide
GPGPSPGIL

SEQ ID NO: 391 - protein, 9 aa, Artificial Sequence
  anti-HCV immunogenic peptide
GPGLSLGIL

SEQ ID NO: 392 - protein, 9 aa, Artificial Sequence
  anti-HCV immunogenic peptide
GPGLSLGTL

SEQ ID NO: 393 - protein, 9 aa, Artificial Sequence
  anti-HCV immunogenic peptide
GPGRSPGTL

SEQ ID NO: 394 - protein, 9 aa, Artificial Sequence
  anti-HCV immunogenic peptide
EPGLSPGTL

SEQ ID NO: 395 - protein, 9 aa, Artificial Sequence
  anti-HCV immunogenic peptide
GPRLSPGTL

SEQ ID NO: 396 - protein, 9 aa, Artificial Sequence
  anti-HCV immunogenic peptide
GPGLSPGTH

SEQ ID NO: 397 - protein, 9 aa, Artificial Sequence
  anti-HCV immunogenic peptide
GPGLSLGTP

SEQ ID NO: 398 - protein, 9 aa, Artificial Sequence
  anti-HCV immunogenic peptide
GSGLSPGTP

SEQ ID NO: 399 - protein, 9 aa, Artificial Sequence
  anti-HCV immunogenic peptide
GPGHSPGTL

SEQ ID NO: 400 - protein, 9 aa, Artificial Sequence
  anti-HCV immunogenic peptide
GPGPSPGTP

SEQ ID NO: 401 - protein, 9 aa, Artificial Sequence
  anti-HCV immunogenic peptide
GPVLSPGTL

SEQ ID NO: 402 - protein, 9 aa, Artificial Sequence
  anti-HCV immunogenic peptide
GPGLSPDTL

SEQ ID NO: 403 - protein, 9 aa, Artificial Sequence
  anti-HCV immunogenic peptide
DPGRSPGIL

SEQ ID NO: 404 - protein, 9 aa, Artificial Sequence
  anti-HCV immunogenic peptide
GPGLSPGIH

SEQ ID NO: 405 - protein, 9 aa, Artificial Sequence
  anti-HCV immunogenic peptide
GPGLNPGTH

SEQ ID NO: 406 - protein, 9 aa, Artificial Sequence
  anti-HCV immunogenic peptide
GPGHNQDIH

SEQ ID NO: 407 - protein, 9 aa, Artificial Sequence
  anti-HCV immunogenic peptide
GPGHSQDTH

SEQ ID NO: 408 - protein, 9 aa, Artificial Sequence
  anti-HCV immunogenic peptide
DPGRSPGTL

SEQ ID NO: 409 - protein, 9 aa, Artificial Sequence
  anti-HCV immunogenic peptide
GPGVNPGTL

SEQ ID NO: 410 - protein, 9 aa, Artificial Sequence
  anti-HCV immunogenic peptide
VPGRSLGTL

SEQ ID NO: 411 - protein, 9 aa, Artificial Sequence
  anti-HCV immunogenic peptide
VPGRSLGTR

SEQ ID NO: 412 - protein, 9 aa, Artificial Sequence
  anti-HCV immunogenic peptide
VPGHSPGTL

SEQ ID NO: 413 - protein, 9 aa, Artificial Sequence
  anti-HCV immunogenic peptide
GPGPSLGTL

SEQ ID NO: 414 - protein, 9 aa, Artificial Sequence
  anti-HCV immunogenic peptide
DPGLSPGTL

SEQ ID NO: 415 - protein, 9 aa, Artificial Sequence
  anti-HCV immunogenic peptide
GPGRSLGTP

SEQ ID NO: 416 - protein, 9 aa, Artificial Sequence
  anti-HCV immunogenic peptide
VPGHSLGIP

SEQ ID NO: 417 - protein, 9 aa, Artificial Sequence
  anti-HCV immunogenic peptide
VPGRSPGTH

SEQ ID NO: 418 - protein, 9 aa, Artificial Sequence
  anti-HCV immunogenic peptide
VPGLSPGTH

SEQ ID NO: 419 - protein, 9 aa, Artificial Sequence
  anti-HCV immunogenic peptide
VPGLSLGTH

SEQ ID NO: 420 - protein, 9 aa, Artificial Sequence
  anti-HCV immunogenic peptide
VPGPSPGTP

SEQ ID NO: 421 - protein, 9 aa, Artificial Sequence
  anti-HCV immunogenic peptide
VPGHSLGTP

SEQ ID NO: 422 - protein, 9 aa, Artificial Sequence
  anti-HCV immunogenic peptide
VPGLSPGTP

SEQ ID NO: 423 - protein, 9 aa, Artificial Sequence
  anti-HCV immunogenic peptide
VPGLSPGTL

SEQ ID NO: 424 - protein, 9 aa, Artificial Sequence
  anti-HCV immunogenic peptide
VAGVNPATL

SEQ ID NO: 425 - protein, 9 aa, Artificial Sequence
  anti-HCV immunogenic peptide
VAGDNPATL

SEQ ID NO: 426 - protein, 9 aa, Artificial Sequence
  anti-HCV immunogenic peptide
VAGLSPATP

SEQ ID NO: 427 - protein, 9 aa, Artificial Sequence
  anti-HCV immunogenic peptide
GTGLSLVIP

SEQ ID NO: 428 - protein, 9 aa, Artificial Sequence
  anti-HCV immunogenic peptide
GTGRSLVTP

SEQ ID NO: 429 - protein, 9 aa, Artificial Sequence
  anti-HCV immunogenic peptide
GTGLSLVTL

SEQ ID NO: 430 - protein, 9 aa, Artificial Sequence
  anti-HCV immunogenic peptide
GTGLSGDTL

SEQ ID NO: 431 - protein, 9 aa, Artificial Sequence
  anti-HCV immunogenic peptide
GTGLSGDIL

SEQ ID NO: 432 - protein, 9 aa, Artificial Sequence
  anti-HCV immunogenic peptide
GPGVSLATP

SEQ ID NO: 433 - protein, 9 aa, Artificial Sequence
  anti-HCV immunogenic peptide
VPGVSLGTH

SEQ ID NO: 434 - protein, 9 aa, Artificial Sequence
  anti-HCV immunogenic peptide
VPGLNLGTH

SEQ ID NO: 435 - protein, 9 aa, Artificial Sequence
  anti-HCV immunogenic peptide
APDWVCARR

SEQ ID NO: 436 - protein, 9 aa, Artificial Sequence
  anti-HCV immunogenic peptide
KLLSGHSLA

(19) United States
(12) Patent Application Publication
(10) Pub. No.: US 2006/0269562 A1
(43) Pub. Date: Nov. 30, 2006

(54) POLYPEPTIDES F' OF THE HEPATITIS C VIRUS, T EPITOPEO, AND THE DIAGNOSTIC AND THERAPEUTIC APPLICATIONS THEREOF

(76) Inventors: Christine Bain, Soucieu en Jarrest (FR); Genevieve Inchauspe, Lyon (FR); Jean-Pierre Lavergne, Villeurbanne (FR); Peggy Parroche, Lyon (FR); Francois Penin, Decines (FR)

Correspondence Address: OLIFF & BERRIDGE, PLC, P.O. BOX 19928, ALEXANDRIA, VA, 22320, US

(21) Appl. No.: 10/541,353

(22) PCT Filed: Dec. 29, 2003
(86) PCT No.: PCT/FR03/03922

§ 371 Date: Jul. 5, 2005

(30) Foreign Application Priority Data
Jan. 7, 2003 (FR) 03/00094

(51) Int. Cl. C12Q 1/70; C07H 21/04; A61K 39/29; C12N 1/21; C12N 15/86; C07K 14/18

(52) U.S. Cl. 424/189.1; 530/350; 435/5; 435/69.3; 435/252.3; 435/456; 435/325; 536/23.72

(57) ABSTRACT

The present invention relates to novel polypeptides F′ from the protein F, said polypeptides inducing an immune response to the hepatitis C virus and consisting of 99 amino acids situated between positions 43 and 141 of the polyprotein of the hepatitis C virus, to four associated T epitopes consisting of 9 amino acids, and to the diagnostic and therapeutic applications thereof.

The present invention relates to a novel polypeptide of the hepatitis C virus, derived from a reading frame shift, that is useful in particular in prophylactic and therapeutic immunization directed against this virus.

Hepatitis C is the main cause of hepatitis acquired by transfusion. Hepatitis C can also be transmitted by other percutaneous routes, for example by intravenous drug injection. The risk of contamination among health professionals is not, moreover, insignificant.

Hepatitis C differs from the other forms of liver diseases associated with viruses, such as hepatitis A, B or D. Infections with the hepatitis C virus (HCV) are mainly chronic and result in liver diseases, such as hepatitis, cirrhosis and carcinoma in a large number of cases (5 to 20%).

Although the risk of virus transmission by transfusion has decreased due to the setting up of screening assays in the 1990s, the frequency of hepatitis C remains high. By way of example, a recent study indicates that there are, at this time, still thought to be 10 000 to 15 000 new cases of infection per year in France (S. Deuffic and al., Hepatology 1999; 29: 1596-1601). Currently, approximately 170 million individuals throughout the world are chronically infected with HCV. The high-risk populations are mainly hospital personnel and intravenous drug users, but there are asymptomatic blood donors who do not belong to these high-risk groups and in whom circulating anti-HCV antibodies have been found. For the latter, the route of infection has not yet been identified.

HCV was the first hepatotropic virus isolated by means of molecular biology techniques. The sequences of the viral genome were cloned before the viral particle were visualized.

HCV belongs to a new genus of the family Flaviviridae, the hepaciviruses. It is a positive, single-stranded RNA virus of 9.5 kb that replicates by means of a complementary RNA copy and the translation product of which is a polyprotein precursor of approximately 3 000 amino acids. The 5′ end of the HCV genome corresponds to an untranslated region adjacent to the genes that encode the structural proteins, the core protein of the nucleocapsid, the two envelope glycoproteins, E1 and E2, and a small protein called p7. The 5′ untranslated region and the core gene are relatively well conserved in the various genotypes. The E1 and E2 envelope proteins are encoded by regions that are more variable from one isolate to another. The p7 protein is an extremely hydrophobic protein that is thought to constitute an ion channel. The 3′ end of the HCV genome contains the genes that encode the non-structural proteins (NS2, NS3, NS4 and NS5) and a 3′ noncoding region that has a well conserved domain (Major M E, Feinstone S M, Hepatology, June 1997, 25(6): 1527-1538).

At the current time, the most effective therapy for treating hepatitis C combines pegylated interferon and ribavirin (Manns M P and al., The Lancet, Sep. 22, 2001, Vol. 358, 958-965). Although this therapy is particularly effective in the case of patients infected with viral strains belonging to genotypes 2 and 3, it still has only a limited effect on genotypes 1a, 1b and 4 (Manns M P, above). Less than 50% of patients treated become “long-term responders”.

It is therefore necessary to develop a vaccine composition that targets these “poor responder” genotypes as a priority.

Several studies today show that the control of an infection due to HCV, either naturally (“spontaneous resolution”) or after treatment (“therapeutic resolution”), is associated with the induction or the potentiation of cell-mediated immune responses involving CD4⁺ T and CD8⁺ T lymphocytes (Ward S., and al., 2002, Clin. Exp. Immunol., 128: 195-203).

The aim of vaccines based on the use of peptides is generally to induce immune responses mediated by CD4⁺ T lymphocytes and/or CD8⁺ T lymphocytes.

The molecules of the major histocompatibility complex (MHC or otherwise called HLA in humans) are referred to as class I or class II. The class I molecules are expressed on virtually all nucleated cells and are capable of presenting epitopes or peptides to CD8⁺ cytotoxic T lymphocytes (CTLs). The class II molecules are capable of presenting epitopes to CD4⁺ T cells, but their expression is restricted to antigen-presenting cells.

Patent application WO 99/63941 describes a novel protein of the hepatitis C virus, of 196 amino acids, that is not encoded by a conventional open reading frame of the HCV virus. In fact, this protein is an alternative protein to the core protein of the virus exhibiting a reading frame shift at position +1 or +2. That application also describes two peptides of 13 amino acids derived from this novel protein, which contain B epitopes and are therefore useful in particular for the production of antibodies for diagnosis. No cell-mediated response is shown in that document.

Z. Xu and al. (Xu, Z. et al., 2001, EMBO J., 20(14), 3840-3848), J. L. Walewski, and al. (2001, RNA, 7, 710-721) and also Varaklioti et al. (2001, J. Biol. Chem., 20(17), 17713-17721) confirm this open reading frame shift within the core protein. In these articles, the experiments described were carried ort essentially in vitro, with the exception of preliminary assays using sera from patients carrying HCV. These preliminary assays refer to the presence of antibodies specific for this novel alternative core protein, called protein F, in patients carrying HCV. These studies are focused on viral strains of genotype 1a.

None of these documents of the prior art either describes or suggests the existence of cell-mediated responses specific for this novel protein F in patients carrying HCV.

The applicant has demonstrated, unexpectedly, a novel polypeptide of 99 amino acids that induces a cell-mediated response in patients who are seropositive for HCV, and in particular the secretion of interleukin 10 (IL10), with or without production of gamma-interferon, and that is capable of inducing specific cellular immune responses in patients infected with viral strains, in particular of genotypes 1b and 3, whatever the HLA typing of the patient, but preferably for HLA-A2 and B7 patients.

As this polypeptide is a fragment of the protein F, it was called polypeptide F′.

Thus, a subject of the present invention is a polypeptide F′ that induces an immune response against the hepatitis C virus, characterized in that it consists of 99 amino acids located between positions 43 and 141 of the polyprotein of the hepatitis C virus.

Of course, the expression “located between positions 43 and 141 of the polyprotein of the hepatitis C virus” is intended to mean positions 43 to 141 of the polyprotein encoded with a shift of +1 on the reading frame, as previously indicated for the protein F. This nomenclature will subsequently be used for the positions with respect to the polyprotein of the virus.

A subject of the invention is also four particular T epitopes that are at least partially included in this protein F′, located at positions 40-48, 43-51, 50-58 and 73-81 of the HCV polyprotein, which epitopes induce an immune response, the nucleotide sequences encoding said proteins and said epitopes, the vectors that include these nucleotide sequences, and also the microorganisms or host cells cotransformed with these vectors.

Finally, a subject of the invention is also the antibodies directed against the polypeptides and epitopes of the invention, and also the use of the polypeptides, of the epitopes and of the antibodies, for preparing a drug intended to inhibit or prevent an infection caused by the hepatitis C virus, and also for preparing diagnostic compositions.

The polypeptides F′, and also the epitopes of the invention, are capable, unexpectedly, of inducing a cell-mediated response in patients who are seropositive for HCV, and in particular the secretion of interleukin 10 (IL10), with or without production of gamma-interferon.

The polypeptides F′ and epitopes were obtained from the Shimotono genotype 1b consensus sequence (EMBL D89872).

The polypeptides F′ have 99 amino acids, located at positions 43 and 141 of the hepatitis C virus poly-protein.

Throughout the remainder of the text, the term “polypeptide” or “epitope” will of course be intended to mean the polypeptides and epitopes having the natural amino acid sequences, originating from any strain and isolate of HCV, as defined in the sequence listing, and also the analogs, muteins and homologs thereof.

The epitopes are peptides that have approximately from 8 to 15 amino acids and the polypeptides are larger peptides, such that, in the definitions hereinafter, the term “peptide” will be used indifferently to denote an epitope or a polypeptide.

The terms “analogs” or “muteins” of a peptide is intended to mean the biologically active derivatives of the reference molecules that exhibit the desired activity, namely the ability to stimulate a cell-mediated immune response as defined above.

In general, the term “analog” refers to compounds that have a natural sequence and polypeptide structure exhibiting one or more amino acid additions, substitutions (generally conservative in terms of nature) and/or deletions relative to the natural molecule, provided that the modifications do not destroy the immunogenic activity. The term “mutein” is intended to mean the peptides having one or more elements that imitate the peptide (“peptoids”), such as those described in PCT patent application WO 91/04282. Preferably, the analog or the mutein has at least the same immunoactivity as the natural molecule. Methods for preparing polypeptide analogs and muteins are known to those skilled in the art and are described below.

The analogs that are particularly preferred include the substitutions that are conservative in nature, i.e. the substitutions that take place in a family of amino acids. Specifically, amino acids are generally divided up into 4 families, i.e. (1) acidic amino acids such as aspartate and glutamate, (2) basic amino acids such as lysine, arginine and histidine, (3) nonpolar amino acids such as alanine, leucine, isoleucine, proline, phenylalanine, methionine and tryptophan, and (4) uncharged polar amino acids such as glycine, asparagine, glutamine, cysteine, serine, threonine and tyrosine. Phenylalanine, tryptophan and tyrosine are sometimes classified as aromatic amino acids. For example, it can reasonably be predicted that the isolated replacement of leucine with isoleucine or valine, of an aspartate with a glutamate, or of a threonine with a serine, or a similar conservative replacement of an amino acid with another amino acid that is structurally related, will have no major effect on the biological activity. Those skilled in the art will readily determine the regions of the peptide molecule of interest that can tolerate a change with reference to the Hopp/Woods and Kyte-Doolite plots, that are well known in the art.

The term “homology” is intended to mean the percentage of identity between two peptide molecules. Two amino acid sequences are “substantially homologous” to one another when the sequences exhibit at least 60%, preferably at least 75%, more preferably at least 80-85%, more preferably at least 90%, and even more preferably at least 95-98% or more, sequence identity over a defined length of the peptide molecules.

In general, the term “identity” refers to an exact correspondence amino acid by amino acid of two peptide sequences. The percentage identity can be determined by direct comparison of the sequence information between two molecules by aligning the sequences, counting the exact number of mismatches between the two aligned sequences, dividing by the length of the shortest sequence, and multiplying the result by 100. The percentage identity can also be determined using computer programs such as ALIGN, M. O. Dayhoff, in Atlas of Protein Sequence and Structure M. O. Dayhoff ed., 1981, 5 Suppl., 3: 482-489.

According to a particular embodiment of the invention, the polypeptides F′ are derived from genotype 1b of HCV and have the sequence SEQ ID No.1 below: X₁WVCX₂X₃X₄X₅RLPSGX₆NX₇X₈X₉X₁₀X₁₁X₁₂LX₁₃X₁₄RX₁₅X₁₆X₁₇PRX₁₈GX₁₉GX₂₀SX₂₁GX₂₂X₂₃GX₂₄ SX₂₅X₂₆X₂₇RX₂₈X₂₉X₃₀GX₃₁DGSCX₃₂ PX₃₃X₃₄X₃₅GLX₃₆GAX₃₇X₃₈TPX₃₉X₄₀GX₄₁X₄₂X₄₃WVX₄₄SSX₄₅X₄₆X₄₇X₄₈X₄₉X₅₀X₅₁PX₅₂SWGX₅₃X₅₄RX₅₅SX₅₆, in which X₁ is G, D, E, V or S, X₂ is A or V, X₃ is R, H or Q, X₄ is L, R, P, S or G, X₅ is G or E, X₆ is R, L or H, X₇ is L or P, X₈ is V, E or A, X₉ is E, V, D or G, X₁₀ is G or D, X₁₁ is D or V, X₁₂ is N or S, X₁₃ is S or F, X₁₄ is P or Q, X₁₅ is L, H, R, F, P or C, X₁₆ is A, V or I, X₁₇ is G, S, D, N, I or V, X₁₈ is A, V or E, X₁₉ is P, S or T, X₂₀ is L, P, H or R, X₂₁ is P or L, X₂₂ is T or I, X₂₃ is L, P or H, X₂₄ is P or L, X₂₅ is M or T, X₂₆ is A, V or P, X₂₇ is M, I or T, X₂₈ is A or V, X₂₉ is W, A, L or V, X₃₀ is G or D, X₃₁ is Q, L or R, X₃₂ is H, L, P or R, X₃₃ is V, A, E, K or T, X₃₄ is A or V, X₃₅ is L, R, H or P, X₃₆ is V, A, I or G, X₃₇ is P or L, X₃₈ is R, Q, L, M, T, E or P, X₃₉ is G or D, X₄₀ is V, A or G, X₄₁ is R or H, X₄₂ is V or A, X₄₃ is I or T, X₄₄ is R, G or K, X₄₅ is I or T, X₄₆ is P or L, X₄₇ is S or L, X₄₈ is H or R, X₄₉ is A or V, X₅₀ is A, V or G, X₅₁ is S or L, X₅₂ is T or I, X₅₃ is T or I, X₅₄ is F, Y or S, X₅₅ is S or L and X₅₆ is A, V, G or H.

Preferably, the polypeptide F′ is chosen from the following polypeptides:

the polypeptide of sequence SEQ ID No.2 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is V, X₃₄ is A, X₃₅ is L, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.3 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is V, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is V, X₃₅ is L, X₃₆ is V, X₃₇ is P, X₃₈ is Q, X₃₉ is G, X₄₀ is V, X₄₁ is R, X₄₂ is V, X₄₃ is I, X₄₄ is K, X₄₅ is I, X₄₆ is P, X₄₇ is S, X₄₈ is H, X₄₉ is A, X₅₀ is A, X₅₁ is S, X₅₂ is T, X₅₃ is T, X₅₄ is S, X₅₅ is S and X₅₆ is A,

the polypeptide of sequence SEQ ID No.4 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is V, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is L, X₃₆ is V, X₃₇ is P, X₃₈ is Q, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is S, X₅₅ is S and X₅₆ is A,

the polypeptide of sequence SEQ ID No.5 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is V, X₁₉ is P, X₂₀ is L, X₂₁ is P, X₂₂ is T, X₂₃ is L, X₂₄ is P, X₂₅ is L, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is L, X₃₆ is V, X₃₇ is P, X₃₈ is Q, X₃₉ is G, X₄₀ is V, X₄₁ is R, X₄₂ is V, X₄₃ is T, X₄₄ is K, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.6 which corresponds to the sequence SEQ ID No.1 in which X₁ is G, X₂ is V, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is L, X₂₂ is I, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is L, X₃₆ is V, X₃₇ is L, X₃₈ is M, X₃₉ is G, X₄₀ is V, X₄₁ is R, X₄₂ is V, X₄₃ is I, X₄₄ is R, X₄₅ is I, X₄₆ is P, X₄₇ is L, X₄₈ is H, X₄₉ is A, X₅₀ is A, X₅₁ is S, X₅₂ is T, X₅₃ is T, X₅₄ is S, X₅₅ is L and X₅₆ is A,

the polypeptide of sequence SEQ ID No.7 which corresponds to the sequence SEQ ID No.1 in which X₁ is G, X₂ is V, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is L, X₂₂ is I, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is L, X₃₆ is V, X₃₇ is P, X₃₈ is M, X₃₉ is G, X₄₀ is V, X₄₁ is R, X₄₂ is V, X₄₃ is I, X₄₄ is R, X₄₅ is I, X₄₆ is P, X₄₇ is L, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.8 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is I, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is L, X₃₆ is A, X₃₇ is L, X₃₈ is P, X₃₉ is G, X₄₀ is V, X₄₁ is R, X₄₂ is V, X₄₃ is T, X₄₄ is R, X₄₅ is I, X₄₆ is P, X₄₇ is L, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.9 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is L, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is L, X₃₆ is V, X₃₇ is P, X₃₈ is Q, X₃₉ is G, X₄₀ is V, X₄₁ is R, X₄₂ is V, X₄₃ is I, X₄₄ is R, X₄₅ is I, X₄₆ is P, X₄₇ is L, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.10 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is L, X₃₆ is V, X₃₇ is P, X₃₈ is L, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.11 which corresponds to the sequence SEQ ID No.1 in which X₁ is G, X₂ is A, X₃ is H, X₄ is L, X₅ is G, X₆ is R, X₇ is L, X₈ is V, X₉ is E, X₁₀ is G, X₁₁ is D, X₁₂ is N, X₁₃ is F, X₁₄ is P, X₁₅ is L, X₁₆ is V, X₁₇ is G, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is E, X₃₄ is A, X₃₅ is L, X₃₆ is V, X₃₇ is P, X₃₈ is L, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.12 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is L, X₃₆ is V, X₃₇ is P, X₃₈ is T, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.13 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is L, X₃₆ is V, X₃₇ is P, X₃₈ is M, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.14 which corresponds to the sequence SEQ ID No.1 in which X₁ is G, X₂ is A, X₃ is R, X₄ is L, X₅ is G, X₆ is R, X₇ is L, X₈ is A, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is L, X₃₆ is V, X₃₇ is P, X₃₈ is R, X₃₉ is G, X₄₀ is V, X₄₁ is R, X₄₂ is A, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.15 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is N, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is L, X₃₆ is V, X₃₇ is P, X₃₈ is R, X₃₉ is G, X₄₀ is V, X₄₁ is R, X₄₂ is A, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.16 which corresponds to the sequence SEQ ID No.1 in which X₁ is G, X₂ is A, X₃ is R, X₄ is L, X₅ is G, X₆ is H, X₇ is L, X₈ is A, X₉ is E, X₁₀ is G, X₁₁ is D, X₁₂ is N, X₁₃ is S, X₁₄ is P, X₁₅ is L, X₁₆ is A, X₁₇ is I, X₁₈ is A, X₁₉ is P, X₂₀ is L, X₂₁ is L, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is L, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is L, X₅₂ is T, X₅₃ is T, X₅₄ is F, X₅₅ is S and X₅₆ is A,

the polypeptide of sequence SEQ ID No.17 which corresponds to the sequence SEQ ID No.1 in which X₁ is G, X₂ is A, X₃ is R, X₄ is L, X₅ is G, X₆ is H, X₇ is L, X₈ is A, X₉ is E, X₁₀ is G, X₁₁ is D, X₁₂ is N, X₁₃ is S, X₁₄ is P, X₁₅ is L, X₁₆ is A, X₁₇ is I, X₁₈ is A, X₁₉ is P, X₂₀ is L, X₂₁ is L, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is R, X₃₃ is A, X₃₄ is A, X₃₅ is L, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is L, X₅₂ is T, X₅₃ is T, X₅₄ is F, X₅₅ is S and X₅₆ is A,

the polypeptide of sequence SEQ ID No.18 which corresponds to the sequence SEQ ID No.1 in which X₁ is V, X₂ is A, X₃ is R, X₄ is L, X₅ is G, X6 is H, X₇ is L, X₈ is V, X₉ is E, X₁₀ is G, X₁₁ is D, X₁₂ is N, X₁₃ is F, X₁₄ is P, X₁₅ is L, X₁₆ is A, X₁₇ is G, X₁₈ is A, X₁₉ is P, X₂₀ is L, X₂₁ is L, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is L, X₃₆ is V, X₃₇ is P, X₃₈ is L, X₃₉ is G, X₄₀ is V, X₄₁ is R, X₄₂ is A, X₄₃ is I, X₄₄ is R, X₄₅ is I, X₄₆ is P, X₄₇ is S, X₄₈ is H, X₄₉ is A, X₅₀ is A, X₅₁ is S, X₅₂ is I, X₅₃ is T, X₅₄ is F, X₅₅ is S and X₅₆ is A,

the polypeptide of sequence SEQ ID No.19 which corresponds to the sequence SEQ ID No.1 in which X₁ is G, X₂ is A, X₃ is R, X₄ is L, X₅ is G, X₆ is H, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is P, X₂₅ is T, X₂₆ is A, X₂₇ is M, X₂₈ is A, X₂₉ is W, X₃₀ is G, X₃₁ is Q, X₃₂ is H, X₃₃ is V, X₃₄ is A, X₃₅ is L, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.20 which corresponds to the sequence SEQ ID No.1 in which X₁ is G, X₂ is A, X₃ is R, X₄ is L, X₅ is G, X₆ is R, X₇ is L, X8 is V, X₉ is E, X₁₀ is G, X₁₁ is D, X₁₂ is N, X₁₃ is S, X₁₄ is P, X₁₅ is F, X₁₆ is A, X₁₇ is G, X₁₈ is A, X₁₉ is P, X₂₀ is L, X₂₁ is P, X₂₂ is T, X₂₃ is L, X₂₄ is P, X₂₅ is T, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is L, X₃₆ is V, X₃₇ is P, X₃₈ is R, X₃₉ is G, X₄₀ is V, X₄₁ is R, X₄₂ is V, X₄₃ is T, X₄₄ is R, X₄₅ is I, X₄₆ is P, X₄₇ is S, X₄₈ is H, X₄₉ is A, X₅₀ is A, X₅₁ is S, X₅₂ is I, X₅₃ is T, X₅₄ is F, X₅₅ is S and X₅₆ is A,

the polypeptide of sequence SEQ ID No.21 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is P, X₂₅ is T, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is L, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.22 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is F, X₁₆ is A, X₁₇ is D, X₁₈ is A, X₁₉ is P, X₂₀ is L, X₂₁ is P, X₂₂ is T, X₂₃ is L, X₂₄ is P, X₂₅ is M, X₂₆ is P, X₂₇ is T, X₂₈ is V, X₂₉ is W, X₃₀ is G, X₃₁ is Q, X₃₂ is H, X₃₃ is V, X₃₄ is A, X₃₅ is L, X₃₆ is V, X₃₇ is P, X₃₈ is R, X₃₉ is G, X₄₀ is V, X₄₁ is R, X₄₂ is A, X₄₃ is I, X₄₄ is R, X₄₅ is I, X₄₆ is P, X₄₇ is S, X₄₈ is R, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.23 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is D, X₁₈ is A, X₁₉ is P, X₂₀ is L, X₂₁ is P, X₂₂ is T, X₂₃ is L, X₂₄ is P, X₂₅ is M, X₂₆ is P, X₂₇ is T, X₂₈ is V, X₂₉ is W, X₃₀ is G, X₃₁ is Q, X₃₂ is H, X₃₃ is E, X₃₄ is A, X₃₅ is P, X₃₆ is V, X₃₇ is P, X₃₈ is R, X₃₉ is G, X₄₀ is V, X₄₁ is R, X₄₂ is A, X₄₃ is I, X₄₄ is R, X₄₅ is I, X₄₆ is P, X₄₇ is L, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.24 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is P, X₂₅ is M, X₂₆ is V, X₂₇ is T, X₂₈ is V, X₂₉ is W, X₃₀ is G, X₃₁ is Q, X₃₂ is H, X₃₃ is E, X₃₄ is A, X₃₅ is L, X₃₆ is A, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is L, X₄₈ is R, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.25 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is D, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is V, X₂₉ is W, X₃₀ is G, X₃₁ is Q, X₃₂ is H, X₃₃ is E, X₃₄ is A, X₃₅ is L, X₃₆ is V, X₃₇ is P, X₃₈ is R, X₃₉ is G, X₄₀ is V, X₄₁ is R, X₄₂ is A, X₄₃ is I, X₄₄ is R, X₄₅ is I, X₄₆ is P, X₄₇ is L, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.26 which corresponds to the sequence SEQ ID No.1 in which X₁ is G, X₂ is A, X₃ is R, X₄ is L, X₅ is G, X6 is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is V, X₂₉ is W, X₃₀ is G, X₃₁ is Q, X₃₂ is H, X₃₃ is G, X₃₄ is A, X₃₅ is L, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is L, X₄₈ is H, X₄₉ is A, X₅₀ is A, X₅₁ is S, X₅₂ is T, X₅₃ is T, X₅₄ is F, X₅₅ is L and X₅₆ is A,

the polypeptide of sequence SEQ ID No.27 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is P, X₂₅ is T, X₂₆ is A, X₂₇ is M, X₂₈ is V, X₂₉ is W, X₃₀ is G, X₃₁ is Q, X₃₂ is H, X₃₃ is E, X₃₄ is A, X₃₅ is L, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is L, X₄₈ is H, X₄₉ is A, X₅₀ is A, X₅₁ is S, X₅₂ is T, X₅₃ is T, X₅₄ is F, X₅₅ is L and X₅₆ is A,

the polypeptide of sequence SEQ ID No.28 which corresponds to the sequence SEQ ID No.1 in which X₁ is S, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is P, X₂₁ is P, X₂₂ is T, X₂₃ is P, X₂₄ is P, X₂₅ is M, X₂₆ is A, X₂₇ is M, X₂₈ is V, X₂₉ is W, X₃₀ is G, X₃₁ is Q, X₃₂ is H, X₃₃ is E, X₃₄ is A, X₃₅ is L, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is L, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.29 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is F, X₁₆ is A, X₁₇ is G, X₁₈ is A, X₁₉ is P, X₂₀ is L, X₂₁ is P, X₂₂ is T, X₂₃ is L, X₂₄ is P, X₂₅ is M, X₂₆ is A, X₂₇ is T, X₂₈ is V, X₂₉ is W, X₃₀ is G, X₃₁ is Q, X₃₂ is H, X₃₃ is V, X₃₄ is A, X₃₅ is L, X₃₆ is I, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is L, X₄₈ is H, X₄₉ is A, X₅₀ is G, X₅₁ is S, X₅₂ is T, X₅₃ is T, X₅₄ is F, X₅₅ is S and X₅₆ is A,

the polypeptide of sequence SEQ ID No.30 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is F, X₁₆ is A, X₁₇ is G, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is V, X₂₉ is W, X₃₀ is G, X₃₁ is Q, X₃₂ is H, X₃₃ is G, X₃₄ is A, X₃₅ is L, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is L, X₄₈ is H, X₄₉ is A, X₅₀ is G, X₅₁ is S, X₅₂ is T, X₅₃ is T, X₅₄ is F, X₅₅ is S and X₅₆ is A,

the polypeptide of sequence SEQ ID No.31 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is V, X₁₉ is P, X₂₀ is L, X₂₁ is P, X₂₂ is T, X₂₃ is L, X₂₄ is P, X₂₅ is M, X₂₆ is A, X₂₇ is T, X₂₈ is V, X₂₉ is W, X₃₀ is G, X₃₁ is Q, X₃₂ is H, X₃₃ is V, X₃₄ is A, X₃₅ is L, X₃₆ is V, X₃₇ is P, X₃₈ is Q, X₃₉ is G, X₄₀ is V, X₄₁ is R, X₄₂ is V, X₄₃ is I, X₄₄ is R, X₄₅ is I, X₄₆ is P, X₄₇ is L, X₄₈ is H, X₄₉ is A, X₅₀ is A, X₅₁ is S, X₅₂ is T, X₅₃ is T, X₅₄ is F, X₅₅ is L and X₅₆ is A,

the polypeptide of sequence SEQ ID No.32 which corresponds to the sequence SEQ ID No.1 in which X₁ is G, X₂ is A, X₃ is R, X₄ is L, X₅ is G, X₆ is R, X₇ is I, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is V, X₁₉ is P, X₂₀ is L, X₂₁ is P, X₂₂ is T, X₂₃ is L, X₂₄ is P, X₂₅ is M, X₂₆ is A, X₂₇ is T, X₂₈ is V, X₂₉ is W, X₃₀ is G, X₃₁ is Q, X₃₂ is H, X₃₃ is V, X₃₄ is A, X₃₅ is L, X₃₆ is V, X₃₇ is P, X₃₈ is Q, X₃₉ is G, X₄₀ is V, X₄₁ is R, X₄₂ is V, X₄₃ is I, X₄₄ is R, X₄₅ is I, X₄₆ is P, X₄₇ is L, X₄₈ is H, X₄₉ is A, X₅₀ is A, X₅₁ is S, X₅₂ is T, X₅₃ is T, X₅₄ is F, X₅₅ is L and X₅₆ is A,

the polypeptide of sequence SEQ ID No.33 which corresponds to the sequence SEQ ID No.1 in which X₁ is G, X₂ is A, X₃ is R, X₄ is L, X₅ is G, X6 is R, X₇ is L, X₈ is V, X₉ is E, X₁₀ is G, X₁₁ is H, X₁₂ is N, X₁₃ is S, X₁₄ is P, X₁₅ is L, X₁₆ is A, X₁₇ is G, X₁₈ is V, X₁₉ is P, X₂₀ is L, X₂₁ is P, X₂₂ is T, X₂₃ is L, X₂₄ is P, X₂₅ is M, X₂₆ is A, X₂₇ is T, X₂₈ is V, X₂₉ is W, X₃₀ is G, X₃₁ is Q, X₃₂ is H, X₃₃ is V, X₃₄ is A, X₃₅ is R, X₃₆ is V, X₃₇ is P, X₃₈ is R, X₃₉ is G, X₄₀ is V, X₄₁ is R, X₄₂ is A, X₄₃ is I, X₄₄ is R, X₄₅ is I, X₄₆ is P, X₄₇ is L, X₄₈ is H, X₄₉ is A, X₅₀ is A, X₅₁ is S, X₅₂ is T, X₅₃ is T, X₅₄ is F, X₅₅ is L and X₅₆ is A,

the polypeptide of sequence SEQ ID No.34 which corresponds to the sequence SEQ ID No.1 in which X₁ is G, X₂ is A, X₃ is R, X₄ is G, X₅ is G, X₆ is R, X₇ is L, X₈ is V, X₉ is E, X₁₀ is A, X₁₁ is D, X₁₂ is N, X₁₃ is S, X₁₄ is P, X₁₅ is L, X₁₆ is A, X₁₇ is V, X₁₈ is E, X₁₉ is P, X₂₀ is L, X₂₁ is P, X₂₂ is T, X₂₃ is L, X₂₄ is P, X₂₅ is M, X₂₆ is A, X₂₇ is T, X₂₈ is V, X₂₉ is W, X₃₀ is G, X₃₁ is L, X₃₂ is H, X₃₃ is E, X₃₄ is A, X₃₅ is P, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.35 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is P, X₂₅ is M, X₂₆ is A, X₂₇ is T, X₂₈ is V, X₂₉ is W, X₃₀ is G, X₃₁ is Q, X₃₂ is H, X₃₃ is E, X₃₄ is A, X₃₅ is P, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.36 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is V, X₂₉ is W, X₃₀ is G, X₃₁ is Q, X₃₂ is H, X₃₃ is E, X₃₄ is A, X₃₅ is L, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is Q, X₄₉ is V, X₅₀ is A, X₅₁ is S, X₅₂ is T, X₅₃ is T, X₅₄ is F, X₅₅ is S and X₅₆ is A,

the polypeptide of sequence SEQ ID No.37 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is P, X₂₄ is P, X₂₅ is M, X₂₆ is A, X₂₇ is M, X₂₈ is V, X₂₉ is W, X₃₀ is G, X₃₁ is Q, X₃₂ is H, X₃₃ is A, X₃₄ is A, X₃₅ is L, X₃₆ is V, X₃₇ is P, X₃₈ is Q, X₃₉ is G, X₄₀ is A, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.38 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is P, X₂₄ is P, X₂₅ is M, X₂₆ is A, X₂₇ is M, X₂₈ is V, X₂₉ is W, X₃₀ is G, X₃₁ is Q, X₃₂ is H, X₃₃ is A, X₃₄ is A, X₃₅ is L, X₃₆ is V, X₃₇ is P, X₃₈ is Q, X₃₉ is G, X₄₀ is A, X₄₁ is R, X₄₂ is V, X₄₃ is I, X₄₄ is G, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.39 which corresponds to the sequence SEQ ID No.1 in which X₁ is G, X₂ is A, X₃ is R, X₄ is L, X₅ is G, X₆ is R, X₇ is L, X₈ is V, X₉ is E, X₁o is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is P, X₂₄ is P, X₂₅ is M, X₂₆ is A, X₂₇ is M, X₂₈ is V, X₂₉ is W, X₃₀ is G, X₃₁ is Q, X₃₂ is H, X₃₃ is V, X₃₄ is A, X₃₅ is L, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.40 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is L, X₂₅ is M, X₂₆ is A, X₂₇ is M, X₂₈ is A, X₂₉ is W, X₃₀ is G, X₃₁ is Q, X₃₂ is H, X₃₃ is V, X₃₄ is A, X₃₅ is L, X₃₆ is V, X₃₇ is P, X₃₈ is R, X₃₉ is G, X₄₀ is V, X₄₁ is R, X₄₂ is V, X₄₃ is T, X₄₄ is R, X₄₅ is I, X₄₆ is P, X₄₇ is L, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.41 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is L, X₂₅ is M, X₂₆ is A, X₂₇ is M, X₂₈ is V, X₂₉ is W, X₃₀ is G, X₃₁ is Q, X₃₂ is R, X₃₃ is V, X₃₄ is A, X₃₅ is L, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.42 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is L, X₂₅ is M, X₂₆ is A, X₂₇ is M, X₂₈ is V, X₂₉ is W, X₃₀ is G, X₃₁ is Q, X₃₂ is R, X₃₃ is V, X₃₄ is A, X₃₅ is P, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.43 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is V, X₃₄ is A, X₃₅ is L, X₃₆ is V, X₃₇ is P, X₃₈ is Q, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is L and X₅₆ is A,

the polypeptide of sequence SEQ ID No.44 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is V, X₃₄ is A, X₃₅ is L, X₃₆ is V, X₃₇ is P, X₃₈ is Q, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.45 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is D, X₁₈ is A, X₁₉ is P, X₂₀ is L, X₂₁ is P, X₂₂ is I, X₂₃ is L, X₂₄ is P, X₂₅ is M, X₂₆ is A, X₂₇ is M, X₂₈ is A, X₂₉ is L, X₃₀ is G, X₃₁ is Q, X₃₂ is H, X₃₃ is A, X₃₄ is A, X₃₅ is P, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.46 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is D, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is P, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is I, X₅₄ is F, X₅₅ is S and X₅₆ is A,

the polypeptide of sequence SEQ ID No.47 which corresponds to the sequence SEQ ID No.1 in which X₁ is G, X₂ is A, X₃ is R, X₄ is L, X₅ is G, X₆ is R, X₇ is L, X₈ is V, X₉ is D, X₁₀ is G, X₁₁ is D, X₁₂ is N, X₁₃ is S, X₁₄ is P, X₁₅ is L, X₁₆ is A, X₁₇ is D, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is P, X₃₆ is V, X₃₇ is P, X₃₈ is R, X₃₉ is G, X₄₀ is V, X₄₁ is R, X₄₂ is V, X₄₃ is T, X₄₄ is R, X₄₅ is I, X₄₆ is P, X₄₇ is L, X₄₈ is H, X₄₉ is A, X₅₀ is A, X₅₁ is S, X₅₂ is I, X₅₃ is I, X₅₄ is F, X₅₅ is S and X₅₆ is A,

the polypeptide of sequence SEQ ID No.48 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is Q, X₁₅ is L, X₁₆ is A, X₁₇ is D, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is P, X₃₆ is V, X₃₇ is P, X₃₈ is R, X₃₉ is G, X₄₀ is V, X₄₁ is R, X₄₂ is V, X₄₃ is T, X₄₄ is R, X₄₅ is I, X₄₆ is P, X₄₇ is L, X₄₈ is H, X₄₉ is A, X₅₀ is A, X₅₁ is S, X₅₂ is I, X₅₃ is T, X₅₄ is F, X₅₅ is S and X₅₆ is A,

the polypeptide of sequence SEQ ID No.49 which corresponds to the sequence SEQ ID No.1 in which X₁ is G, X₂ is V, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is Q, X₁₅ is L, X₁₆ is A, X₁₇ is D, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is P, X₃₆ is V, X₃₇ is P, X₃₈ is R, X₃₉ is G, X₄₀ is V, X₄₁ is R, X₄₂ is V, X₄₃ is T, X₄₄ is R, X₄₅ is I, X₄₆ is P, X₄₇ is L, X₄₈ is H, X₄₉ is A, X₅₀ is A, X₅₁ is S, X₅₂ is I, X₅₃ is T, X₅₄ is F, X₅₅ is S and X₅₆ is A,

the polypeptide of sequence SEQ ID No.50 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is N, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is V, X₃₄ is A, X₃₅ is P, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is L, X₄₈ is H, X₄₉ is A, X₅₀ is A, X₅₁ is S, X₅₂ is I, X₅₃ is T, X₅₄ is F, X₅₅ is S and X₅₆ is A,

the polypeptide of sequence SEQ ID No.51 which corresponds to the sequence SEQ ID No.1 in which X₁ is G, X₂ is A, X₃ is R, X₄ is L, X₅ is G, X6 is R, X₇ is L, X₈ is V, X₉ is E, X₁₀ is G, X₁₁ is D, X₁₂ is N, X₁₃ is S, X₁₄ is P, X₁₅ is L, X₁₆ is V, X₁₇ is D, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is P, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is L, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.52 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is N, X₁₈ is V, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is P, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is I, X₅₃ is T, X₅₄ is F, X₅₅ is S and X₅₆ is A,

the polypeptide of sequence SEQ ID No.53 which corresponds to the sequence SEQ ID No.1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is N, X₁₈ is V, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is P, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.54 which corresponds to the sequence SEQ ID No.1 in which X₁ is G, X₂ is A, X₃ is R, X₄ is P, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is L, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is P, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is I, X₅₃ is T, X₅₄ is F, X₅₅ is S and X₅₆ is A,

the polypeptide of sequence SEQ ID No.55 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is P, X₃₆ is V, X₃₇ is P, X₃₈ is R, X₃₉ is G, X₄₀ is V, X₄₁ is R, X₄₂ is V, X₄₃ is T, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.56 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is S, X₁₃ is S, X₁₄ is P, X₁₅ is L, X₁₆ is A, X₁₇ is G, X₁₈ is V, X₁₉ is P, X₂₀ is L, X₂₁ is P, X₂₂ is T, X₂₃ is L, X₂₄ is P, X₂₅ is M, X₂₆ is A, X₂₇ is M, X₂₈ is V, X₂₉ is W, X₃₀ is G, X₃₁ is Q, X₃₂ is H, X₃₃ is V, X₃₄ is A, X₃₅ is P, X₃₆ is V, X₃₇ is P, X₃₈ is P, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.57 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is S, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is V, X₂₉ is W, X₃₀ is G, X₃₁ is Q, X₃₂ is H, X₃₃ is K, X₃₄ is A, X₃₅ is L, X₃₆ is V, X₃₇ is P, X₃₈ is E, X₃₉ is G, X₄₀ is V, X₄₁ is R, X₄₂ is V, X₄₃ is T, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.58 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is S, X₁₃ is S, X₁₄ is P, X₁₅ is L, X₁₆ is A, X₁₇ is S, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is V, X₃₄ is A, X₃₅ is P, X₃₆ is V, X₃₇ is P, X₃₈ is Q, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.59 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is S, X₁₃ is S, X₁₄ is P, X₁₅ is L, X₁₆ is A, X₁₇ is G, X₁₈ is A, X₁₉ is P, X₂₀ is H, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is V, X₃₄ is A, X₃₅ is P, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No.60 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is S, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is P, X₃₆ is V, X₃₇ is P, X₃₈ is R, X₃₉ is G, X₄₀ is V, X₄₁ is R, X₄₂ is V, X₄₃ is I, X₄₄ is R, X₄₅ is T, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No61 which corresponds to the sequence SEQ ID No1 in which X₁ is G, X₂ is A, X₃ is R, X₄ is S, X₅ is G, X₆ is R, X₇ is P, X₈ is V, X₉ is E, X₁₀ is G, X₁₁ is D, X₁₂ is S, X₁₃ is S, X₁₄ is P, X₁₅ is L, X₁₆ is A, X₁₇ is G, X₁₈ is A, X₁₉ is P, X₂₀ is L, X₂₁ is P, X₂₂ is I, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is P, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No62 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is S, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is L, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No63 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is I, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is R, X₃₃ is V, X₃₄ is A, X₃₅ is P, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No64 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is I, X₂₃ is L, X₂₄ is P, X₂₅ is T, X₂₆ is A, X₂₇ is T, X₂₈ is A, X₂₉ is W, X₃₀ is G, X₃₁ is Q, X₃₂ is H, X₃₃ is V, X₃₄ is V, X₃₅ is P, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No65 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is R, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is V, X₃₄ is A, X₃₅ is P, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is I, X₅₄ is F, X₅₅ is S and X₅₆ is A,

the polypeptide of sequence SEQ ID No66 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is S, X₁₃ is S, X₁₄ is P, X₁₅ is L, X₁₆ is A, X₁₇ is N, X₁₈ is A, X₁₉ is P, X₂₀ is L, X₂₁ is P, X₂₂ is T, X₂₃ is L, X₂₄ is P, X₂₅ is M, X₂₆ is A, X₂₇ is T, X₂₈ is A, X₂₉ is W, X₃₀ is G, X₃₁ is Q, X₃₂ is L, X₃₃ is V, X₃₄ is A, X₃₅ is P, X₃₆ is V, X₃₇ is P, X₃₈ is Q, X₃₉ is G, X₄₀ is V, X₄₁ is R, X₄₂ is V, X₄₃ is I, X₄₄ is R, X₄₅ is T, X₄₆ is P, X₄₇ is S, X₄₈ is H, X₄₉ is A, X₅₀ is A, X₅₁ is S, X₅₂ is T, X₅₃ is T, X₅₄ is F, X₅₅ is L and X₅₆ is A,

the polypeptide of sequence SEQ ID No67 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is S, X₁₃ is S, X₁₄ is P, X₁₅ is L, X₁₆ is A, X₁₇ is N, X₁₈ is A, X₁₉ is P, X₂₀ is L, X₂₁ is P, X₂₂ is T, X₂₃ is L, X₂₄ is P, X₂₅ is M, X₂₆ is A, X₂₇ is T, X₂₈ is V, X₂₉ is W, X₃₀ is G, X₃₁ is Q, X₃₂ is L, X₃₃ is V, X₃₄ is A, X₃₅ is P, X₃₆ is V, X₃₇ is P, X₃₈ is Q, X₃₉ is G, X₄₀ is V, X₄₁ is R, X₄₂ is V, X₄₃ is I, X₄₄ is R, X₄₅ is T, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No68 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is P, X₂₅ is M, X₂₆ is A, X₂₇ is T, X₂₈ is V, X₂₉ is W, X₃₀ is G, X₃₁ is Q, X₃₂ is H, X₃₃ is V, X₃₄ is A, X₃₅ is P, X₃₆ is V, X₃₇ is P, X₃₈ is Q, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is S, X₅₅ is S and X₅₆ is A,

the polypeptide of sequence SEQ ID No69 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is F, X₁₆ is A, X₁₇ is S, X₁₈ is A, X₁₉ is P, X₂₀ is L, X₂₁ is P, X₂₂ is T, X₂₃ is L, X₂₄ is P, X₂₅ is M, X₂₆ is A, X₂₇ is T, X₂₈ is A, X₂₉ is W, X₃₀ is G, X₃₁ is Q, X₃₂ is H, X₃₃ is V, X₃₄ is A, X₃₅ is P, X₃₆ is V, X₃₇ is P, X₃₈ is Q, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No70 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is V, X₁₉ is P, X₂₀ is L, X₂₁ is P, X₂₂ is T, X₂₃ is L, X₂₄ is P, X₂₅ is M, X₂₆ is A, X₂₇ is T, X₂₈ is A, X₂₉ is W, X₃₀ is G, X₃₁ is Q, X₃₂ is H, X₃₃ is A, X₃₄ is A, X₃₅ is P, X₃₆ is V, X₃₇ is P, X₃₈ is Q, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No71 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is V, X₁₉ is P, X₂₀ is L, X₂₁ is P, X₂₂ is T, X₂₃ is L, X₂₄ is P, X₂₅ is M, X₂₆ is A, X₂₇ is T, X₂₈ is A, X₂₉ is W, X₃₀ is G, X₃₁ is Q, X₃₂ is H, X₃₃ is A, X₃₄ is A, X₃₅ is P, X₃₆ is I, X₃₇ is P, X₃₈ is Q, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No72 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is V, X₁₉ is P, X₂₀ is L, X₂₁ is P, X₂₂ is T, X₂₃ is L, X₂₄ is P, X₂₅ is M, X₂₆ is A, X₂₇ is T, X₂₈ is A, X₂₉ is W, X₃₀ is G, X₃₁ is Q, X₃₂ is H, X₃₃ is A, X₃₄ is A, X₃₅ is P, X₃₆ is V, X₃₇ is P, X₃₈ is P, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is S, X₅₅ is S and X₅₆ is A,

the polypeptide of sequence SEQ ID No73 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is V, X₁₉ is P, X₂₀ is L, X₂₁ is P, X₂₂ is T, X₂₃ is L, X₂₄ is P, X₂₅ is M, X₂₆ is A, X₂₇ is T, X₂₈ is A, X₂₉ is W, X₃₀ is G, X₃₁ is Q, X₃₂ is H, X₃₃ is V, X₃₄ is A, X₃₅ is P, X₃₆ is V, X₃₇ is P, X₃₈ is Q, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No74 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is P, X₂₅ is M, X₂₆ is A, X₂₇ is T, X₂₈ is A, X₂₉ is W, X₃₀ is G, X₃₁ is Q, X₃₂ is H, X₃₃ is V, X₃₄ is A, X₃₅ is P, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No75 which corresponds to the sequence SEQ ID No1 in which X₁ is G, X₂ is A, X₃ is R, X₄ is L, X₅ is G, X₆ is R, X₇ is L, X₈ is V, X₉ is E, X₁O is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is P, X₂₅ is M, X₂₆ is A, X₂₇ is T, X₂₈ is A, X₂₉ is W, X₃₀ is G, X₃₁ is Q, X₃₂ is H, X₃₃ is V, X₃₄ is A, X₃₅ is P, X₃₆ is V, X₃₇ is P, X₃₈ is V, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No76 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is P, X₂₅ is M, X₂₆ is A, X₂₇ is T, X₂₈ is A, X₂₉ is W, X₃₀ is G, X₃₁ is Q, X₃₂ is H, X₃₃ is A, X₃₄ is A, X₃₅ is P, X₃₆ is V, X₃₇ is P, X₃₈ is T, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is L and X₅₆ is A,

the polypeptide of sequence SEQ ID No77 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is P, X₂₅ is M, X₂₆ is A, X₂₇ is T, X₂₈ is A, X₂₉ is W, X₃₀ is G, X₃₁ is Q, X₃₂ is H, X₃₃ is A, X₃₄ is A, X₃₅ is P, X₃₆ is V, X₃₇ is P, X₃₈ is T, X₃₉ is G, X₄₀ is V, X₄₁ is R, X₄₂ is V, X₄₃ is I, X₄₄ is R, X₄₅ is I, X₄₆ is P, X₄₇ is L, X₄₈ is H, X₄₉ is A, X₅₀ is A, X₅₁ is S, X₅₂ is T, X₅₃ is T, X₅₄ is F, X₅₅ is L and X₅₆ is A,

the polypeptide of sequence SEQ ID No78 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is L, X₂₁ is P, X₂₂ is T, X₂₃ is L, X₂₄ is P, X₂₅ is M, X₂₆ is A, X₂₇ is T, X₂₈ is A, X₂₉ is W, X₃₀ is G, X₃₁ is Q, X₃₂ is H, X₃₃ is A, X₃₄ is A, X₃₅ is P, X₃₆ is V, X₃₇ is P, X₃₈ is T, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is L and X₅₆ is A,

the polypeptide of sequence SEQ ID No79 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is L, X₂₁ is P, X₂₂ is T, X₂₃ is L, X₂₄ is P, X₂₅ is M, X₂₆ is A, X₂₇ is T, X₂₈ is A, X₂₉ is W, X₃₀ is G, X₃₁ is Q, X₃₂ is H, X₃₃ is A, X₃₄ is A, X₃₅ is P, X₃₆ is V, X₃₇ is P, X₃₈ is M, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is L and X₅₆ is A,

the polypeptide of sequence SEQ ID No80 which corresponds to the sequence SEQ ID No1 in which X₁ is G, X₂ is A, X₃ is R, X₄ is L, X₅ is G, X₆ is H, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is P, X₂₅ is M, X₂₆ is A, X₂₇ is T, X₂₈ is A, X₂₉ is W, X₃₀ is G, X₃₁ is Q, X₃₂ is H, X₃₃ is A, X₃₄ is A, X₃₅ is P, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is L and X₅₆ is A,

the polypeptide of sequence SEQ ID No81 which corresponds to the sequence SEQ ID No1 in which X₁ is G, X₂ is A, X₃ is R, X₄ is L, X₅ is G, X₆ is H, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is V, X₁₉ is P, X₂₀ is L, X₂₁ is P, X₂₂ is T, X₂₃ is L, X₂₄ is P, X₂₅ is M, X₂₆ is A, X₂₇ is T, X₂₈ is A, X₂₉ is W, X₃₀ is G, X₃₁ is Q, X₃₂ is H, X₃₃ is A, X₃₄ is A, X₃₅ is P, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No82 which corresponds to the sequence SEQ ID No1 in which X₁ is G, X₂ is A, X₃ is R, X₄ is L, X₅ is G, X₆ is H, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is L, X₂₁ is P, X₂₂ is T, X₂₃ is L, X₂₄ is P, X₂₅ is M, X₂₆ is A, X₂₇ is T, X₂₈ is A, X₂₉ is W, X₃₀ is G, X₃₁ is Q, X₃₂ is H, X₃₃ is A, X₃₄ is A, X₃₅ is P, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No83 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is P, X₂₅ is M, X₂₆ is V, X₂₇ is T, X₂₈ is A, X₂₉ is W, X₃₀ is G, X₃₁ is Q, X₃₂ is H, X₃₃ is A, X₃₄ is A, X₃₅ is P, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No84 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is P, X₂₅ is M, X₂₆ is A, X₂₇ is T, X₂₈ is A, X₂₉ is W, X₃₀ is G, X₃₁ is Q, X₃₂ is H, X₃₃ is A, X₃₄ is A, X₃₅ is P, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No85 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is P, X₂₅ is M, X₂₆ is A, X₂₇ is T, X₂₈ is A, X₂₉ is W, X₃₀ is G, X₃₁ is Q, X₃₂ is P, X₃₃ is A, X₃₄ is A, X₃₅ is P, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No86 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is L, X₂₁ is P, X₂₂ is T, X₂₃ is L, X₂₄ is P, X₂₅ is M, X₂₆ is A, X₂₇ is T, X₂₈ is A, X₂₉ is W, X₃₀ is G, X₃₁ is Q, X₃₂ is H, X₃₃ is V, X₃₄ is A, X₃₅ is L, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is L and X₅₆ is A,

the polypeptide of sequence SEQ ID No87 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is L, X₂₁ is P, X₂₂ is T, X₂₃ is L, X₂₄ is P, X₂₅ is M, X₂₆ is A, X₂₇ is T, X₂₈ is A, X₂₉ is W, X₃₀ is G, X₃₁ is Q, X₃₂ is H, X₃₃ is V, X₃₄ is A, X₃₅ is P, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No88 which corresponds to the sequence SEQ ID No1 in which X₁ is G, X₂ is A, X₃ is Q, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is P, X₂₁ is P, X₂₂ is T, X₂₃ is L, X₂₄ is P, X₂₅ is M, X₂₆ is V, X₂₇ is T, X₂₈ is A, X₂₉ is W, X₃₀ is D, X₃₁ is Q, X₃₂ is H, X₃₃ is V, X₃₄ is A, X₃₅ is L, X₃₆ is V, X₃₇ is P, X₃₈ is L, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No89 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is S, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is P, X₂₅ is M, X₂₆ is V, X₂₇ is T, X₂₈ is A, X₂₉ is W, X₃₀ is G, X₃₁ is Q, X₃₂ is H, X₃₃ is A, X₃₄ is A, X₃₅ is L, X₃₆ is I, X₃₇ is L, X₃₈ is Q, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No90 which corresponds to the sequence SEQ ID No1 in which X₁ is G, X₂ is A, X₃ is R, X₄ is L, X₅ is G, X₆ is R, X₇ is L, X₈ is V, X₉ is V, X₁₀ is G, X₁₁ is D, X₁₂ is N, X₁₃ is S, X₁₄ is P, X₁₅ is L, X₁₆ is A, X₁₇ is D, X₁₈ is A, X₁₉ is P, X₂₀ is L, X₂₁ is P, X₂₂ is T, X₂₃ is L, X₂₄ is P, X₂₅ is M, X₂₆ is V, X₂₇ is I, X₂₈ is A, X₂₉ is W, X₃₀ is G, X₃₁ is Q, X₃₂ is P, X₃₃ is V, X₃₄ is A, X₃₅ is L, X₃₆ is V, X₃₇ is P, X₃₈ is M, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is V, X₅₁ is S, X₅₂ is T, X₅₃ is T, X₅₄ is F, X₅₅ is S and X₅₆ is A,

the polypeptide of sequence SEQ ID No91 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is L, X₂₁ is P, X₂₂ is I, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is V, X₃₄ is A, X₃₅ is L, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No92 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is L, X₂₁ is P, X₂₂ is I, X₂₃ is P, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is V, X₃₄ is A, X₃₅ is L, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No93 which corresponds to the sequence SEQ ID No1 in which X₁ is G, X₂ is A, X₃ is R, X₄ is L, X₅ is G, X₆ is R, X₇ is L, X₈ is A, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is L, X₂₁ is P, X₂₂ is T, X₂₃ is H, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is V, X₃₄ is A, X₃₅ is L, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No94 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is V, X₃₄ is A, X₃₅ is L, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No95 which corresponds to the sequence SEQ ID No1 in which X₁ is G, X₂ is A, X₃ is R, X₄ is R, X₅ is G, X₆ is R, X₇ is L, X₈ is L, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is L, X₂₁ is P, X₂₂ is T, X₂₃ is L, X₂₄ is P, X₂₅ is M, X₂₆ is A, X₂₇ is T, X₂₈ is A, X₂₉ is W, X₃₀ is G, X₃₁ is Q, X₃₂ is H, X₃₃ is E, X₃₄ is A, X₃₅ is L, X₃₆ is I, X₃₇ is P, X₃₈ is R, X₃₉ is G, X₄₀ is V, X₄₁ is R, X₄₂ is V, X₄₃ is I, X₄₄ is K, X₄₅ is T, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No96 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is L, X₃₆ is I, X₃₇ is P, X₃₈ is R, X₃₉ is G, X₄₀ is V, X₄₁ is R, X₄₂ is V, X₄₃ is I, X₄₄ is K, X₄₅ is T, X₄₆ is P, X₄₇ is S, X₄₈ is H, X₄₉ is A, X₅₀ is A, X₅₁ is S, X₅₂ is T, X₅₃ is T, X₅₄ is S, X₅₅ is S and X₅₆ is A,

the polypeptide of sequence SEQ ID No97 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is V, X₃₄ is A, X₃₅ is L, X₃₆ is V, X₃₇ is P, X₃₈ is R, X₃₉ is G, X₄₀ is V, X₄₁ is R, X₄₂ is V, X₄₃ is I, X₄₄ is K, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No98 which corresponds to the sequence SEQ ID No1 in which X₁ is G, X₂ is A, X₃ is R, X₄ is R, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is P, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is V, X₃₄ is A, X₃₅ is L, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No99 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is L, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is G, X₅₁ is S, X₅₂ is T, X₅₃ is T, X₅₄ is F, X₅₅ is S and X₅₆ is A,

the polypeptide of sequence SEQ ID No100 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is L, X₃₆ is V, X₃₇ is P, X₃₈ is Q, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is G, X₅₁ is S, X₅₂ is T, X₅₃ is T, X₅₄ is F, X₅₅ is S and X₅₆ is G,

the polypeptide of sequence SEQ ID No101 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is F, X₁₆ is A, X₁₇ is S, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is L, X₃₃ is A, X₃₄ is A, X₃₅ is L, X₃₆ is V, X₃₇ is P, X₃₈ is M, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No102 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is L, X₃₃ is A, X₃₄ is A, X₃₅ is H, X₃₆ is V, X₃₇ is P, X₃₈ is M, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No103 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is V, X₃₀ is G, X₃₁ is Q, X₃₂ is H, X₃₃ is A, X₃₄ is A, X₃₅ is L, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No104 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is V, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is L, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No105 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is L, X₂₁ is P, X₂₂ is T, X₂₃ is H, X₂₄ is P, X₂₅ is M, X₂₆ is A, X₂₇ is T, X₂₈ is A, X₂₉ is W, X₃₀ is G, X₃₁ is Q, X₃₂ is P, X₃₃ is V, X₃₄ is A, X₃₅ is L, X₃₆ is I, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No106 which corresponds to the sequence SEQ ID No1 in which X₁ is G, X₂ is A, X₃ is R, X₄ is L, X₅ is G, X₆ is L, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is V, X₁₉ is P, X₂₀ is L, X₂₁ is P, X₂₂ is T, X₂₃ is H, X₂₄ is P, X₂₅ is T, X₂₆ is A, X₂₇ is T, X₂₈ is A, X₂₉ is W, X₃₀ is G, X₃₁ is Q, X₃₂ is P, X₃₃ is V, X₃₄ is A, X₃₅ is P, X₃₆ is A, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No107 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is V, X₁₉ is P, X₂₀ is L, X₂₁ is P, X₂₂ is T, X₂₃ is L, X₂₄ is P, X₂₅ is T, X₂₆ is A, X₂₇ is M, X₂₈ is A, X₂₉ is W, X₃₀ is D, X₃₁ is R, X₃₂ is P, X₃₃ is V, X₃₄ is V, X₃₅ is L, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No108 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is L, X₂₁ is L, X₂₂ is T, X₂₃ is L, X₂₄ is P, X₂₅ is M, X₂₆ is A, X₂₇ is M, X₂₈ is V, X₂₉ is W, X₃₀ is D, X₃₁ is Q, X₃₂ is H, X₃₃ is V, X₃₄ is A, X₃₅ is L, X₃₆ is V, X₃₇ is P, X₃₈ is R, X₃₉ is G, X₄₀ is V, X₄₁ is R, X₄₂ is V, X₄₃ is T, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No109 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is L, X₂₁ is L, X₂₂ is T, X₂₃ is P, X₂₄ is P, X₂₅ is M, X₂₆ is A, X₂₇ is M, X₂₈ is A, X₂₉ is W, X₃₀ is D, X₃₁ is Q, X₃₂ is P, X₃₃ is A, X₃₄ is A, X₃₅ is L, X₃₆ is V, X₃₇ is P, X₃₈ is L, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No110 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is L, X₂₁ is P, X₂₂ is T, X₂₃ is L, X₂₄ is P, X₂₅ is T, X₂₆ is A, X₂₇ is M, X₂₈ is V, X₂₉ is W, X₃₀ is G, X₃₁ is Q, X₃₂ is H, X₃₃ is V, X₃₄ is A, X₃₅ is L, X₃₆ is V, X₃₇ is L, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is S, X₅₅ is S and X₅₆ is A,

the polypeptide of sequence SEQ ID No111 which corresponds to the sequence SEQ ID No1 in which X₁ is G, X₂ is A, X₃ is R, X₄ is L, X₅ is G, X₆ is R, X₇ is P, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is L, X₂₁ is P, X₂₂ is T, X₂₃ is L, X₂₄ is P, X₂₅ is T, X₂₆ is A, X₂₇ is M, X₂₈ is V, X₂₉ is W, X₃₀ is G, X₃₁ is Q, X₃₂ is H, X₃₃ is V, X₃₄ is A, X₃₅ is L, X₃₆ is V, X₃₇ is L, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is S, X₅₅ is S and X₅₆ is A,

the polypeptide of sequence SEQ ID No112 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is I, X₁₈ is V, X₁₉ is P, X₂₀ is L, X₂₁ is P, X₂₂ is T, X₂₃ is L, X₂₄ is P, X₂₅ is T, X₂₆ is A, X₂₇ is M, X₂₈ is A, X₂₉ is W, X₃₀ is G, X₃₁ is Q, X₃₂ is H, X₃₃ is V, X₃₄ is A, X₃₅ is P, X₃₆ is V, X₃₇ is P, X₃₈ is Q, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is S, X₅₅ is S and X₅₆ is A,

the polypeptide of sequence SEQ ID No113 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is V, X₁₉ is P, X₂₀ is L, X₂₁ is P, X₂₂ is T, X₂₃ is L, X₂₄ is P, X₂₅ is T, X₂₆ is P, X₂₇ is M, X₂₈ is A, X₂₉ is W, X₃₀ is G, X₃₁ is Q, X₃₂ is H, X₃₃ is V, X₃₄ is A, X₃₅ is P, X₃₆ is V, X₃₇ is P, X₃₈ is P, X₃₉ is G, X₄₀ is V, X₄₁ is R, X₄₂ is V, X₄₃ is I, X₄₄ is R, X₄₅ is T, X₄₆ is P, X₄₇ is P, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No114 which corresponds to the sequence SEQ ID No1 in which X₁ is G, X₂ is A, X₃ is Q, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is I, X₁₈ is A, X₁₉ is P, X₂₀ is L, X₂₁ is P, X₂₂ is T, X₂₃ is L, X₂₄ is P, X₂₅ is T, X₂₆ is A, X₂₇ is M, X₂₈ is A, X₂₉ is W, X₃₀ is G, X₃₁ is Q, X₃₂ is H, X₃₃ is V, X₃₄ is A, X₃₅ is L, X₃₆ is V, X₃₇ is P, X₃₈ is M, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is L, X₅₂ is T, X₅₃ is T, X₅₄ is F, X₅₅ is S and X₅₆ is A,

the polypeptide of sequence SEQ ID No115 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is T, X₂₀ is L, X₂₁ is P, X₂₂ is I, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is V, X₃₄ is A, X₃₅ is P, X₃₆ is V, X₃₇ is P, X₃₈ is Q, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No116 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is D, X₁₈ is A, X₁₉ is T, X₂₀ is L, X₂₁ is P, X₂₂ is I, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is P, X₃₆ is V, X₃₇ is P, X₃₈ is Q, X₃₉ is G, X₄₀ is V, X₄₁ is R, X₄₂ is V, X₄₃ is I, X₄₄ is R, X₄₅ is I, X₄₆ is P, X₄₇ is S, X₄₈ is R, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No117 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is T, X₂₀ is L, X₂₁ is P, X₂₂ is I, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is P, X₃₆ is V, X₃₇ is P, X₃₈ is Q, X₃₉ is G, X₄₀ is V, X₄₁ is R, X₄₂ is V, X₄₃ is F, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No118 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is L, X₂₁ is P, X₂₂ is T, X₂₃ is P, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is P, X₃₆ is V, X₃₇ is P, X₃₈ is K, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No119 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is N, X₁₈ is A, X₁₉ is S, X₂₀ is L, X₂₁ is P, X₂₂ is T, X₂₃ is P, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is P, X₃₆ is V, X₃₇ is P, X₃₈ is Q, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No120 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is V, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is P, X₃₃ is A, X₃₄ is A, X₃₅ is P, X₃₆ is V, X₃₇ is P, X₃₈ is Q, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No121 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is P, X₃₆ is I, X₃₇ is P, X₃₈ is Q, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No122 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is F, X₁₄ is P, X₁₅ is L, X₁₆ is A, X₁₇ is S, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is V, X₃₄ is A, X₃₅ is P, X₃₆ is V, X₃₇ is P, X₃₈ is Q, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No123 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is V, X₂₉ is W, X₃₀ is G, X₃₁ is Q, X₃₂ is H, X₃₃ is V, X₃₄ is A, X₃₅ is P, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No124 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is L, X₂₁ is P, X₂₂ is T, X₂₃ is L, X₂₄ is P, X₂₅ is M, X₂₆ is A, X₂₇ is T, X₂₈ is V, X₂₉ is W, X₃₀ is G, X₃₁ is Q, X₃₂ is H, X₃₃ is V, X₃₄ is A, X₃₅ is P, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No125 which corresponds to the sequence SEQ ID No1 in which X₁ is G, X₂ is A, X₃ is R, X₄ is P, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is V, X₂₉ is W, X₃₀ is G, X₃₁ is Q, X₃₂ is H, X₃₃ is T, X₃₄ is A, X₃₅ is P, X₃₆ is G, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is G,

the polypeptide of sequence SEQ ID No126 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, Xi₉ is P, X₂₀ is L, X₂₁ is P, X₂₂ is T, X₂₃ is L, X₂₄ is P, X₂₅ is M, X₂₆ is A, X₂₇ is M, X₂₈ is V, X₂₉ is W, X₃₀ is G, X₃₁ is Q, X₃₂ is H, X₃₃ is T, X₃₄ is A, X₃₅ is P, X₃₆ is G, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No127 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is V, X₁₉ is P, X₂₀ is L, X₂₁ is P, X₂₂ is T, X₂₃ is L, X₂₄ is P, X₂₅ is M, X₂₆ is A, X₂₇ is M, X₂₈ is V, X₂₉ is W, X₃₀ is G, X₃₁ is Q, X₃₂ is H, X₃₃ is T, X₃₄ is A, X₃₅ is P, X₃₆ is G, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No128 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is L, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is L, X₄₈ is H, X₄₉ is A, X₅₀ is A, X₅₁ is S, X₅₂ is T, X₅₃ is T, X₅₄ is S, X₅₅ is S and X₅₆ is A,

the polypeptide of sequence SEQ ID No129 which corresponds to the sequence SEQ ID No1 in which X₁ is G, X₂ is A, X₃ is R, X₄ is L, X₅ is G, X₆ is H, X₇ is L, X₈ is V, X₉ is A, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is L, X₂₁ is P, X₂₂ is T, X₂₃ is L, X₂₄ is P, X₂₅ is M, X₂₆ is A, X₂₇ is T, X₂₈ is A, X₂₉ is W, X₃₀ is G, X₃₁ is Q, X₃₂ is H, X₃₃ is V, X₃₄ is A, X₃₅ is L, X₃₆ is V, X₃₇ is P, X₃₈ is Q, X₃₉ is G, X₄₀ is V, X₄₁ is R, X₄₂ is V, X₄₃ is I, X₄₄ is R, X₄₅ is I, X₄₆ is P, X₄₇ is L, X₄₈ is H, X₄₉ is A, X₅₀ is A, X₅₁ is S, X₅₂ is T, X₅₃ is T, X₅₄ is S, X₅₅ is S and X₅₆ is A,

the polypeptide of sequence SEQ ID No130 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is V, X₃₄ is A, X₃₅ is P, X₃₆ is V, X₃₇ is P, X₃₈ is R, X₃₉ is G, X₄₀ is V, X₄₁ is R, X₄₂ is A, X₄₃ is I, X₄₄ is R, X₄₅ is I, X₄₆ is P, X₄₇ is L, X₄₈ is H, X₄₉ is A, X₅₀ is A, X₅₁ is S, X₅₂ is T, X₅₃ is T, X₅₄ is S, X₅₅ is S and X₅₆ is V,

the polypeptide of sequence SEQ ID No131 which corresponds to the sequence SEQ ID No1 in which X₁ is G, X₂ is A, X₃ is R, X₄ is P, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is N, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is V, X₂₉ is W, X₃₀ is G, X₃₁ is Q, X₃₂ is H, X₃₃ is V, X₃₄ is V, X₃₅ is L, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is L, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No132 which corresponds to the sequence SEQ ID No1 in which X₁ is G, X₂ is A, X₃ is R, X₄ is S, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is D, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is V, X₃₄ is V, X₃₅ is L, X₃₆ is V, X₃₇ is P, X₃₈ is Q, X₃₉ is G, X₄₀ is V, X₄₁ is R, X₄₂ is V, X₄₃ is I, X₄₄ is R, X₄₅ is I, X₄₆ is P, X₄₇ is L, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No133 which corresponds to the sequence SEQ ID No1 in which X₁ is G, X₂ is A, X₃ is R, X₄ is L, X₅ is G, X₆ is H, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is V, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is E, X₃₄ is A, X₃₅ is P, X₃₆ is I, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No134 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is N, X₁₈ is A, X₁₉ is P, X₂₀ is L, X₂₁ is P, X₂₂ is T, X₂₃ is L, X₂₄ is P, X₂₅ is M, X₂₆ is P, X₂₇ is M, X₂₈ is A, X₂₉ is W, X₃₀ is G, X₃₁ is Q, X₃₂ is H, X₃₃ is E, X₃₄ is A, X₃₅ is P, X₃₆ is I, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is L, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No135 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is N, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is V, X₃₄ is A, X₃₅ is L, X₃₆ is I, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No136 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is I, X₁₈ is A, X₁₉ is P, X₂₀ is L, X₂₁ is L, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is V, X₃₄ is A, X₃₅ is L, X₃₆ is V, X₃₇ is P, X₃₈ is R, X₃₉ is G, X₄₀ is V, X₄₁ is R, X₄₂ is A, X₄₃ is I, X₄₄ is R, X₄₅ is I, X₄₆ is P, X₄₇ is S, X₄₈ is R, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No137 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is V, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is V, X₃₄ is A, X₃₅ is L, X₃₆ is V, X₃₇ is P, X₃₈ is R, X₃₉ is G, X₄₀ is V, X₄₁ is R, X₄₂ is A, X₄₃ is I, X₄₄ is R, X₄₅ is I, X₄₆ is P, X₄₇ is S, X₄₈ is R, X₄₉ is A, X₅₀ is A, X₅₁ is S, X₅₂ is T, X₅₃ is T, X₅₄ is F, X₅₅ is L and X₅₆ is A,

the polypeptide of sequence SEQ ID No138 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is V, X₃₄ is V, X₃₅ is L, X₃₆ is V, X₃₇ is P, X₃₈ is R, X₃₉ is G, X₄₀ is V, X₄₁ is R, X₄₂ is A, X₄₃ is I, X₄₄ is R, X₄₅ is I, X₄₆ is P, X₄₇ is S, X₄₈ is R, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No139 which corresponds to the sequence SEQ ID No1 in which X₁ is G, X₂ is A, X₃ is R, X₄ is L, X₅ is G, X₆ is H, X₇ is L, X₈ is V, X₉ is D, X₁₀ is G, X₁₁ is D, X₁₂ is N, X₁₃ is S, X₁₄ is P, X₁₅ is L, X₁₆ is A, X₁₇ is G, X₁₈ is A, X₁₉ is P, X₂₀ is P, X₂₁ is P, X₂₂ is I, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is V, X₃₄ is A, X₃₅ is P, X₃₆ is V, X₃₇ is P, X₃₈ is R, X₃₉ is G, X₄₀ is V, X₄₁ is R, X₄₂ is A, X₄₃ is I, X₄₄ is R, X₄₅ is I, X₄₆ is P, X₄₇ is S, X₄₈ is R, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No140 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is L, X₂₁ is R, X₂₂ is I, X₂₃ is L, X₂₄ is P, X₂₅ is M, X₂₆ is A, X₂₇ is T, X₂₈ is A, X₂₉ is W, X₃₀ is G, X₃₁ is Q, X₃₂ is H, X₃₃ is V, X₃₄ is A, X₃₅ is L, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is R, X₄₉ is A, X₅₀ is A, X₅₁ is S, X₅₂ is T, X₅₃ is T, X₅₄ is F, X₅₅ is S and X₅₆ is V,

the polypeptide of sequence SEQ ID No141 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is P, X₂₁ is P, X₂₂ is I, X₂₃ is L, X₂₄ is P, X₂₅ is M, X₂₆ is A, X₂₇ is T, X₂₈ is A, X₂₉ is W, X₃₀ is G, X₃₁ is Q, X₃₂ is H, X₃₃ is V, X₃₄ is A, X₃₅ is L, X₃₆ is A, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is R, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No142 which corresponds to the sequence SEQ ID No1 in which X₁ is G, X₂ is A, X₃ is R, X₄ is P, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is D, X₁₈ is A, X₁₉ is P, X₂₀ is L, X₂₁ is P, X₂₂ is I, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is M, X₃₄ is A, X₃₅ is L, X₃₆ is V, X₃₇ is P, X₃₈ is R, X₃₉ is G, X₄₀ is V, X₄₁ is R, X₄₂ is V, X₄₃ is I, X₄₄ is R, X₄₅ is I, X₄₆ is P, X₄₇ is S, X₄₈ is R, X₄₉ is A, X₅₀ is A, X₅₁ is S, X₅₂ is T, X₅₃ is T, X₅₄ is S, X₅₅ is S and X₅₆ is A,

the polypeptide of sequence SEQ ID No143 which corresponds to the sequence SEQ ID No1 in which X₁ is G, X₂ is A, X₃ is R, X₄ is P, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is N, X₁₈ is A, X₁₉ is P, X₂₀ is L, X₂₁ is P, X₂₂ is I, X₂₃ is L, X₂₄ is P, X₂₅ is M, X₂₆ is A, X₂₇ is T, X₂₈ is A, X₂₉ is W, X₃₀ is G, X₃₁ is Q, X₃₂ is H, X₃₃ is A, X₃₄ is A, X₃₅ is L, X₃₆ is I, X₃₇ is P, X₃₈ is R, X₃₉ is G, X₄₀ is V, X₄₁ is R, X₄₂ is A, X₄₃ is I, X₄₄ is R, X₄₅ is I, X₄₆ is P, X₄₇ is S, X₄₈ is R, X₄₉ is A, X₅₀ is A, X₅₁ is S, X₅₂ is T, X₅₃ is T, X₅₄ is S, X₅₅ is S and X₅₆ is V,

the polypeptide of sequence SEQ ID No144 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is V, X₁₉ is P, X₂₀ is L, X₂₁ is P, X₂₂ is T, X₂₃ is P, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is V, X₃₄ is A, X₃₅ is L, X₃₆ is V, X₃₇ is P, X₃₈ is R, X₃₉ is G, X₄₀ is V, X₄₁ is R, X₄₂ is A, X₄₃ is I, X₄₄ is R, X₄₅ is I, X₄₆ is P, X₄₇ is S, X₄₈ is R, X₄₉ is A, X₅₀ is A, X₅₁ is S, X₅₂ is I, X₅₃ is T, X₅₄ is F, X₅₅ is S and X₅₆ is A,

the polypeptide of sequence SEQ ID No145 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is V, X₁₉ is P, X₂₀ is L, X₂₁ is P, X₂₂ is T, X₂₃ is H, X₂₄ is P, X₂₅ is M, X₂₆ is A, X₂₇ is M, X₂₈ is V, X₂₉ is W, X₃₀ is G, X₃₁ is Q, X₃₂ is H, X₃₃ is V, X₃₄ is D, X₃₅ is P, X₃₆ is V, X₃₇ is P, X₃₈ is R, X₃₉ is G, X₄₀ is V, X₄₁ is R, X₄₂ is A, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No146 which corresponds to the sequence SEQ ID No1 in which X₁ is G, X₂ is A, X₃ is R, X₄ is L, X₅ is G, X₆ is R, X₇ is L, X₈ is V, X₉ is A, X₁₀ is G, X₁₁ is D, X₁₂ is N, X₁₃ is S, X₁₄ is P, X₁₅ is L, X₁₆ is A, X₁₇ is G, X₁₈ is V, X₁₉ is P, X₂₀ is L, X₂₁ is P, X₂₂ is T, X₂₃ is L, X₂₄ is P, X₂₅ is M, X₂₆ is P, X₂₇ is M, X₂₈ is A, X₂₉ is W, X₃₀ is G, X₃₁ is R, X₃₂ is H, X₃₃ is A, X₃₄ is A, X₃₅ is P, X₃₆ is V, X₃₇ is P, X₃₈ is R, X₃₉ is G, X₄₀ is V, X₄₁ is R, X₄₂ is A, X₄₃ is I, X₄₄ is R, X₄₅ is I, X₄₆ is P, X₄₇ is S, X₄₈ is H, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No147 which corresponds to the sequence SEQ ID No1 in which X₁ is G, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is S, X₁₈ is A, X₁₉ is P, X₂₀ is L, X₂₁ is P, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is P, X₃₆ is V, X₃₇ is P, X₃₈ is P, X₃₉ is G, X₄₀ is V, X₄₁ is R, X₄₂ is A, X₄₃ is I, X₄₄ is R, X₄₅ is I, X₄₆ is P, X₄₇ is S, X₄₈ is R, X₄₉ is V, X₅₀ is A, X₅₁ is S, X₅₂ is T, X₅₃ is T, X₅₄ is F, X₅₅ is S and X₅₆ is A,

the polypeptide of sequence SEQ ID No148 which corresponds to the sequence SEQ ID No1 in which X₁ is R, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is D, X₁₈ is V, X₁₉ is P, X₂₀ is L, X₂₁ is L, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is P, X₃₆ is V, X₃₇ is P, X₃₈ is R, X₃₉ is G, X₄₀ is A, X₄₁ is R, X₄₂ is A, X₄₃ is I, X₄₄ is R, X₄₅ is I, X₄₆ is P, X₄₇ is S, X₄₈ is R, X₄₉ is A, X₅₀ is A, X₅₁ is S, X₅₂ is T, X₅₃ is T, X₅₄ is F, X₅₅ is S and X₅₆ is A,

the polypeptide of sequence SEQ ID No149 which corresponds to the sequence SEQ ID No1 in which X₁ is R, X₂ is A, X₃ is R, X₄ is L, X₅ is G, X₆ is R, X₇ is L, X₈ is V, X₉ is E, X₁₀ is G, X₁₁ is D, X₁₂ is N, X₁₃ is S, X₁₄ is P, X₁₅ is L, X₁₆ is A, X₁₇ is D, X₁₈ is V, X₁₉ is P, X₂₀ is L, X₂₁ is L, X₂₂ is T, X₂₃ is L, X₂₄ is P, X₂₅ is M, X₂₆ is A, X₂₇ is M, X₂₈ is A, X₂₉ is W, X₃₀ is G, X₃₁ is Q, X₃₂ is H, X₃₃ is A, X₃₄ is A, X₃₅ is P, X₃₆ is V, X₃₇ is P, X₃₈ is R, X₃₉ is G, X₄₀ is G, X₄₁ is R, X₄₂ is A, X₄₃ is I, X₄₄ is R, X₄₅ is I, X₄₆ is P, X₄₇ is S, X₄₈ is R, X₄₉ is A, X₅₀ is A, X₅₁ is S, X₅₂ is T, X₅₃ is T, X₅₄ is F, X₅₅ is S and X₅₆ is A, and

the polypeptide of sequence SEQ ID No150 which corresponds to the sequence SEQ ID No1 in which X₁ is S, X₂ is A, X₃ is R, X₄ is L, X₅ is G, X₆ is R, X₇ is L, X₈ is V, X₉ is G, X₁₀ is G, X₁₁ is D, X₁₂ is N, X₁₃ is S, X₁₄ is P, X₁₅ is L, X₁₆ is A, X₁₇ is N, X₁₈ is V, X₁₉ is P, X₂₀ is L, X₂₁ is P, X₂₂ is T, X₂₃ is L, X₂₄ is P, X₂₅ is M, X₂₆ is A, X₂₇ is T, X₂₈ is A, X₂₉ is W, X₃₀ is G, X₃₁ is Q, X₃₂ is H, X₃₃ is A, X₃₄ is A, X₃₅ is P, X₃₆ is V, X₃₇ is P, X₃₈ is Q, X₃₉ is G, X₄₀ is V, X₄₁ is H, X₄₂ is A, X₄₃ is I, X₄₄ is R, X₄₅ is I, X₄₆ is P, X₄₇ is S, X₄₈ is R, X₄₉ is V, X₅₀ is A, X₅₁ is S, X₅₂ is T, X₅₃ is T, X₅₄ is F, X₅₅ is S and X₅₆ is A.

Preferably, the polypeptide F′ of the invention is the polypeptide of sequence SEQ ID No.2.

According to another embodiment of the invention, the polypeptides F′ are derived from genotype 3 of HCV and have the sequence SEQ ID No.151 below:

X₁WVCX₂X₃X₄X₅X₅₇LX₅₈X₅₉X₆₀X₆X₆₁X₇AX₉X₁₀X₁₁X₁₂X₆₂X₁₃PX₆₃X₁₅X₁₆X₁₇X₆₄X₆₅X₁₈X₆₆PGX₂₀SX₂₁GTX₂₃GX₂₄X₆₇X₂₅X₂₆X₂₇RAX₂₉X₃₀X₆₈X₃₁X₆₉GX₇₀CX₃₂X₇₁X₃₃X₃₄X₃₅X₇₂X₇₃X₃₆GX₇₄X₃₇X₃₈TPGX₄₀X₇₅X₄₁AX₄₃X₇₆X₄₄SSX₄₅X₄₆X₄₇X₄₈X₄₉X₅₀ X₅₁X₇₈X₅₂SWGX₅₃X₅₄RSX₇₉X₅₆,

in which

X₁ is D, N, S, Y or G, X₂ is A or V, X₃ is R, Q, K or L, X₄ is R, Y, C, F, H, L or P, X₅ is V, A or T, X₆ is H, R or Q, X₇ is L or P, X₉ is D, V, N, R or T, X₁₀ is G, D or S, X₁₁ is D, V, A, G or E, X₁₂ is S, N or T, X₁₃ is S, P or F, X₁₅ is R, H or L, X₁₆ is V or A, X₁₇ is G, R, E, H or V, X₁₈ is A or D, X₂₀ is L, P or R, X₂₁ is P or L, X₂₃ is L or P, X₂₄ is P or L, X₂₅ is M or T, X₂₆ is V, G, A or E, X₂₇ is M, T or I, X₂₉ is A or V, X₃₀ is G, V or D, X₃₁ is Q or R, X₃₂ is P or L, X₃₃ is A or V, X₃₄ is A or V, X₃₅ is P or L, X₃₆ is L, A, V, R, I or P, X₃₇ is Q, K or P, X₃₈ is M or T, X₄₀ is V, G, D, E or A, X₄₁ is P, H or L, X₄₃ is I or T, X₄₄ is R or K, X₄₅ is I or T, X₄₆ is P or L, X₄₇ is S or L, X₄₈ is R or H, X₄₉ is A or V, X₅₀ is D, G, A or V, X₅₁ is S or L, X₅₂ is T, I or A, X₅₃ is T or I, X₅₄ is F or S, X₅₆ is A or V, X₅₇ is K, R or N, X₅₈ is L, P or Q, X₅₉ is S or N, X₆₀ is G or D, X₆₁ is S or N, X₆₂ is L or P, X₆₃ is R or G, X₆₄ is A, P or L, X₆₅ is R, K, E or T, X₆₆ is G or D, X₆₇ is S, Y or F, X₆₈ is G or W, X₆₉ is G or D, X₇₀ is S or F, X₇₁ is P, H, R or L, X₇₂ is V, A, D or G, X₇₃ is H, L, P, Q or R, X₇₄ is A or P, X₇₅ is G or D, X₇₆ is W or L, X₇₇ is V or A, X₇₈ is P or L and X₇₉ is S, L or Q.

Preferably, the polypeptide F′ is chosen from the following polypeptides:

the polypeptide of sequence SEQ ID No152 which corresponds to the sequence SEQ ID No151 in which X₁ is D, X₂ is A, X₃ is R, X₄ is R, X₅ is V, X₆ is H, X₇ is L, X₉ is D, X₁₀ is G, X₁₁ is D, X₁₂ is S, X₁₃ is S, X₁₅ is R, X₁₆ is V, X₁₇ is G, X₁₈ is A, X₂₀ is L, X₂₁ is P, X₂₃ is L, X₂₄ is P, X₂₅ is M, X₂₆ is V, X₂₇ is M, X₂₉ is A, X₃₀ is G, X₃₁ is Q, X₃₂ is P, X₃₃ is A, X₃₄ is A, X₃₅ is P, X₃₆ is L, X₃₇ is Q, X₃₈ is M, X₄₀ is V, X₄₁ is P, X₄₃ is I, X₄₄ is R, X₄₅ is I, X₄₆ is P, X₄₇ is L, X₄₈ is R, X₄₉ is A, X₅₀ is D, X₅₁ is S, X₅₂ is T, X₅₃ is T, X₅₄ is F, X₅₆ is A, X₅₇ is K, X₅₈ is L, X₅₉ is S, X₆₀ is G, X₆₁ is S, X₆₂ is L, X₆₃ is R, X₆₄ is A, X₆₅ is R, X₆₆ is G, X₆₇ is S, X₆₈ is G, X₆₉ is G, X₇₀ is S, X₇₁ is P, X₇₂ is V, X₇₃ is H, X₇₄ is A, X₇₅ is G, X₇₆ is W, X₇₇ is V, X₇₈ is P and X₇₉ is S,

the polypeptide of sequence SEQ ID No153 which corresponds to the sequence SEQ ID No151 in which X₁ is D, X₂ is A, X₃ is R, X₄ is R, X₅ is V, X₆ is H, X₇ is L, X₉ is D, X₁₀ is D, X₁₁ is D, X₁₂ is S, X₁₃ is S, X₁₅ is R, X₁₆ is V, X₁₇ is G, X₁₈ is A, X₂₀ is L, X₂₁ is P, X₂₃ is L, X₂₄ is P, X₂₅ is M, X₂₆ is V, X₂₇ is T, X₂₉ is A, X₃₀ is G, X₃₁ is Q, X₃₂ is P, X₃₃ is A, X₃₄ is A, X₃₅ is P, X₃₆ is P, X₃₇ is Q, X₃₈ is M, X₄₀ is G, X₄₁ is P, X₄₃ is I, X₄₄ is K, X₄₅ is I, X₄₆ is P, X₄₇ is L, X₄₈ is R, X₄₉ is A, X₅₀ is D, X₅₁ is S, X₅₂ is T, X₅₃ is T, X₅₄ is S, X₅₆ is A, X₅₇ is K, X₅₈ is L, X₅₉ is N, X₆₀ is G, X₆₁ is S, X₆₂ is L, X₆₃ is R, X₆₄ is A, X₆₅ is K, X₆₆ is G, X₆₇ is S, X₆₈ is G, X₆₉ is G, X₇₀ is S, X₇₁ is H, X₇₂ is V, X₇₃ is H, X₇₄ is A, X₇₅ is G, X₇₆ is W, X₇₇ is V, X₇₈ is P and X₇₉ is S.

the polypeptide of sequence SEQ ID No154 which corresponds to the sequence SEQ ID No151 in which X₁ is D, X₂ is A, X₃ is R, X₄ is P, X₅ is V, X₆ is H, X₇ is L, X₉ is D, X₁₀ is D, X₁₁ is D, X₁₂ is S, X₁₃ is S, X₁₅ is R, X₁₆ is V, X₁₇ is G, X₁₈ is A, X₂₀ is L, X₂₁ is P, X₂₃ is L, X₂₄ is P, X₂₅ is M, X₂₆ is V, X₂₇ is T, X₂₉ is A, X₃₀ is G, X₃₁ is Q, X₃₂ is P, X₃₃ is A, X₃₄ is A, X₃₅ is L, X₃₆ is L, X₃₇ is Q, X₃₈ is M, X₄₀ is G, X₄₁ is P, X₄₃ is I, X₄₄ is K, X₄₅ is I, X₄₆ is P, X₄₇ is L, X₄₈ is R, X₄₉ is A, X₅₀ is D, X₅₁ is S, X₅₂ is T, X₅₃ is T, X₅₄ is S, X₅₆ is A, X₅₇ is K, X₅₈ is L, X₅₉ is N, X₆₀ is G, X₆₁ is S, X₆₂ is L, X₆₃ is R, X₆₄ is A, X₆₅ is T, X₆₆ is G, X₆₇ is S, X₆₈ is G, X₆₉ is G, X₇₀ is S, X₇₁ is H, X₇₂ is V, X₇₃ is H, X₇₄ is A, X₇₅ is G, X₇₆ is W, X₇₇ is V, X₇₈ is P and X₇₉ is S,

the polypeptide of sequence SEQ ID No155 which corresponds to the sequence SEQ ID No151 in which X₁ is D, X₂ is A, X₃ is R, X₄ is R, X₅ is V, X₆ is H, X₇ is P, X₉ is D, X₁₀ is D, X₁₁ is D, X₁₂ is S, X₁₃ is S, X₁₅ is R, X₁₆ is V, X₁₇ is R, X₁₈ is A, X₂₀ is L, X₂₁ is P, X₂₃ is L, X₂₄ is P, X₂₅ is M, X₂₆ is V, X₂₇ is M, X₂₉ is A, X₃₀ is G, X₃₁ is Q, X₃₂ is P, X₃₃ is A, X₃₄ is A, X₃₅ is P, X₃₆ is L, X₃₇ is Q, X₃₈ is M, X₄₀ is G, X₄₁ is P, X₄₃ is I, X₄₄ is K, X₄₅ is I, X₄₆ is P, X₄₇ is L, X₄₈ is R, X₄₉ is V, X₅₀ is D, X₅₁ is S, X₅₂ is T, X₅₃ is T, X₅₄ is S, X₅₆ is A, X₅₇ is K, X₅₈ is L, X₅₉ is N, X₆₀ is G, X₆₁ is S, X₆₂ is L, X₆₃ is R, X₆₄ is A, X₆₅ is E, X₆₆ is G, X₆₇ is S, X₆₈ is G, X₆₉ is G, X₇₀ is S, X₇₁ is R, X₇₂ is V, X₇₃ is H, X₇₄ is A, X₇₅ is G, X₇₆ is W, X₇₇ is V, X₇₈ is P and X₇₉ is S,

the polypeptide of sequence SEQ ID No156 which corresponds to the sequence SEQ ID No151 in which X₁ is D, X₂ is A, X₃ is R, X₄ is R, X₅ is V, X₆ is R, X₇ is L, X₉ is D, X₁₀ is D, X₁₁ is D, X₁₂ is S, X₁₃ is S, X₁₅ is R, X₁₆ is V, X₁₇ is G, X₁₈ is A, X₂₀ is L, X₂₁ is P, X₂₃ is L, X₂₄ is P, X₂₅ is M, X₂₆ is V, X₂₇ is T, X₂₉ is A, X₃₀ is G, X₃₁ is Q, X₃₂ is P, X₃₃ is A, X₃₄ is A, X₃₅ is P, X₃₆ is L, X₃₇ is Q, X₃₈ is T, X₄₀ is D, X₄₁ is P, X₄₃ is I, X₄₄ is K, X₄₅ is I, X₄₆ is P, X₄₇ is L, X₄₈ is R, X₄₉ is A, X₅₀ is D, X₅₁ is S, X₅₂ is T, X₅₃ is T, X₅₄ is S, X₅₆ is A, X₅₇ is K, X₅₈ is L, X₅₉ is N, X₆₀ is G, X₆₁ is S, X₆₂ is L, X₆₃ is R, X₆₄ is A, X₆₅ is K, X₆₆ is G, X₆₇ is S, X₆₈ is G, X₆₉ is D, X₇₀ is S, X₇₁ is H, X₇₂ is V, X₇₃ is H, X₇₄ is A, X₇₅ is G, X₇₆ is W, X₇₇ is V, X₇₈ is P and X₇₉ is S.

the polypeptide of sequence SEQ ID No157 which corresponds to the sequence SEQ ID No151 in which X₁ is D, X₂ is A, X₃ is R, X₄ is R, X₅ is V, X₆ is H, X₇ is L, X₉ is D, X₁₀ is D, X₁₁ is G, X₁₂ is S, X₁₃ is S, X₁₅ is R, X₁₆ is V, X₁₇ is G, X₁₈ is A, X₂₀ is L, X₂₁ is P, X₂₃ is L, X₂₄ is P, X₂₅ is M, X₂₆ is V, X₂₇ is T, X₂₉ is A, X₃₀ is G, X₃₁ is Q, X₃₂ is P, X₃₃ is A, X₃₄ is A, X₃₅ is P, X₃₆ is L, X₃₇ is Q, X₃₈ is M, X₄₀ is D, X₄₁ is P, X₄₃ is I, X₄₄ is K, X₄₅ is I, X₄₆ is P, X₄₇ is L, X₄₈ is R, X₄₉ is A, X₅₀ is D, X₅₁ is S, X₅₂ is T, X₅₃ is T, X₅₄ is S, X₅₆ is A, X₅₇ is K, X₅₈ is L, X₅₉ is N, X₆₀ is G, X₆₁ is S, X₆₂ is L, X₆₃ is R, X₆₄ is A, X₆₅ is K, X₆₆ is G, X₆₇ is S, X₆₈ is G, X₆₉ is D, X₇₀ is S, X₇₁ is R, X₇₂ is V, X₇₃ is H, X₇₄ is A, X₇₅ is G, X₇₆ is W, X₇₇ is V, X₇₈ is P and X₇₉ is S.

the polypeptide of sequence SEQ ID No158 which corresponds to the sequence SEQ ID No151 in which X₁ is D, X₂ is A, X₃ is R, X₄ is R, X₅ is V, X₆ is H, X₇ is L, X₉ is D, X₁₀ is D, X₁₁ is D, X₁₂ is S, X₁₃ is S, X₁₅ is H, X₁₆ is V, X₁₇ is G, X₁₈ is A, X₂₀ is L, X₂₁ is L, X₂₃ is L, X₂₄ is P, X₂₅ is M, X₂₆ is V, X₂₇ is T, X₂₉ is A, X₃₀ is G, X₃₁ is Q, X₃₂ is P, X₃₃ is A, X₃₄ is A, X₃₅ is P, X₃₆ is L, X₃₇ is Q, X₃₈ is T, X₄₀ is D, X₄₁ is P, X₄₃ is I, X₄₄ is K, X₄₅ is I, X₄₆ is P, X₄₇ is L, X₄₈ is R, X₄₉ is A, X₅₀ is D, X₅₁ is S, X₅₂ is T, X₅₃ is T, X₅₄ is S, X₅₆ is A, X₅₇ is K, X₅₈ is L, X₅₉ is N, X₆₀ is G, X₆₁ is S, X₆₂ is L, X₆₃ is R, X₆₄ is A, X₆₅ is K, X₆₆ is G, X₆₇ is S, X₆₈ is G, X₆₉ is G, X₇₀ is S, X₇₁ is H, X₇₂ is V, X₇₃ is H, X₇₄ is A, X₇₅ is G, X₇₆ is W, X₇₇ is V, X₇8 is P and X₇₉ is S,

the polypeptide of sequence SEQ ID No159 which corresponds to the sequence SEQ ID No151 in which X₁ is D, X₂ is A, X₃ is R, X₄ is R, X₅ is V, X₆ is H, X₇ is L, X₉ is D, X₁₀ is D, X₁₁ is D, X₁₂ is S, X₁₃ is S, X₁₅ is R, X₁₆ is V, X₁₇ is G, X₁₈ is A, X₂₀ is L, X₂₁ is P, X₂₃ is L, X₂₄ is P, X₂₅ is M, X₂₆ is V, X₂₇ is T, X₂₉ is A, X₃₀ is G, X₃₁ is Q, X₃₂ is P, X₃₃ is A, X₃₄ is A, X₃₅ is P, X₃₆ is L, X₃₇ is Q, X₃₈ is T, X₄₀ is G, X₄₁ is P, X₄₃ is I, X₄₄ is K, X₄₅ is I, X₄₆ is P, X₄₇ is L, X₄₈ is R, X₄₉ is A, X₅₀ is V, X₅₁ is S, X₅₂ is T, X₅₃ is T, X₅₄ is S, X₅₆ is A, X₅₇ is K, X₅₈ is L, X₅₉ is N, X₆₀ is G, X₆₁ is S, X₆₂ is L, X₆₃ is R, X₆₄ is A, X₆₅ is K, X₆₆ is G, X₆₇ is S, X₆₈ is G, X₆₉ is G, X₇₀ is S, X₇₁ is H, X₇₂ is V, X₇₃ is Q, X₇₄ is A, X₇₅ is G, X₇₆ is W, X₇₇ is V, X₇₈ is P and X₇₉ is S,

the polypeptide of sequence SEQ ID No160 which corresponds to the sequence SEQ ID No151 in which X₁ is D, X₂ is A, X₃ is R, X₄ is R, X₅ is V, X₆ is H, X₇ is L, X₉ is D, X₁₀ is D, X₁₁ is D, X₁₂ is S, X₁₃ is S, X₁₅ is R, X₁₆ is V, X₁₇ is G, X₁₈ is A, X₂₀ is L, X₂₁ is P, X₂₃ is L, X₂₄ is P, X₂₅ is M, X₂₆ is V, X₂₇ is M, X₂₉ is A, X₃₀ is G, X₃₁ is Q, X₃₂ is P, X₃₃ is A, X₃₄ is A, X₃₅ is P, X₃₆ is L, X₃₇ is Q, X₃₈ is T, X₄₀ is G, X₄₁ is P, X₄₃ is I, X₄₄ is K, X₄₅ is I, X₄₆ is P, X₄₇ is L, X₄₈ is R, X₄₉ is A, X₅₀ is D, X₅₁ is S, X₅₂ is T, X₅₃ is T, X₅₄ is S, X₅₆ is A, X₅₇ is K, X₅₈ is L, X₅₉ is N, X₆₀ is G, X₆₁ is S, X₆₂ is L, X₆₃ is R, X₆₄ is A, X₆₅ is K, X₆₆ is G, X₆₇ is S, X₆₈ is G, X₆₉ is G, X₇₀ is S, X₇₁ is H, X₇₂ is V, X₇₃ is H, X₇₄ is A, X₇₅ is G, X₇₆ is W, X₇₇ is V, X₇₈ is P and X₇₉ is S,

the polypeptide of sequence SEQ ID No161 which corresponds to the sequence SEQ ID No151 in which X₁ is D, X₂ is A, X₃ is R, X₄ is R, X₅ is V, X₆ is H, X₇ is L, X₉ is D, X₁₀ is G, X₁₁ is D, X₁₂ is S, X₁₃ is S, X₁₅ is R, X₁₆ is V, X₁₇ is G, X₁₈ is A, X₂₀ is L, X₂₁ is P, X₂₃ is L, X₂₄ is P, X₂₅ is M, X₂₆ is V, X₂₇ is T, X₂₉ is A, X₃₀ is G, X₃₁ is Q, X₃₂ is P, X₃₃ is A, X₃₄ is A, X₃₅ is P, X₃₆ is L, X₃₇ is Q, X₃₈ is M, X₄₀ is G, X₄₁ is P, X₄₃ is I, X₄₄ is K, X₄₅ is I, X₄₆ is P, X₄₇ is L, X₄₈ is R, X₄₉ is A, X₅₀ is A, X₅₁ is S, X₅₂ is T, X₅₃ is T, X₅₄ is S, X₅₆ is A, X₅₇ is K, X₅₈ is L, X₅₉ is N, X₆₀ is G, X₆₁ is S, X₆₂ is L, X₆₃ is R, X₆₄ is A, X₆₅ is K, X₆₆ is D, X₆₇ is S, X₆₈ is G, X₆₉ is G, X₇₀ is S, X₇₁ is H, X₇₂ is V, X₇₃ is H, X₇₄ is A, X₇₅ is G, X₇₆ is W, X₇₇ is V, X₇₈ is P and X₇₉ is S,

the polypeptide of sequence SEQ ID No162 which corresponds to the sequence SEQ ID No151 in which X₁ is D, X₂ is A, X₃ is R, X₄ is R, X₅ is V, X₆ is H, X₇ is L, X₉ is D, X₁₀ is G, X₁₁ is D, X₁₂ is S, X₁₃ is S, X₁₅ is R, X₁₆ is V, X₁₇ is G, X₁₈ is A, X₂₀ is L, X₂₁ is P, X₂₃ is L, X₂₄ is P, X₂₅ is M, X₂₆ is V, X₂₇ is T, X₂₉ is A, X₃₀ is G, X₃₁ is Q, X₃₂ is P, X₃₃ is A, X₃₄ is A, X₃₅ is L, X₃₆ is L, X₃₇ is K, X₃₈ is M, X₄₀ is G, X₄₁ is P, X₄₃ is T, X₄₄ is R, X₄₅ is I, X₄₆ is P, X₄₇ is L, X₄₈ is R, X₄₉ is A, X₅₀ is D, X₅₁ is S, X₅₂ is T, X₅₃ is T, X₅₄ is S, X₅₆ is A, X₅₇ is K, X₅₈ is L, X₅₉ is N, X₆₀ is G, X₆₁ is S, X₆₂ is L, X₆₃ is R, X₆₄ is A, X₆₅ is K, X₆₆ is G, X₆₇ is S, X₆₈ is G, X₆₉ is G, X₇₀ is S, X₇₁ is R, X₇₂ is V, X₇₃ is H, X₇₄ is A, X₇₅ is G, X₇₆ is W, X₇₇ is A, X₇₈ is P and X₇₉ is S,

the polypeptide of sequence SEQ ID No163 which corresponds to the sequence SEQ ID No151 in which X₁ is D, X₂ is A, X₃ is R, X₄ is R, X₅ is V, X₆ is H, X₇ is L, X₉ is D, X₁₀ is G, X₁₁ is D, X₁₂ is S, X₁₃ is S, X₁₅ is R, X₁₆ is V, X₁₇ is G, X₁₈ is A, X₂₀ is L, X₂₁ is L, X₂₃ is L, X₂₄ is P, X₂₅ is M, X₂₆ is V, X₂₇ is T, X₂₉ is A, X₃₀ is G, X₃₁ is Q, X₃₂ is P, X₃₃ is A, X₃₄ is A, X₃₅ is P, X₃₆ is L, X₃₇ is Q, X₃₈ is T, X₄₀ is G, X₄₁ is P, X₄₃ is I, X₄₄ is R, X₄₅ is I, X₄₆ is P, X₄₇ is S, X₄₈ is R, X₄₉ is A, X₅₀ is D, X₅₁ is S, X₅₂ is T, X₅₃ is T, X₅₄ is S, X₅₆ is A, X₅₇ is K, X₅₈ is L, X₅₉ is N, X₆₀ is G, X₆₁ is S, X₆₂ is L, X₆₃ is R, X₆₄ is A, X₆₅ is K, X₆₆ is G, X₆₇ is S, X₆₈ is G, X₆₉ is G, X₇₀ is S, X₇₁ is H, X₇₂ is V, X₇₃ is H, X₇₄ is A, X₇₅ is G, X₇₆ is W, X₇₇ is V, X₇₈ is P and X₇₉ is S,

the polypeptide of sequence SEQ ID No164 which corresponds to the sequence SEQ ID No151 in which X₁ is D, X₂ is A, X₃ is R, X₄ is R, X₅ is V, X₆ is R, X₇ is L, X₉ is D, X₁₀ is G, X₁₁ is E, X₁₂ is S, X₁₃ is S, X₁₅ is R, X₁₆ is V, X₁₇ is G, X₁₈ is A, X₂₀ is P, X₂₁ is L, X₂₃ is L, X₂₄ is P, X₂₅ is M, X₂₆ is V, X₂₇ is M, X₂₉ is A, X₃₀ is G, X₃₁ is Q, X₃₂ is P, X₃₃ is A, X₃₄ is A, X₃₅ is P, X₃₆ is I, X₃₇ is Q, X₃₈ is M, X₄₀ is D, X₄₁ is P, X₄₃ is I, X₄₄ is K, X₄₅ is I, X₄₆ is P, X₄₇ is S, X₄₈ is R, X₄₉ is A, X₅₀ is D, X₅₁ is S, X₅₂ is I, X₅₃ is T, X₅₄ is S, X₅₆ is A, X₅₇ is K, X₅₈ is L, X₅₉ is N, X₆₀ is G, X₆₁ is S, X₆₂ is L, X₆₃ is R, X₆₄ is A, X₆₅ is K, X₆₆ is G, X₆₇ is S, X₆₈ is G, X₆₉ is G, X₇₀ is S, X₇₁ is H, X₇₂ is V, X₇₃ is Q, X₇₄ is P, X₇₅ is G, X₇₆ is W, X₇₇ is V, X₇₈ is P and X₇₉ is S,

the polypeptide of sequence SEQ ID No165 which corresponds to the sequence SEQ ID No151 in which X₁ is D, X₂ is A, X₃ is Q, X₄ is L, X₅ is V, X₆ is H, X₇ is P, X₉ is D, X₁₀ is G, X₁₁ is D, X₁₂ is S, X₁₃ is F, X₁₅ is H, X₁₆ is A, X₁₇ is R, X₁₈ is D, X₂₀ is R, X₂₁ is L, X₂₃ is P, X₂₄ is P, X₂₅ is M, X₂₆ is V, X₂₇ is T, X₂₉ is A, X₃₀ is G, X₃₁ is Q, X₃₂ is L, X₃₃ is A, X₃₄ is A, X₃₅ is P, X₃₆ is V, X₃₇ is P, X₃₈ is T, X₄₀ is E, X₄₁ is P, X₄₃ is I, X₄₄ is R, X₄₅ is T, X₄₆ is L, X₄₇ is S, X₄₈ is H, X₄₉ is A, X₅₀ is G, X₅₁ is S, X₅₂ is I, X₅₃ is I, X₅₄ is F, X₅₆ is A, X₅₇ is R, X₅₈ is Q, X₅₉ is S, X₆₀ is G, X₆₁ is N, X₆₂ is L, X₆₃ is R, X₆₄ is A, X₆₅ is E, X₆₆ is G, X₆₇ is S, X₆₈ is G, X₆₉ is G, X₇₀ is F, X₇₁ is L, X₇₂ is D, X₇₃ is R, X₇₄ is A, X₇₅ is G, X₇₆ is W, X₇₇ is V, X₇₈ is P and X₇₉ is S,

the polypeptide of sequence SEQ ID No166 which corresponds to the sequence SEQ ID No151 in which X₁ is D, X₂ is A, X₃ is Q, X₄ is F, X₅ is V, X₆ is Q, X₇ is P, X₉ is T, X₁₀ is G, X₁₁ is G, X₁₂ is S, X₁₃ is S, X₁₅ is H, X₁₆ is V, X₁₇ is G, X₁₈ is A, X₂₀ is L, X₂₁ is L, X₂₃ is L, X₂₄ is L, X₂₅ is M, X₂₆ is A, X₂₇ is M, X₂₉ is V, X₃₀ is G, X₃₁ is Q, X₃₂ is P, X₃₃ is A, X₃₄ is A, X₃₅ is L, X₃₆ is L, X₃₇ is P, X₃₈ is M, X₄₀ is E, X₄₁ is L, X₄₃ is T, X₄₄ is K, X₄₅ is I, X₄₆ is P, X₄₇ is L, X₄₈ is R, X₄₉ is A, X₅₀ is D, X₅₁ is S, X₅₂ is I, X₅₃ is T, X₅₄ is F, X₅₆ is A, X₅₇ is K, X₅₈ is P, X₅₉ is S, X₆₀ is G, X₆₁ is N, X₆₂ is L, X₆₃ is R, X₆₄ is A, X₆₅ is R, X₆₆ is G, X₆₇ is F, X₆₈ is G, X₆₉ is D, X₇₀ is S, X₇₁ is P, X₇₂ is G, X₇₃ is H, X₇₄ is A, X₇₅ is G, X₇₆ is W, X₇₇ is V, X₇8 is P and X₇₉ is S,

the polypeptide of sequence SEQ ID No167 which corresponds to the sequence SEQ ID No151 in which X₁ is Y, X₂ is A, X₃ is L, X₄ is H, X₅ is V, X₆ is H, X₇ is L, X₉ is R, X₁₀ is G, X₁₁ is G, X₁₂ is S, X₁₃ is S, X₁₅ is R, X₁₆ is V, X₁₇ is V, X₁₈ is D, X₂₀ is L, X₂₁ is P, X₂₃ is L, X₂₄ is P, X₂₅ is T, X₂₆ is V, X₂₇ is M, X₂₉ is A, X₃₀ is G, X₃₁ is R, X₃₂ is P, X₃₃ is A, X₃₄ is V, X₃₅ is L, X₃₆ is A, X₃₇ is Q, X₃₈ is M, X₄₀ is E, X₄₁ is P, X₄₃ is I, X₄₄ is R, X₄₅ is I, X₄₆ is P, X₄₇ is L, X₄₈ is H, X₄₉ is A, X₅₀ is G, X₅₁ is S, X₅₂ is T, X₅₃ is T, X₅₄ is F, X₅₆ is A, X₅₇ is R, X₅₈ is P, X₅₉ is S, X₆₀ is G, X₆₁ is S, X₆₂ is L, X₆₃ is R, X₆₄ is A, X₆₅ is R, X₆₆ is G, X₆₇ is S, X₆₈ is G, X₆₉ is D, X₇₀ is S, X₇₁ is P, X₇₂ is G, X₇₃ is R, X₇₄ is A, X₇₅ is D, X₇₆ is W, X₇₇ is V, X₇₈ is P and X₇₉ is S,

the polypeptide of sequence SEQ ID No168 which corresponds to the sequence SEQ ID No151 in which X₁ is D, X₂ is A, X₃ is Q, X₄ is Y, X₅ is V, X₆ is R, X₇ is L, X₉ is D, X₁₀ is G, X₁₁ is V, X₁₂ is S, X₁₃ is S, X₁₅ is H, X₁₆ is A, X₁₇ is R, X₁₈ is A, X₂₀ is L, X₂₁ is L, X₂₃ is L, X₂₄ is P, X₂₅ is T, X₂₆ is E, X₂₇ is M, X₂₉ is V, X₃₀ is G, X₃₁ is Q, X₃₂ is P, X₃₃ is A, X₃₄ is V, X₃₅ is L, X₃₆ is I, X₃₇ is Q, X₃₈ is M, X₄₀ is V, X₄₁ is P, X₄₃ is T, X₄₄ is R, X₄₅ is T, X₄₆ is P, X₄₇ is L, X₄₈ is H, X₄₉ is A, X₅₀ is D, X₅₁ is S, X₅₂ is T, X₅₃ is T, X₅₄ is F, X₅₆ is A, X₅₇ is R, X₅₈ is P, X₅₉ is S, X₆₀ is G, X₆₁ is N, X₆₂ is L, X₆₃ is R, X₆₄ is A, X₆₅ is R, X₆₆ is G, X₆₇ is S, X₆₈ is G, X₆₉ is D, X₇₀ is F, X₇₁ is P, X₇₂ is V, X₇₃ is L, X₇₄ is A, X₇₅ is D, X₇₆ is W, X₇₇ is V, X₇₈ is P and X₇₉ is S,

the polypeptide of sequence SEQ ID No169 which corresponds to the sequence SEQ ID No151 in which X₁ is D, X₂ is A, X₃ is Q, X₄ is Y, X₅ is V, X₆ is R, X₇ is L, X₉ is N, X₁₀ is S, X₁₁ is V, X₁₂ is T, X₁₃ is S, X₁₅ is L, X₁₆ is A, X₁₇ is R, X₁₈ is A, X₂₀ is L, X₂₁ is P, X₂₃ is L, X₂₄ is P, X₂₅ is T, X₂₆ is G, X₂₇ is I, X₂₉ is V, X₃₀ is G, X₃₁ is Q, X₃₂ is P, X₃₃ is V, X₃₄ is V, X₃₅ is L, X₃₆ is V, X₃₇ is Q, X₃₈ is M, X₄₀ is V, X₄₁ is P, X₄₃ is T, X₄₄ is R, X₄₅ is I, X₄₆ is P, X₄₇ is L, X₄₈ is R, X₄₉ is V, X₅₀ is D, X₅₁ is S, X₅₂ is T, X₅₃ is T, X₅₄ is F, X₅₆ is A, X₅₇ is R, X₅₈ is P, X₅₉ is S, X₆₀ is G, X₆₁ is S, X₆₂ is L, X₆₃ is R, X₆₄ is A, X₆₅ is R, X₆₆ is G, X₆₇ is S, X₆₈ is W, X₆₉ is D, X₇₀ is S, X₇₁ is P, X₇₂ is A, X₇₃ is L, X₇₄ is A, X₇₅ is D, X₇₆ is L, X₇₇ is V, X₇8 is P and X₇₉ is S,

the polypeptide of sequence SEQ ID No170 which corresponds to the sequence SEQ ID No151 in which X₁ is N, X₂ is A, X₃ is K, X₄ is Y, X₅ is V, X₆ is H, X₇ is L, X₉ is D, X₁₀ is G, X₁₁ is V, X₁₂ is S, X₁₃ is P, X₁₅ is H, X₁₆ is A, X₁₇ is R, X₁₈ is A, X₂₀ is P, X₂₁ is P, X₂₃ is L, X₂₄ is P, X₂₅ is T, X₂₆ is G, X₂₇ is M, X₂₉ is V, X₃₀ is V, X₃₁ is Q, X₃₂ is P, X₃₃ is A, X₃₄ is V, X₃₅ is L, X₃₆ is A, X₃₇ is K, X₃₈ is M, X₄₀ is V, X₄₁ is P, X₄₃ is T, X₄₄ is R, X₄₅ is I, X₄₆ is P, X₄₇ is L, X₄₈ is R, X₄₉ is A, X₅₀ is D, X₅₁ is L, X₅₂ is T, X₅₃ is T, X₅₄ is F, X₅₆ is A, X₅₇ is R, X₅₈ is P, X₅₉ is S, X₆₀ is D, X₆₁ is N, X₆₂ is L, X₆₃ is R, X₆₄ is A, X₆₅ is R, X₆₆ is G, X₆₇ is S, X₆₈ is G, X₆₉ is D, X₇₀ is S, X₇₁ is P, X₇₂ is A, X₇₃ is P, X₇₄ is A, X₇₅ is D, X₇₆ is W, X₇₇ is V, X₇₈ is P and X₇₉ is S,

the polypeptide of sequence SEQ ID No171 which corresponds to the sequence SEQ ID No151 in which X₁ is D, X₂ is A, X₃ is Q, X₄ is Y, X₅ is V, X₆ is R, X₇ is L, X₉ is D, X₁₀ is G, X₁₁ is V, X₁₂ is S, X₁₃ is S, X₁₅ is H, X₁₆ is A, X₁₇ is H, X₁₈ is A, X₂₀ is L, X₂₁ is L, X₂₃ is L, X₂₄ is P, X₂₅ is M, X₂₆ is G, X₂₇ is M, X₂₉ is A, X₃₀ is V, X₃₁ is Q, X₃₂ is P, X₃₃ is V, X₃₄ is V, X₃₅ is L, X₃₆ is V, X₃₇ is Q, X₃₈ is m, X₄₀ is V, X₄₁ is P, X₄₃ is T, X₄₄ is K, X₄₅ is I, X₄₆ is P, X₄₇ is L, X₄₈ is R, X₄₉ is A, X₅₀ is D, X₅₁ is S, X₅₂ is T, X₅₃ is T, X₅₄ is F, X₅₆ is A, X₅₇ is K, X₅₈ is P, X₅₉ is S, X₆₀ is G, X₆₁ is N, X₆₂ is L, X₆₃ is R, X₆₄ is A, X₆₅ is R, X₆₆ is G, X₆₇ is S, X₆₈ is G, X₆₉ is D, X₇₀ is F, X₇₁ is P, X₇₂ is A, X₇₃ is L, X₇₄ is A, X₇₅ is D, X₇₆ is W, X₇₇ is V, X₇₈ is P and X₇₉ is S,

the polypeptide of sequence SEQ ID No172 which corresponds to the sequence SEQ ID No151 in which X₁ is D, X₂ is A, X₃ is Q, X₄ is C, X₅ is V, X₆ is R, X₇ is L, X₉ is D, X₁₀ is G, X₁₁ is G, X₁₂ is S, X₁₃ is S, X₁₅ is H, X₁₆ is A, X₁₇ is E, X₁₈ is A, X₂₀ is P, X₂₁ is L, X₂₃ is L, X₂₄ is P, X₂₅ is M, X₂₆ is G, X₂₇ is M, X₂₉ is V, X₃₀ is G, X₃₁ is Q, X₃₂ is L, X₃₃ is V, X₃₄ is A, X₃₅ is P, X₃₆ is V, X₃₇ is Q, X₃₈ is M, X₄₀ is V, X₄₁ is H, X₄₃ is T, X₄₄ is R, X₄₅ is I, X₄₆ is P, X₄₇ is S, X₄₈ is R, X₄₉ is V, X₅₀ is D, X₅₁ is S, X₅₂ is I, X₅₃ is T, X₅₄ is F, X₅₆ is V, X₅₇ is N, X₅₈ is P, X₅₉ is S, X₆₀ is G, X₆₁ is N, X₆₂ is L, X₆₃ is R, X₆₄ is A, X₆₅ is R, X₆₆ is G, X₆₇ is S, X₆₈ is G, X₆₉ is D, X₇₀ is S, X₇₁ is P, X₇₂ is A, X₇₃ is L, X₇₄ is A, X₇₅ is D, X₇₆ is W, X₇₇ is V, X₇₈ is P and X₇₉ is L,

the polypeptide of sequence SEQ ID No173 which corresponds to the sequence SEQ ID No151 in which X₁ is D, X₂ is A, X₃ is Q, X₄ is Y, X₅ is V, X₆ is H, X₇ is P, X₉ is D, X₁₀ is G, X₁₁ is A, X₁₂ is S, X₁₃ is S, X₁₅ is R, X₁₆ is A, X₁₇ is H, X₁₈ is A, X₂₀ is L, X₂₁ is L, X₂₃ is P, X₂₄ is P, X₂₅ is M, X₂₆ is G, X₂₇ is T, X₂₉ is A, X₃₀ is V, X₃₁ is Q, X₃₂ is P, X₃₃ is A, X₃₄ is V, X₃₅ is P, X₃₆ is A, X₃₇ is Q, X₃₈ is M, X₄₀ is A, X₄₁ is P, X₄₃ is T, X₄₄ is R, X₄₅ is I, X₄₆ is P, X₄₇ is L, X₄₈ is R, X₄₉ is A, X₅₀ is D, X₅₁ is S, X₅₂ is T, X₅₃ is I, X₅₄ is F, X₅₆ is A, X₅₇ is R, X₅₈ is P, X₅₉ is S, X₆₀ is G, X₆₁ is N, X₆₂ is L, X₆₃ is R, X₆₄ is A, X₆₅ is R, X₆₆ is G, X₆₇ is F, X₆₈ is G, X₆₉ is D, X₇₀ is S, X₇₁ is P, X₇₂ is D, X₇₃ is P, X₇₄ is A, X₇₅ is D, X₇₆ is W, X₇₇ is V, X₇₈ is L and X₇₉ is S,

the polypeptide of sequence SEQ ID No174 which corresponds to the sequence SEQ ID No151 in which X₁ is S, X₂ is V, X₃ is Q, X₄ is C, X₅ is A, X₆ is R, X₇ is L, X₉ is V, X₁₀ is G, X₁₁ is A, X₁₂ is N, X₁₃ is S, X₁₅ is R, X₁₆ is A, X₁₇ is E, X₁₈ is A, X₂₀ is L, X₂₁ is P, X₂₃ is L, X₂₄ is P, X₂₅ is M, X₂₆ is G, X₂₇ is M, X₂₉ is A, X₃₀ is G, X₃₁ is Q, X₃₂ is P, X₃₃ is A, X₃₄ is A, X₃₅ is L, X₃₆ is R, X₃₇ is Q, X₃₈ is M, X₄₀ is V, X₄₁ is H, X₄₃ is I, X₄₄ is R, X₄₅ is I, X₄₆ is P, X₄₇ is S, X₄₈ is R, X₄₉ is A, X₅₀ is D, X₅₁ is S, X₅₂ is T, X₅₃ is I, X₅₄ is F, X₅₆ is A, X₅₇ is R, X₅₈ is P, X₅₉ is S, X₆₀ is G, X₆₁ is N, X₆₂ is P, X₆₃ is G, X₆₄ is P, X₆₅ is R, X₆₆ is G, X₆₇ is Y, X₆₈ is G, X₆₉ is G, X₇₀ is S, X₇₁ is R, X₇₂ is A, X₇₃ is H, X₇₄ is A, X₇₅ is D, X₇₆ is W, X₇₇ is V, X₇₈ is P and X₇₉ is S,

the polypeptide of sequence SEQ ID No175 which corresponds to the sequence SEQ ID No151 in which X₁ is G, X₂ is V, X₃ is Q, X₄ is C, X₅ is T, X₆ is R, X₇ is L, X₉ is V, X₁₀ is G, X₁₁ is A, X₁₂ is N, X₁₃ is S, X₁₅ is R, X₁₆ is A, X₁₇ is E, X₁₈ is A, X₂₀ is L, X₂₁ is P, X₂₃ is L, X₂₄ is P, X₂₅ is M, X₂₆ is G, X₂₇ is M, X₂₉ is A, X₃₀ is G, X₃₁ is Q, X₃₂ is P, X₃₃ is A, X₃₄ is A, X₃₅ is L, X₃₆ is R, X₃₇ is Q, X₃₈ is M, X₄₀ is V, X₄₁ is P, X₄₃ is I, X₄₄ is R, X₄₅ is I, X₄₆ is P, X₄₇ is S, X₄₈ is H, X₄₉ is A, X₅₀ is D, X₅₁ is S, X₅₂ is A, X₅₃ is T, X₅₄ is F, X₅₆ is A, X₅₇ is R, X₅₈ is P, X₅₉ is S, X₆₀ is G, X₆₁ is N, X₆₂ is P, X₆₃ is G, X₆₄ is L, X₆₅ is R, X₆₆ is G, X₆₇ is Y, X₆₈ is G, X₆₉ is D, X₇₀ is S, X₇₁ is R, X₇₂ is A, X₇₃ is H, X₇₄ is A, X₇₅ is D, X₇₆ is W, X₇₇ is V, X₇₈ is P and X₇₉ is S, and

the polypeptide of sequence SEQ ID No176 which corresponds to the sequence SEQ ID No151 in which X₁ is S, X₂ is V, X₃ is R, X₄ is C, X₅ is A, X₆ is H, X₇ is L, X₉ is V, X₁₀ is G, X₁₁ is A, X₁₂ is S, X₁₃ is S, X₁₅ is R, X₁₆ is A, X₁₇ is E, X₁₈ is A, X₂₀ is L, X₂₁ is P, X₂₃ is L, X₂₄ is P, X₂₅ is M, X₂₆ is G, X₂₇ is M, X₂₉ is A, X₃₀ is D, X₃₁ is Q, X₃₂ is P, X₃₃ is A, X₃₄ is A, X₃₅ is L, X₃₆ is R, X₃₇ is Q, X₃₈ is M, X₄₀ is V, X₄₁ is P, X₄₃ is I, X₄₄ is R, X₄₅ is T, X₄₆ is P, X₄₇ is S, X₄₈ is H, X₄₉ is A, X₅₀ is D, X₅₁ is S, X₅₂ is T, X₅₃ is T, X₅₄ is F, X₅₆ is A, X₅₇ is R, X₅₈ is P, X₅₉ is S, X₆₀ is G, X₆₁ is N, X₆₂ is P, X₆₃ is G, X₆₄ is P, X₆₅ is R, X₆₆ is G, X₆₇ is Y, X₆₈ is G, X₆₉ is G, X₇₀ is S, X₇₁ is P, X₇₂ is A, X₇₃ is H, X₇₄ is A, X₇₅ is D, X₇₆ is W, X₇₇ is V, X₇₈ is P and X₇₉ is Q.

Preferably, the polypeptide F′ is the polypeptide of sequence SEQ ID No.152.

The applicant has also isolated, unexpectedly, from these polypeptides F′, 4 T epitopes of 9 amino acids, that induce an immune response against the hepatitis C virus.

Thus, another subject of the invention consists of an epitope derived from the protein sequence of the polypeptide F′, characterized in that it induces an immune response against the hepatitis C virus and consists of 9 amino acids located between positions 40 and 48 of the hepatitis C virus polyprotein.

The first epitope isolated, which will subsequently be called A9L, is partially included in the polypeptide F′ in the sense that it begins at position 40. It therefore exhibits a shift of two amino acids relative to the polypeptide F′.

According to a particular embodiment, the A9L epitope has one of the sequences SEQ ID No.177 to SEQ ID No.235, preferably one of the sequences SEQ ID No.177, SEQ ID Nos.183 to 186, SEQ ID Nos.188 to 193, SEQ ID No.201, SEQ ID No.202, SEQ ID No.217, SEQ ID No.218, SEQ ID No.227, SEQ ID No.228 and SEQ ID No.235, the epitope of sequence SEQ ID No.177 being particularly preferred.

According to another subject, the invention relates to the second epitope, subsequently called W9L, characterized in that it induces an immune response specific for the hepatitis C virus and consists of 9 amino acids located between positions 43 and 51 of the hepatitis C virus polyprotein.

According to a particular embodiment, the W9L epitope has one of the sequences SEQ ID No.236 to SEQ ID No.283, preferably one of the sequences SEQ ID No.236, SEQ ID No.241, SEQ ID Nos.248-251, SEQ ID No.253, SEQ ID No.255, SEQ ID No.256 and SEQ ID No.259, the epitope of sequence SEQ ID No.236 being particularly preferred.

According to yet another subject, the invention relates to a third epitope, called R9V, characterized in that it induces an immune response against the hepatitis C virus and consists of 9 amino acids located between positions 50 and 58 of the hepatitis C virus poly-protein.

According to a particular embodiment, the R9V epitope has one of the sequences SEQ ID No.284 to SEQ ID No.358, preferably one of the sequences SEQ ID No.284, SEQ ID No.291, SEQ ID Nos.293 to 295 and SEQ ID Nos.299 to 301, the epitope of SEQ ID No.284 being particularly preferred.

According to yet another subject, the invention relates to a fourth epitope, called G9L, characterized in that it induces an immune response against the hepatitis C virus and consists of 9 amino acids between positions 73 and 81 of the hepatitis C virus polyprotein.

According to a particular embodiment, the G9L epitope has one of the sequences SEQ ID No. 359 to SEQ ID No.434, preferably one of the sequences SEQ ID No. 359, SEQ ID Nos.383 to 386, SEQ ID Nos.388 to 393 and SEQ ID Nos.396 to 400, the epitope of sequence SEQ ID No.359 being particularly preferred.

The present invention also relates to the nucleotide sequences encoding any one of the polypeptides F′ as defined by the sequences SEQ ID Nos. 1 to 176, and also to the nucleotide sequences encoding any one of the epitopes as defined by the sequences SEQ ID Nos.177 to 434.

The polypeptides F′ and epitopes of the invention can be obtained by means of the genetic engineering technique, which comprises the steps consisting in:

culturing a microorganism or eukaryotic cells transformed with a nucleotide sequence according to the invention, and

recovering the peptide produced by said microorganism or said eukaryotic cells.

This technique is well known to those skilled in the art. For further details relating thereto, reference may be made to the following work: Recombinant DNA Technology I, Editors Ales Prokop, Raskesh K Bajpai; Annals of the New York Academy of Sciences, Volume 646, 1991.

The polypeptides F′ and epitopes of the invention can also be prepared by conventional peptide syntheses that are well known to those skilled in the art.

The nucleotide sequences according to the invention can be prepared by chemical synthesis and genetic engineering using the techniques well known to those skilled in the art and described, for example, in J. Sambrook et al., Molecular Cloning: A Laboratory Manual, 1989.

The nucleotide sequences of the invention can be inserted into expression vectors in order to prepare the polypeptides F′ and epitopes of the invention.

Thus, another subject of the invention consists of the expression vectors comprising a nucleotide sequence of the invention, and also the means required for its expression.

By way of an expression vector, mention may be made, for example, of plasmids, viral vectors of the vaccinia virus, adenovirus or baculovirus type, or bacterial vectors of the Salmonella, BCG or listeria type.

The expression “means required for the expression of a polypeptide or epitope” is intended to mean any means that makes it possible to obtain the peptide, such as in particular a promoter, a transcription terminator, an origin of replication and preferably a selection marker.

The vectors of the invention can also comprise sequences required for targeting the peptides to specific cellular compartments. An example of targeting may be the targeting to the endoplasmic reticulum obtained using targeting sequences of the type such as the leader sequence derived from the adenoviral E3 protein (Ciernik I. F., et al., The Journal of Immunology, 199, 162, 3915-3925).

The expression vectors of the invention can comprise either a single nucleotide sequence encoding any one of the polypeptides or epitopes of the invention, or at least two nucleotide sequences encoding different epitopes.

The expression “at least two nucleotide sequences encoding different epitopes” is intended to mean either two nucleotide sequences encoding the following epitope combinations: A9L/W9L, A9L/R9V, A9L/G9L, W9L/R9V, W9L/G9L and R9V/G9L, or three nucleotide sequences encoding the following epitope combinations: A9L/W9L/R9V, A9L/W9L/G9L and W9L/R9V/G9L, or four nucleotide sequences encoding the four epitopes A9L/W9L/R9V/G91, it being understood that the order of the nucleotide sequences is of little importance.

When the expression vectors of the invention comprise several nucleotide sequences, said sequences can be directly linked to one another, or else linked via spacers or linkers that typically consist of small neutral molecules such as amino acids or amino acid mimetics that typically have a neutral charge under physiological conditions.

By way of spacers, mention may be made of Ala or Gly residues or other neutral spacers consisting of non-polar amino acids or of neutral polar amino acids.

These spacer amino acids have at least one or two residues, and conventionally from 3 to 6 residues.

A subject of the invention is also the microorganisms and the eukaryotic cells transformed with an expression vector of the invention.

When it is desired to obtain a composition of the invention containing at least two epitopes of the invention, the microorganisms or eukaryotic cells are transformed with an expression vector containing at least two nucleotide sequences, or else they are co-transformed with at least two expression vectors containing a single nucleotide sequence, each vector encoding an epitope of different type.

By way of examples of microorganisms that are suitable for the purposes of the invention, mention may be made of yeasts such as those of the following families: Saccharomyces, Schizosaccharomyces, Kluveromyces, Pichia, Hanseluna, Yarowia, Schwaniomyces and Zygosaccharomyces, Saccharomyces cerevisiae, Saccharomyces carlsbergensis and Kluveromyces lactis being preferred; and bacteria, such as E. coli and those of the following families: Lactobacillus, Lactococcus, Salmonella, Streptococcus, Bacillus and Streptomyces.

By way of examples of eukaryotic cells, mention may be made of cells originating from animals such as mammals, reptiles, insects and equivalent. The preferred eukaryotic cells are cells originating from the Chinese hamster (CHO cells), from monkey (COS and Vero cells), from baby hamster kidney (BHK cells), from pig kidney (PK 15 cells) and from rabbit kidney (RK13 cells), human osteosarcoma cell lines (143 B cells), HeLa human cells lines and human hepatoma cell lines (of the Hep G2 cell type), and also insect cell lines (for example, Spodoptera frugiperda cell lines).

The host cells can be provided in cultures in suspension or in a flask, or in tissue cultures or organ cultures, and equivalent. The host cells can also be transgenic animals.

The invention also relates to antibodies directed against one of the polypeptides F′ or against one of the epitopes of the invention as defined above.

The antibodies according to the invention are either polyclonal antibodies or monoclonal antibodies.

The abovementioned polyclonal antibodies can be obtained by immunizing an animal with at least one antigen of interest, followed by recovery of the desired antibodies in purified form, by taking a serum sample from said animal and separating said antibodies from the other serum constituents, in particular by affinity chromatography on a column to which is attached an antigen specifically recognized by the antibodies, in particular an antigen of interest.

The monoclonal antibodies can be obtained by the hybridoma technique, the general principle of which is recalled hereinafter.

In a first step, an animal, generally a mouse (or cells in culture in the case of in vitro immunizations), is immunized with an antigen of interest, for which the B lymphocytes are capable of producing antibodies against said antigen. These antibody-producing lymphocytes are then fused with “immortal” myeloma cells (murine in the example) to give hybridomas. The cells capable of producing a particular antibody and of multiplying indefinitely are then selected from the heterogeneous mixture of the cells thus obtained. Each hybridoma is multiplied in the form of a clone, each one resulting in the production of a monoclonal antibody whose properties of recognition with respect to the antigen of interest may be tested, for example by ELISA, by one- or two-dimensional immunoblotting, by immunofluorescence, or using a biosensor. The monoclonal antibodies thus selected are subsequently purified, in particular according to the affinity chromatography technique described above.

The polypeptides F′ and epitopes of the invention are particularly useful for inhibition, prevention and treatment of the virus or of the infection of patients carrying the virus, which belong more particularly to genotypes 1b and 3, such that their use for preparing a drug constitutes another subject of the invention. The present invention also relates to a pharmaceutical composition, in particular a vaccine, containing, as active substance, at least one of the polypeptides F′ as defined above, or else at least epitopes as defined above, or else at least one nucleotide sequence as described above, placed under the control of elements required for constitutive and/or inducible expression of said polypeptides or epitopes, or else at least one antibody as defined above, in combination with a pharmaceutically appropriate vehicle.

The expression “elements required for constitutive expression of the polypeptides or epitopes” is intended to mean a promoter ubiquitous or specific for eukaryotic cells.

By way of elements required for inducible expression of the polypeptides or epitopes, mention may be made of the regulatory elements of the E. coli operon for tetracycline resistance (Gossen M. et al., Proc Natl Acad Sci USA, 89: 5547-5551 (1992)).

Of course, those skilled in the art will readily determine the pharmaceutically appropriate vehicle and the amount of polypeptides, epitopes or antibodies to be used according to the constituents of the pharmaceutical composition.

The amount and the nature of the pharmaceutically appropriate vehicle can be readily determined by those skilled in the art. They are chosen according to the pharmaceutical form and the mode of administration desired.

The pharmaceutical compositions of the invention are suitable for oral, sublingual, subcutaneous, intra-muscular, intravenous, topical, local, intratracheal, intranasal, transdermal, rectal, intraocular or intra-auricular administration, it being possible for said active principle to be administered in a unit form of administration.

The unit forms of administration may, for example, be tablets, gelatin capsules, granules, powders, injectable oral solutions or suspensions, transdermal patches, sublingual, buccal, intratracheal, intraocular, intranasal or intra-auricular administration forms, forms of administration by inhalation, topical, transdermal, subcutaneous, intra-muscular or intravenous administration forms, rectal administration forms, or implants. For topical administration, creams, gels, ointments, lotions or eye lotions can be envisioned.

These pharmaceutical forms are prepared according to the usual methods in the fields under consideration.

Said unit forms contain a dosage so as to allow daily administration of 0.001 to 10 mg of active principle per kg of body weight, according to the pharmaceutical form.

There may be particular cases where higher or lower doses are appropriate; such doses do not depart from the context of the invention. According to the usual practice, the dosage appropriate for each patient is determined by the physician according to the method of administration and the weight and response of the patient.

According to another embodiment of the invention, the present invention also relates to a method of treating pathologies associated with the hepatitis C virus, which comprises the administration, to a patient, of an effective dose of a medicinal product of the invention.

Besides a therapeutic application, the invention also has a diagnostic application in the sense that the polypeptides, the nucleotide sequences encoding said polypeptides and the antibodies of the invention can be used as binding partners in said assays.

The polypeptide and the antibodies of the invention can be used in immunoassays, such as the ELISA assay, and the nucleotide sequences can be used in hybridization assays.

Examples of diagnostic methods comprise, without any implied limitation, blots, “sandwich” techniques, competition techniques and PCR detection techniques, in particular those referred to as “real-time”.

The invention also relates to a diagnostic composition for detecting and/or quantifying the hepatitis C virus, comprising at least one of the polypeptides F′ as defined above, at least one of the nucleotide sequences encoding said polypeptides, as defined above, or else at least one antibody as defined above.

Here again, those skilled in the art will readily determine the amount of polypeptides, nucleotide sequences or antibodies to be used according to the diagnostic technique used.

The invention also relates to a method for detecting and/or quantifying the hepatitis C virus in a biological sample taken from an individual who may be infected with said virus, such as plasma, serum or tissue, characterized in that it comprises the steps consisting in:
bringing said biological sample into contact with the antibodies of the invention under conditions that allow the formation of a complex between the virus and the antibody, and
detecting and/or quantifying the formation of said complex by any appropriate means.

The methods for detecting and/or quantifying the virus are carried out using conventional techniques well known to those skilled in the art, and, by way of illustration, mention may be made of blots, “sandwich” techniques, competition techniques and PCR detection techniques, in particular those referred to as “real-time”.

The invention also relates to the use of the compositions of the invention, for the in vitro diagnosis of the hepatitis C virus in a biological sample or specimen.

Finally, the invention relates to the use of the compositions of the invention, for preparing a vaccine composition.

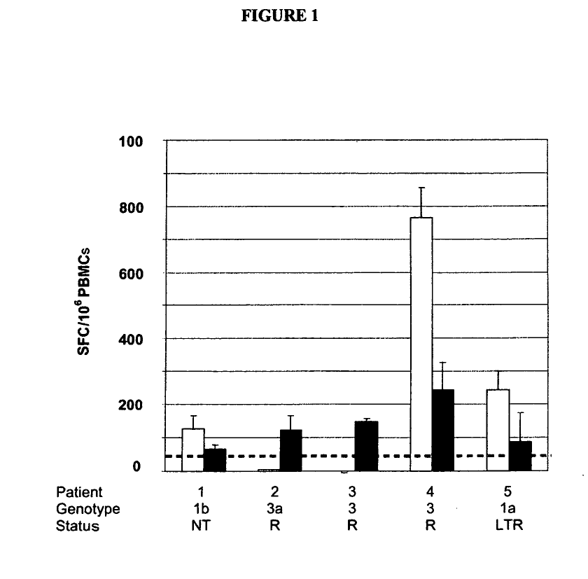

The present invention will be understood more clearly from the following examples given only by way of non-limiting illustration, and also from FIGS. 1, 2 and 3 attached in the appendix, in which:

FIG. 1 represents the production of interferon gamma (IFNγ, white histograms) and of inter-leukin 10 (IL-10, black histograms) by mononuclear cells from 5 HCV-seropositive patients in response to the polypeptide F′ of sequence SEQ ID No.2, this production being demonstrated by ELISpot,

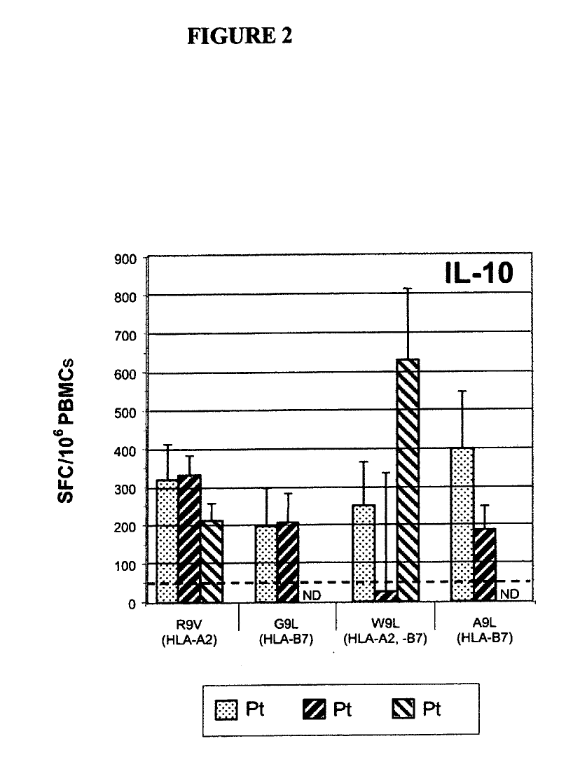

FIG. 2 represents the production of inter-feron gamma (IFNγ, white histograms) and of interleukin (IL-10, black histograms) by mononuclear cells from 3 patients (patient 5 HLA-A2,B18,B35, patient 4 HLA-A2, A24,B27,B62 and patient 6 HLA-A24,A69,B51) in response to the four epitopes A9L (SEQ ID No.177), W9L (SEQ ID No.234), R9V (SEQ ID No.281) AND G9L (SEQ ID No. 355), this production being demonstrated by ELISpot,

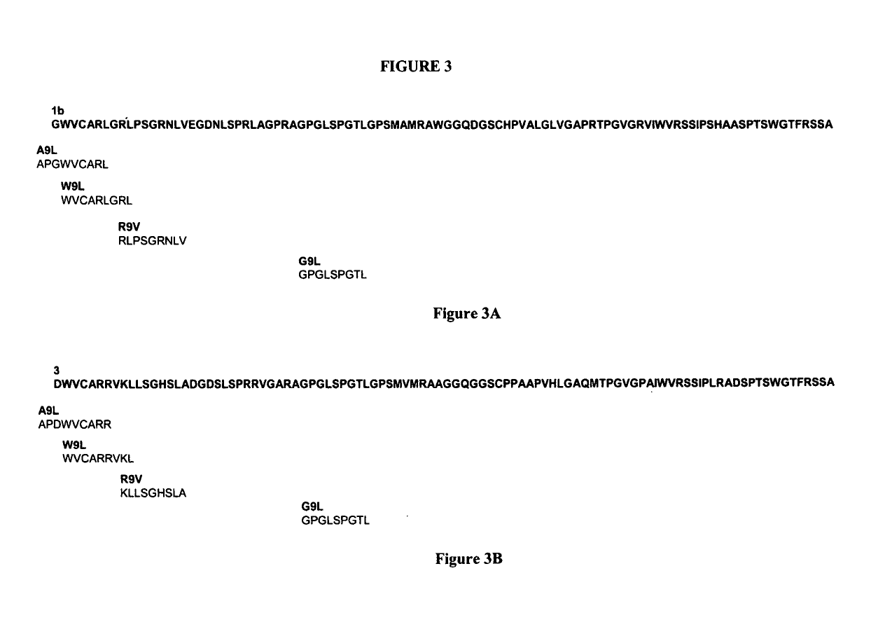

FIG. 3 represents the alignment of the consensus sequences of genotypes 1b (FIG. 3A) and 3 (FIG. 3B), with the A9L, W9L, R9V and G9L epitopes.

### EXAMPLE 1

### Demonstration of an Immune Response against the Polypeptides F′

A sample of approximately 30 ml of blood was taken, on an anticoagulant (EDTA), from 5 HCV-seropositive patients, and the mononuclear cells were purified on a Ficoll gradient.

Two hundred thousand cells thus purified were incubated in RPMI 1640 medium supplemented with 10% of fetal calf serum in the presence or absence of the polypeptide F′ of sequence SEQ ID No.2 at a concentration of 1 μg/ml, for 24 h.

The cells were then transferred into PVDF ELISpot plates which had been preincubated either with an anti-IFNγ antibody or with an anti-IL-10 antibody according to the supplier's recommendations (Diaclone, Besançon, France), and incubated for a further 24 h at 37° C.

After incubation with a biotinylated antibody specific for IFNγ or for IL-10, followed by streptavidin-coupled alkaline phosphatase, the IFNγ-producing or IL-10-producing cells were revealed after degradation of the substrate (NBT/BCIP).

The blue-colored spots corresponding to the cytokine-producing cells were then counted using an automated system (Zeiss microscope, KS-Elispot program).

The results are given in FIG. 1, in a graph representing the number of spot-forming cells (SFC) after deduction of the background noise (medium alone), per 10⁶ mononuclear cells (PBMCs), in the form of histograms (white histograms for the production of IFN gamma and black histograms for the production of IL-10). This graph also gives the status of the patients (NT for nontreated; R, responders; LTR, long-term responder) and also the viral genotype. The horizontal dashed line represents the threshold of significance of the assay and the error bars correspond to the standard deviation between triplicates.

This figure clearly shows that the polypeptides of the invention induce an immune response through the production of IL 10, with or without production of IFN gamma.

### EXAMPLE 2

### Demonstration of an Immune Response with the T Epitopes

In this example, the procedure of Example 1 was repeated, the only difference being that the cells from three patients (Pt 5, Pt 4 and Pt 6) were directly incubated in the presence of the A9L (SEQ ID No.177), W9L (SEQ ID No.234), R9V (SEQ ID No.281) and G9L (SEQ ID No.355) epitopes, in the ELISpot plates for 48 h.

The results are given in FIG. 2, which consists of a graph showing histograms (white for interferon and black for IL10) representing the number of spot-forming cells (SFC) after deduction of the background noise (medium alone), per 10⁶ mononuclear cells (PBMCs). This graph also gives the HLAs to which the prediction relates, i.e. R9V and W9L were predicted to bind to the HLA-A2 molecule and G9L, W9L and A9L were predicted to bind to HLA-B7. The horizontal dashed line represents the threshold of significance of the assay and the error bars correspond to the standard deviation between triplicates. ND means not determined.

Here again, this figure clearly shows that the epitopes of the invention induce an immune response through the production of IL 10, with or without production of IFN gamma.

1. A polypeptide F′ that induces an immune response against the hepatitis C virus, characterized in that it consists of 99 amino acids located between positions 43 and 141 of the hepatitis C virus polyprotein.

2. The polypeptide F′ as claimed in claim 1, characterized in that it has the sequence SEQ ID No.1 below:
X₁WVCX₂X₃X₄X₅RLPSGX₆NX₇X₈X₉X₁₀X₁₁X₁₂LX₁₃X₁₄RX₁₅X₁₆X₁₇PRX₁₈GX₁₉GX₂₀SX₂₁GX₂₂X₂₃GX₂₄SX₂₅X₂₆X₂₇RX₂8X₂₉X₃₀GX₃₁DGSCX₃₂PX₃₃X₃₄X₃₅GLX₃₆GAX₃₇X₃₈TPX₃₉X₄₀GX₄₁X₄₂X₄₃WVX₄₄SSX₄₅X₄₆X₄₇X₄₈X₄₉X₅₀X_{X}₅₁SWGX₅₄X₅₄RX₅₅SX₅₆,
in which
X₁ is G, D, E, V or S, X₂ is A or V, X₃ is R, H or Q, X₄ is L, R, P, S or G, X₅ is G or E, X₆is R, L or H, X₇ is L or P, X₈ is V, E or A, X₉ is E, V, D or G, X₁₀ is G or D, X₁₁ is D or V, X₁₂ is N or S, X₁₃ is S or F, X₁₄ is P or Q, X₁₅ is L, H, R, F, P or C, X₁₆ is A, V or I, X₁₇ is G, S, D, N, I or V, X₁₈ is A, V or E, X₁₉ is P, S or T, X₂₀ is L, P, H or R, X₂₁ is P or L, X₂₂ is T or I, X₂₃ is L, P or H, X₂₄ is P or L, X₂₅ is M or T, X₂₆ is A, V or P, X₂₇ is M, I or T, X₂₈ is A or V, X₂₉ is W, A, L or V, X₃₀ is G or D, X₃₁ is Q, L or R, X₃₂ is H, L, P or R, X₃₃ is V, A, E, K or T, X₃₄ is A or V, X₃₅ is L, R, H or P, X₃₆ is V, A, I or G, X₃₇ is P or L, X₃₈ is R, Q, L, M, T, E or P, X₃₉ is G or D, X₄₀ is V, A or G, X₄₁ is R or H, X₄₂ is V or A, X₄₃ is I or T, X₄₄ is R, G or K, X₄ is I or T, X₄₆ is P or L, X₄₇ is S or L, X₄₈ is H or R, X₄₉ is A or V, X₅₀ is A, V or G, X₅₁ is S or L, X₅₂ is T or I, X₅₃ is T or I, X₅₄ is F, Y or S, X₅₅ is S or L and X₅₆ is V, Gor H.

3. The polypeptide F′ as claimed in claim 2, characterized in that it is chosen from the polypeptides of sequences SEQ ID No.2 to SEQ ID No. 150, preferably the sequence SEQ ID No.2.

4. The polypeptide F′ as claimed in claim 1, characterized in that it has the sequence SEQ ID No. 151 below:
X₁WVCX₂X₃X₄X₅X₅₇LX₅₈X₅₉X₆₀X₆X₆₁X₇AX₉X₁₀X₁₁X₁₂X₆₂X₁₃PX₆₃X₁₅X₁₆X₁₇X₆₄X₆₅X₁₈X₆₆PGX₂₀SX₂₁GTX₂₃GX₂₄X₆₇X₂₅X₂₆X₂₇RAX₂₉X₃₀X₆₈GX₇₀CX₃₂X₇₁X₃₃X₃₄X₃₅X₇₂X₇₃X₃₆GX₇₄X₃₇X₃₈TPGX₄₀X₇₅X₄₁AX₄₃X₇₆X₇₇X₄₄SSX₄₅X₄₆X₄₇X₄₈X₄₉X₅₀X₅₁X₇₈X₅₂SWGX₅₃X₅₄RSX₇₉X₅₆,
in which
X₁ is D, N, S, Y or G, X₂ is A or V, X₃ is R, Q, K or L, X₄is R, Y, C, F, H, L or P, X₅ is V, A or T, X₆is H, R or Q, X₇ is L or P, X₉ is D, V, N, R or T, X₁₀ is G, D or S, X₁₁ is D, V, A, G or E, X₁₂ is S, N or T, X₁₃ is S, P or F, X₁₅ is R, H or L, X₁₆ is V or A, X₁₇ is G, R, E, H or V, X₁₈ is A or D, X₂₀ is L, P or R, X₂₁ is P or L, X₂₃ is L or P, X₂₄ is P or L, X₂₅ is M or T, X₂₆ is V, G, A or E, X₂₇ is M, T or I, X₂₉ is A or V, X₃₀ is G, V or D, X₃₁ is Q or R, X₃₂ is P or L, X₃₃ is A or V, X₃₄ is A or V, X₃₅ is P or L, X₃₆ is L, A, V, R, I or P, X₃₇ is Q, K or P, X₃₈ is M or T, X₄₀ is V, G, D, E or A, X₄₁ is P, H or L, X₄₃ is I or T, X₄₄ is R or K, X₄₅ is I or T, X₄₆ is P or L, X₄₇ is S or L, X₄₈ is R or H, X₄₉ is A or V, X₅₀ is D, G, A or V, X₅₁ is S or L, X₅₂ is T, I or A, X₅₃ is T or I, X₅₄ is F or S, X₅₆ is A or V, X₅₇ is K, R or N, X₅₈ is L, P or Q, X₅₉ is S or N, X₆₀ is G or D, X₆₁ is S or N, X₆₂ is L or P, X₆₃ is R or G, X₆₄ is A, P or L, X₆₅ is R, K, E or T, X₆₆ is G or D, X₆₇ is S, Y or F, X₆₈ is G or W, X₆₉ is G or D, X₇₀ is S or F, X₇₁ is P, H, R or L, X₇₂ is V, A, D or G, X₇₃ is H, L, P, Q or R, X₇₄ is A or P, X₇₅ is G or D, X₇₆ is W or L, X₇₇ is V or A, X₇₈ is P or L and X₇₉ is S, L or Q.

5. The polypeptide F′ as claimed in claim 4, characterized in that it is chosen from the polypeptides of sequence SEQ ID No.152 to SEQ ID No.176, preferably the sequence SEQ ID No.152.

6. A nucleotide sequence encoding any one of the polypeptides F′ as defined in claim 1.

7. An epitope derived from the protein sequence of the polypeptide F′ as defined in claim 1, characterized in that it induces an immune response against the hepatitis C virus and consists of 9 amino acids located between positions 40 and 48 of the hepatitis C virus polyprotein.

8. The epitope as claimed in claim 7, characterized in that it has one of the sequences SEQ ID No.177 to SEQ ID No.235, preferably the sequence SEQ ID No.177.

9. An epitope derived from the protein sequence of the polypeptide F′ as defined in claim 1, characterized in that it induces an immune response against the hepatitis C virus and consists of 9 amino acids located between positions 43 and 51 of the hepatitis C virus polyprotein.

10. The epitope as claimed in claim 9, characterized in that it has one of the sequences SEQ ID No.236 to SEQ ID No.283, preferably the sequence SEQ ID No.236.

11. An epitope derived from the protein sequence of the polypeptide F′ as defined in claim 1, characterized in that it induces an immune response against the hepatitis C virus and consists of 9 amino acids located between positions 50 and 58 of the hepatitis C virus polyprotein.

12. The epitope as claimed in claim 11, characterized in that it has one of the sequences SEQ ID No.284 to SEQ ID No.358, preferably the sequence SEQ ID No.284.

13. An epitope derived from the protein sequence of the polypeptide F′ as defined in claim 1, characterized in that it induces an immune response against the hepatitis C virus and consists of 9 amino acids located between positions 73 and 81 of the hepatitis C virus polyprotein.

14. The epitope as claimed in claim 13, characterized in that it has one of the sequences SEQ ID No.359 to SEQ ID No.434, preferably the sequence SEQ ID No.359.

15. A nucleotide sequence encoding the epitope as defined in claim 7.

16. An expression vector, characterized in that it comprises a nucleotide sequence as claimed in claim 6, and also the means required for its expression.

17. An expression vector, characterized in that it comprises at least two nucleotide sequences as claimed in claim 15, and also the means required for its expression.

18. A microorganism or a host cell transformed with at least one expression vector as defined in claim 17.

19. An antibody directed against the polypeptide F′ as defined in claim 1.

20. (canceled)

21. A pharmaceutical composition, in particular a vaccine, comprising, by way of active substance, at least one of the polypeptides F′ as defined in claim 1, placed under the control of elements required for constitutive and/or inducible expression of said polypeptides F′, in combination with a pharmaceutically appropriate vehicle.

22. A diagnostic composition for detecting and/or quantifying the hepatitis C virus, comprising at least one of the polypeptides F′ as defined in claim 1.

23. A method for detecting and/or quantifying the hepatitis C virus in a biological sample taken from an individual who may be infected with said virus, such as plasma, serum or tissue, characterized in that it comprises the steps consisting in:
bringing said biological sample into contact with the antibodies as claimed in claim 19 under conditions that allow the formation of a complex between the virus and the antibody, and
detecting and/or quantifying the formation of said complex by any appropriate means.

24. (canceled)

25. An expression vector, characterized in that it comprises a nucleotide sequence as claimed in claim 15, and also the means required for its expression.

26. An antibody directed against the epitope as defined in claim 7.

27. An antibody directed against the epitope as defined in claim 9.

28. An antibody directed against the epitope as defined in claim 11.

29. An antibody directed against the epitope as defined in claim 13.

30. A nucleotide sequence encoding the epitope as defined in claim 9.

31. A nucleotide sequence encoding the epitope as defined in claim 11.

32. A nucleotide sequence encoding the epitope as defined in claim 13.
